(19) United States
(12) Patent Application Publication
(10) Pub. No.: US 2013/0274342 A1
(43) Pub. Date: Oct. 17, 2013

(54) COMPOSITIONS AND METHODS FOR TREATING COUGH

(71) Applicant: Cerecor, Inc., Baltimore, MD (US)

(72) Inventors: Mark GINSKI, Perry Hall, MD (US); Blake PATERSON, Baltimore, MD (US); Reza MAZHARI, Townson, MD (US); Djelila MEZAACHE, Stone Ridge, VA (US)

(73) Assignee: Cerecor, Inc., Baltimore, MD (US)

(21) Appl. No.: 13/827,936

(22) Filed: Mar. 14, 2013

Related U.S. Application Data
(60) Provisional application No. 61/623,464, filed on Apr. 12, 2012.
(60) Provisional application No. 61/724,956, filed on Nov. 10, 2012.

(51) Int. Cl. A61K 9/00; A61K 45/06; A61K 31/137; A61K 31/13; A61K 31/09

(52) U.S. Cl. CPC A61K 9/0056; A61K 31/13; A61K 31/09; A61K 31/137; A61K 45/06
USPC 514/655; 514/662

(57) ABSTRACT

Compositions, methods of making compositions and methods of treating cough are described herein. In some embodiments, the compositions are lozenges comprising memantine and an alkalinizing agent.

### CROSS REFERENCE TO RELATED APPLICATIONS

This application claims the benefit of U.S. Provisional Application No. 61/724,956, filed Nov. 10, 2012 and U.S. Provisional Application No. 61/623,464, filed Apr. 12, 2012, which are hereby incorporated by reference in their entirety for all purposes.

### BACKGROUND

Cough is the most common symptom for which patients seek medical advice from primary health care providers. Current antitussive therapies are minimally effective and have side effects that limit their utility. In the United States alone, over 2 billion dollars are spent annually on over the counter cough remedies with questionable efficacy, potential toxicity, and abuse potential, and billions more are spent annually in sick days and doctor's visits. Cough is the primary mechanism of transmission of airborne infections, including all forms of influenza, tuberculosis and Bordetella pertussis, the gram negative bacterium causing whooping cough. As such, cough represents a major public health issue that is poorly treated with currently existing therapies. Currently existing cough medications include dextromethorphan and codeine. People suffering from coughing, sneezing, rhinorrhea, and/or nasal obstruction generally take throat lozenges, cough syrups, and cough drops containing these medications for symptomatic relief. While such medications presently exist, there is room for significant improvement in the composition, efficacy, and adverse effect profiles of these medications.

Other medications currently in the market contain a combination of antitussives, for example one or more expectorants, mucolytics, decongestants, antipyretics, analgesics, or combinations thereof. While such combinations may be acceptable to some patients, others may have restrictions due to allergies or other incompatibilities with certain ingredients. Moreover, the commonly used antitussive agent dextromethorphan has a potential for abuse and because of its lack of potency and side effects profile, has demonstrated limited efficacy in clinical trials.

Furthermore, chronic cough, i.e., a cough lasting longer than eight weeks, is also a common clinical problem for about 11-16% of the population. Ryan et al., Lancet, 2012 August; 380: 1583-89. Although some patients with chronic cough may be treated, the chronic cough can persist after investigation and/or treatment of underlying causes in about 20 to 42% of the patients; these patients are known to have refractory chronic cough. Id. It is now suggested that refractory chronic cough has a neuropathic origin, i.e., the cough is triggered by nontussive origins, such as by neuronal mechanisms or central reflex sensitization. Vertigan et al., Journal of Voice, 2011; 25(5) 596-601. There are thus parallels between refractory chronic cough and chronic pain syndromes. Id. Features of refractory chronic cough include abnormal throat sensation or tickle (laryngeal paraesthesias), increased cough sensitivity in response to known tussigens such as smoke and fumes (hypertussia), and cough triggered by unavoidable non-tussive stimuli such as eating, shortness of breath, talking, physical exercise, and cold air (allotusia). Id. Although current antitussive lozenges may provide some immediate local relief in the throat, they do not also treat the underlying cause of refractory chronic cough. Accordingly, therapies for treating chronic refractory cough are currently needed. Thus, not only is there a need for additional medications that treat/prevent coughing, sneezing, rhinorrhea, and/or nasal obstruction, but for medications that also treat chronic cough associated with chronic pain syndromes. Specifically, new antitussive therapeutics that can provide a dual effect of providing immediate local relief and inhibition of the central neuronal mechanisms associated with refractory chronic cough are particularly desirable.

### SUMMARY OF THE INVENTION

In its various embodiments, the compositions of the present invention comprise a combination of memantine and at least one alkalinizing agent. In a specific embodiment, the composition is an antitussive lozenge. In another embodiment, the lozenge is compressed. In another specific embodiment, the compressed antitussive lozenge comprises memantine, or a pharmaceutically acceptable salt thereof; menthol; and an alkalinizing agent, wherein after a single buccal or sublingual administration, the compressed antitussive lozenge provides a memantine AUC_{0-1 hr} ranging from about 1.0 ng-hr/mL to about 10 ng-hr/mL.

In another embodiment, the compressed antitussive lozenge, after a single buccal or sublingual administration to a patient, provides a memantine AUC_{0-2 hr} ranging from about 5.0 ng-hr/mL to about 15 ng-hr/mL. In another embodiment, after a single buccal or sublingual administration to a patient, the compressed antitussive lozenge provides a memantine AUC_{0-3 hr} ranging from about 12.0 ng-hr/mL to about 20 ng-hr/mL.

In another embodiment, the alkalinizing agent is selected from one or more from the group consisting of aluminum carbonate, aluminum hydroxide, ammonium carbonate, ammonium solution, calcium carbonate, calcium phosphate, diethanolamine, magnesium carbonate, magnesium hydroxide, magnesium oxide, magnesium trisilicate, monoethanolamine, potassium bicarbonate, potassium carbonate, potassium citrate, potassium hydroxide, sodium acetate, sodium bicarbonate, sodium carbonate, sodium citrate, sodium hydroxide, sodium phosphate dibasic, sodium phosphate monobasic, sodium phosphate tribasic, triethanolamine, tromethane and buffering agents sodium carbonate/sodium bicarbonate, barbitone sodium/hydrochloric acid, trisaminomethane/hydrochloric acid, sodium tetraborate/hydrochloric acid, glycine/sodium hydroxide, sodium carbonate/sodium hydrogen carbonate, sodium tetraborate/sodium hydroxide, sodium bicarbonate/sodium hydroxide, sodium hydrogen orthophosphate/sodium hydroxide, and potassium chloride/sodium hydroxide. In another embodiment, the alkalinizing agent is sodium carbonate and sodium bicarbonate.

In another embodiment, the compressed antitussive lozenge has a total weight of about 0.1 g to about 0.5 g. In another embodiment, the total weight of sodium carbonate and sodium bicarbonate in the lozenge is about 1 mg to about 40 mg. In another specific embodiment, the sodium carbonate is present in an amount of about 1 mg to about 12 mg and said sodium bicarbonate is present in an amount of about 5 mg to about 25 mg in the compressed antitussive lozenge. In another embodiment, the sodium carbonate is present in an amount of about 2 mg to about 4 mg and the sodium bicarbonate is present in an amount of about 5 mg to about 10 mg. In another specific embodiment, the sodium carbonate is present in an amount of about 7 mg to about 11 mg and the sodium bicarbonate is present in an amount of about 18 mg to about 24 mg.

In another embodiment of the present invention, the compressed antitussive lozenge includes an amount of memantine of about 1 mg to about 40 mg. In another specific embodiment, the amount of memantine is about 6 mg to about 9 mg.

In another embodiment, after a single buccal or sublingual administration to a patient, the compressed antitussive lozenge provides a memantine Tₘₐₓ ranging from about 10 minutes to about 5.5 hours. In another specific embodiment, after a single buccal or sublingual administration to a patient, the compressed antitussive lozenge provides a memantine Tₘₐₓ ranging from about 10 minutes to about 1.5 hours. In another specific embodiment, after a single buccal or sublingual administration to a patient, the compressed antitussive lozenge provides a memantine Tₘₐₓ ranging from about 2 hours to about 5.5 hours. In another specific embodiment, after a single buccal or sublingual administration to a patient, the compressed antitussive lozenge provides a memantine Cₘₐₓ ranging from about 1 ng/mL to about 2.5 ng/mL per mg dosed. In another specific embodiment, after a single buccal or sublingual administration to a patient, the compressed antitussive lozenge provides a memantine AUC_{0-∞} ranging from about 300 ng-hr/mL to about 1,500 ng-hr/mL.

In another embodiment of the present invention, the compressed antitussive lozenge provides after a single buccal or sublingual administration to a patient a time/plasma concentration curve with two or more peaks (Peak₁ and Peak₂, wherein T₁ refers to the time with the maximum concentration within Peak₁ and T₂ refers to the time with the maximum concentration within Peak₂). In another specific embodiment, the compressed antitussive lozenge provides a memantine T₁ ranging from about 10 minutes to about 1.5 hours after a single buccal or sublingual administration to a patient. In another specific embodiment, the compressed antitussive lozenge provides a memantine T₂ ranging from about 2 hours to about 5.5 hours after a single buccal or sublingual administration to a patient. In another specific embodiment, the compressed antitussive lozenge provides a memantine T₁ ranging from about 10 minutes to about 1.5 hours and a memantine T₂ ranging from about 2 hours to about 5.5 hours after a single buccal or sublingual administration to a patient.

In another embodiment of the present invention, the compressed antitussive lozenge dissolves within about 15 minutes. In another specific embodiment, the compressed antitussive lozenge further comprises one or more excipients selected from the group consisting of a binder, a sugar or sugar substitutes, a filler, a disintegrant, a lubricant, a moisture scavenger and combinations thereof. In another specific embodiment, the excipients comprise microcrystalline cellulose, magnesium stearate, starch, mannitol, sucralose, and magnesium aluminometasilicate.

In another specific embodiment, the compressed antitussive lozenge further includes one or more additional pharmaceutically active ingredients selected from the group consisting of antitussives other than memantine, expectorants, mucolytics, decongestants, nasal decongestants, first generation antihistamines, antihistamines, opioid analgesics, non-opiate analgesics, antipyretics, and combinations thereof. In another embodiment, the one or more additional pharmaceutically active ingredients are selected from the group consisting of guaifenesin, ambroxol, a first generation antihistamine, and combinations thereof.

In various embodiments, the present invention is further directed to methods of treating cough, comprising administering a compressed antitussive lozenge including memantine with an alkalinizing agent to the oral cavity of a patient in need thereof. In another specific embodiment, the methods include administering a compressed antitussive lozenge comprises memantine, or a pharmaceutically acceptable salt thereof; menthol; and an alkalinizing agent to the oral cavity of a patient in need thereof, wherein after a single buccal or sublingual administration, the compressed antitussive lozenge provides a memantine AUC_{0-1 hr} ranging from about 1.0 ng-hr/mL to about 10 ng-hr/mL. In another specific embodiment, the oral administration is buccal administration. In another specific embodiment, the oral administration is sublingual administration.

In another embodiment of the present invention, the methods include administering the compressed antitussive lozenges once a day. In another embodiment of the present invention, the methods include administering the compressed antitussive lozenges at least twice a day.

### BRIEF DESCRIPTION OF THE DRAWINGS

FIG. 1: Effects of pH on memantine solubility and ratio of unionized to ionized form demonstrating a narrow pH window where memantine is substantially unionized and soluble.

FIG. 2: Dose response—Efficacy of oral administration of memantine relative to vehicle control on citric acid-induced cough in guinea pigs; showing dose-dependent antitussive effects of memantine in citric-acid induced guinea pig cough model.

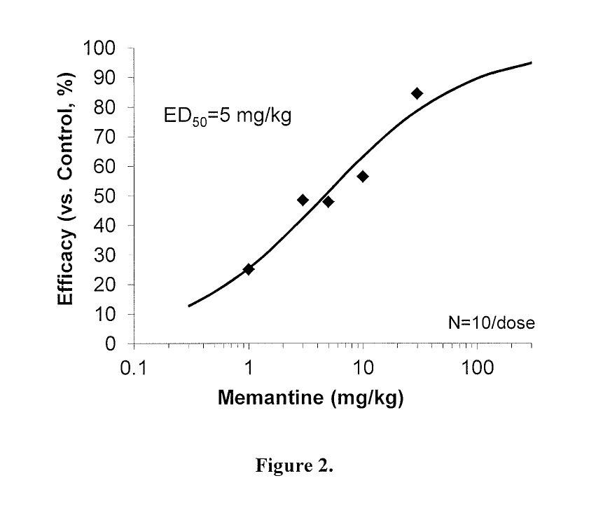

FIG. 3: Memantine-pH dependent in vitro intestinal permeability (based on Caco-2 cells).

FIG. 4: Memantine-pH dependent ex vivo buccal permeability (porcine buccal mucosa).

FIG. 5: Synergistic effect of increasing pH and concurrent use of a permeation enhancer on the rate of permeability of memantine in porcine buccal mucosa (ex vivo).

FIG. 6: Comparison of memantine Caco-2 permeability in the presence of various potential permeation enhancers.

FIG. 7: Representative memantine plasma concentration profile in an individual subject after single dose administration of a compressed lozenge containing memantine, alkalinizing agent and menthol. FIG. 7 shows two peaks (Peak₁ and Peak₂) with a T₁ at about 15 min (C₁ of about 5.6 ng/mL) and a T₂ at about 4 hrs (C₂ at about 7.6 ng/mL).

FIG. 8: Dissolution profile of a compressed lozenge, containing memantine and alkalinizing agent, using a modified USP method (50 rpm paddle speed).

FIG. 9: Mean memantine plasma concentrations in healthy volunteers after administration of 6 mg solution formulation with alkalinizing agents with pH of ˜9.0. FIG. 9 shows an initial peak (Peak₁) with a T₁ at about 15 min (C₁ of about 3 ng/mL) and a T₂ at about 7-8 hrs (C₂ of about 4.3 ng/mL).

### DETAILED DESCRIPTION OF THE INVENTION

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### DEFINITIONS

The term “memantine” as used herein refers to memantine (3,5-dimethyl-1-adamantanamine) as well as any pharmaceutically acceptable salts thereof (e.g., memantine hydrochloride or other salts as described herein), crystalline or amorphous forms (e.g., polymorphs), and solvates (e.g., hydrates, and other crystalline forms in which the crystal structure includes solvent molecules as an integral part of the crystal.

The term “alkalinizing agent” as used herein includes any agent capable of increasing the local pH in the microenvironment of the memantine absorption (e.g., gastrointestinal, oral, sublingual, buccal, gingival or palatal mucosa).

The term “antitussive” broadly refers to agents or compositions which are capable of relieving, suppressing, or reducing the frequency of coughing.

The term “pharmaceutically acceptable” means biologically or pharmacologically compatible for in-vivo use in animals or humans, and can mean approved by a regulatory agency of the Federal or a state government or listed in the U.S. Pharmacopeia or other generally recognized pharmacopeia for use in animals, and more particularly in humans.

The term “Cₘₐₓ” refers to the maximum (or peak) concentration that a drug achieves in the blood plasma after the drug has been administrated and prior to the administration of a second dose.

The term “Tₘₐₓ” refers to the time after dosing at which the maximum or peak concentration of a drug in the blood plasma is achieved after administration of the drug.

The term “AUC” refers to the area under the time/plasma concentration curve after administration of a drug. Total “exposure” of the body of a patient to a drug is often estimated by the AUC_{0-∞}. Partial “exposure” of the body of a patient to a drug is often estimated by the AUC_{0-1 hr}, AUC_{0-2 hr}, AUC_{0-3 hr}, AUC_{0-4 hr}, AUC_{0-5 hr}, AUC_{0-6 hr}, AUC_{0-7 hr}, AUC_{0-8 hr}.

The term “peak” in the time/plasma concentration curve means when the time/plasma concentration curve provides a sharp or gradual increase in the concentration (y axis) over time (x axis), followed by a sharp or gradual decrease in the concentration over time. Accordingly, in a time/plasma concentration curve with two or more peaks, “Peak₁” is the first “peak” in time in the time/plasma concentration curve that provides a sharp or gradual increase in the concentration (y axis) over time (x axis), followed by a sharp or gradual decrease in the concentration over time in the time/plasma concentration curve; “Peak₂” is the second “peak” in time in the time/plasma concentration curve that provides a sharp or gradual increase in the concentration (y axis) over time (x axis), followed by a sharp or gradual decrease in the concentration over time in the time/plasma concentration curve. Accordingly, Peak₁ in the time/plasma concentration curve has a peak drug concentration of “C₁”, which refers to the maximum drug concentration within Peak₁; and a “T₁”, which refers to the time with the maximum concentration within Peak₁, i.e. the time of “C₁”. Accordingly, in a time/plasma concentration curve with at least two peaks, “C₂” in the time/plasma concentration curve refers to the maximum drug concentration within Peak₂; and “T₂” refers to the time with the maximum concentration within Peak₂, i.e. the time of “C₂”. Thus, in a multiple “peak” time/plasma concentration curve, the “Tₘₐₓ” refers to the time (i.e., T₁, T₂, etc.) at which the maximum or highest peak concentration of the multiple peaks occurs. For example, in a time/plasma concentration curve with two peaks, if C₁ is greater than C₂, then T₁ is also the Tₘₐₓ; alternatively, if C₂ is greater than C₁, then T₂ is also the Tₘₐₓ.

The term “t_{1/2}” or “T_{1/2}” refers to the elimination half-life of a drug (i.e., the time required for elimination of half of the peak amount of drug from the body after administration.)

The term “t_{1/2-absorption}” or “T_{1/2-absorption}” refers to the absorption half-life of a drug (i.e., the time required for absorption of half of the peak amount of drug from the body after administration). This is calculated based on the absorption rate, Kₐ, and equals to natural log of 2 divided by Kₐ.

The term “expectorant” refers a compound that works by signaling the body to increase the amount or hydration of secretions, resulting in more yet clearer secretions and as a byproduct lubricating the irritated respiratory tract.

The term “mucolytic” refers to a compound which dissolves thick mucus and is usually used to help relieve respiratory difficulties. It does so by dissolving various chemical bonds within secretions, which in turn can lower the viscosity by altering the mucin-containing components. Both expectorants and mucolytics aid in the clearance of mucous from the airways, lungs, bronchi, and trachea.

The term “antipyretic” refers to compounds which reduced fever. Common antipyretics such as aspirin, non-steroidal anti-inflammatory drugs (NSAID) such as ibuprofen, naproxen, acetaminophen, etc. also have analgesic effects, and may also be referred to as an analgesic/antipyretic or antipyretic/analgesic.

Pharmaceutically acceptable salts include those obtained by reacting the active compound (e.g., memantine), functioning as a base, with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The following are further examples of acid salts that can be obtained by reaction of the active compound (e.g., memantine) with inorganic or organic acids: acetates, adipates, alginates, citrates, aspartates, benzoates, benzenesulfonates, bisulfates, butyrates, camphorates, digluconates, cyclopentanepropionates, dodecylsulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates. For example, the pharmaceutically acceptable salt can be a hydrochloride salt, a hydrobromide salt or a mesylate salt. In one embodiment, the pharmaceutically acceptable salt is a hydrochloride salt.

The term “treating” means one or more of relieving, alleviating, delaying, reducing, reversing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “acute cough” means a condition of sporadic or persistent coughing in a patient for a time period up to about three weeks.

The term “subacute cough” means a condition of sporadic or persistent coughing in a patient for a time period between about three and about eight weeks.

The term “chronic cough” means a condition of sporadic or persistent coughing in a patient for a time period greater than about eight weeks.

An “effective amount” means the amount of a formulation according to the invention that, when administered to a patient for treating a state, disorder or condition is sufficient to effect such treatment. The “effective amount” will vary depending on the active ingredient, the state, disorder, or condition to be treated and its severity, and the age, weight, physical condition and responsiveness of the mammal to be treated.

The term “therapeutically effective” applied to dose or amount refers to that quantity of a compound or pharmaceutical formulation that is sufficient to result in a desired clinical benefit after administration to a patient in need thereof. As used herein with respect to the pharmaceutical formulations comprising memantine, or a pharmaceutically acceptable salt thereof, e.g., memantine hydrochloride, the term “therapeutically effective amount/dose” refers to the amount/dose of the compound that is sufficient to produce an effective response upon administration to a patient.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation. Alternatively, “about” can mean plus or minus a range of up to 20%, up to 10%, or up to 5%.

All weight percentages (i.e., “% by weight” and “wt. %” and w/w) referenced herein, unless otherwise indicated, are measured relative to the total weight of the pharmaceutical composition. As used herein, “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” other active agents would either completely lack other active agents, or so nearly completely lack other active agents that the effect would be the same as if it completely lacked other active agents. In other words, a composition that is “substantially free of” an ingredient or element or another active agent may still contain such an item as long as there is no measurable effect thereof.

The following description includes information that may be useful in understanding the present invention. It is not an admission that any of the information provided herein is prior art or relevant to the presently claimed inventions, or that any publication specifically or implicitly referenced is prior art.

### Memantine Antitussive Effects

The inventors have found that memantine is an extremely effective antitussive. See U.S. patent application Ser. No. 13/272,031, the entire contents of which are herein incorporated by reference for all purposes. As demonstrated in animal models of cough, memantine appears to act centrally by suppressing the cough reflex in the medullary brainstem. Memantine acts in a manner distinct from that of opioids (e.g., codeine), to elevate the threshold for coughing, likely via inhibition of cation flux across the activated NMDA receptor. When compared to the currently approved antitussive dextromethorphan, codeine, and first generation antihistamines, the inventors have found that memantine provides an unexpectedly and very significantly improved antitussive effect, with tolerability and less potential for abuse. In particular, the inventors have found that memantine is significantly and unexpectedly more potent than dextromethorphan, yet does not inhibit NMDA receptors at low levels of glutamate activity, like dextromethorphan (Lipton, Nat Rev Neurosci., 2007 October; 8 (10): 803-8. Review. Erratum in: Nat Rev Neurosci., 2007 November; 8 (11): 2p following 903. Chen et al., J Neurochem., 2006 June; 97 (6): 1611-26).

The inventors have also found that conventional memantine formulations (such as Namenda®) provide insufficient cough relief, due to a relatively long memantine Tₘₐₓ of nearly 8 hours and lack of sufficient exposure in early hours (e.g. insufficient exposure as measured by AUC₀₋₁ and AUC₀₋₂). Further, although conventional memantine compositions have oral bioavailability of greater than 100%, in order to effectively treat cough, it would be desirable to reach maximum plasma concentration of memantine in a much shorter time (i.e., reduce Tₘₐₓ) to provide an immediate reduction in cough frequency, while dose proportionally reducing exposure in order to prevent side effects and maximize safety.

Accordingly, as described herein, the present invention includes compositions comprising memantine that provide, inter alia, higher absorption rates of memantine, and a quicker and more effective cough relief. The present invention thus includes compositions comprising memantine that provide higher absorption rates (Kₐ) and higher AUC₀₋₁ and AUC₀₋₂, which may translate to shorter memantine Tₘₐₓ values (e.g., less than about 3 hours), which is optimal for antitussive therapy.

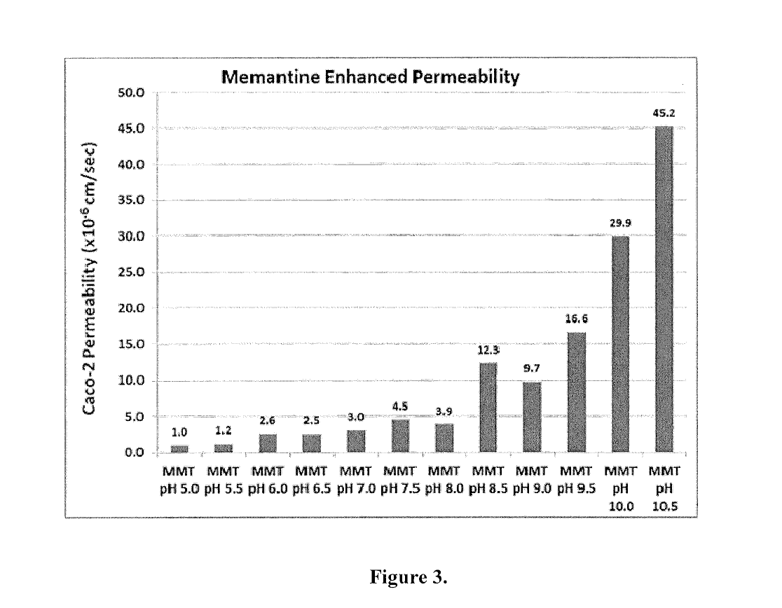

Memantine, however, may be completely ionized at physiological pH, including the physiological pH of the oral cavity and the physiological pH of the GI tract. In other words, memantine may be ionized with at least the pH range of about 1-8. This ionization of memantine thus substantially reduces the ability of memantine to be passively absorbed. According to the Henderson-Hasselbach equation, increasing the pH (alkalization) should reduce the level of ionization of memantine, thus increasing its passive transcellular permeability through the epithelium of the digestive tract, as shown by its permeability through Caco-2 cells (FIG. 3). The permeability of memantine in Caco-2 cells gradually increases with an increase in pH from pH 5.0 to 10.5.

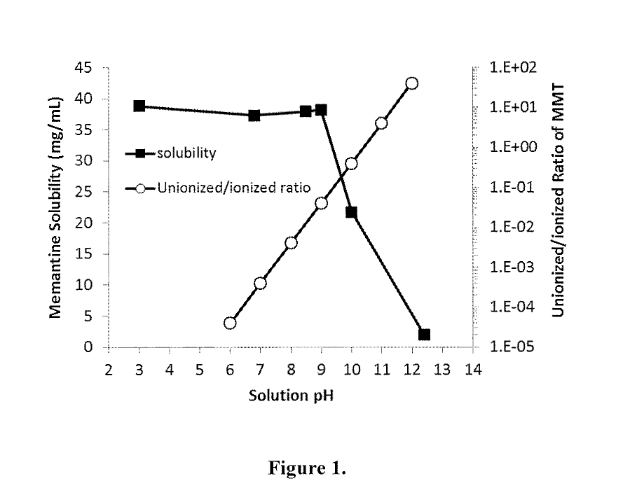

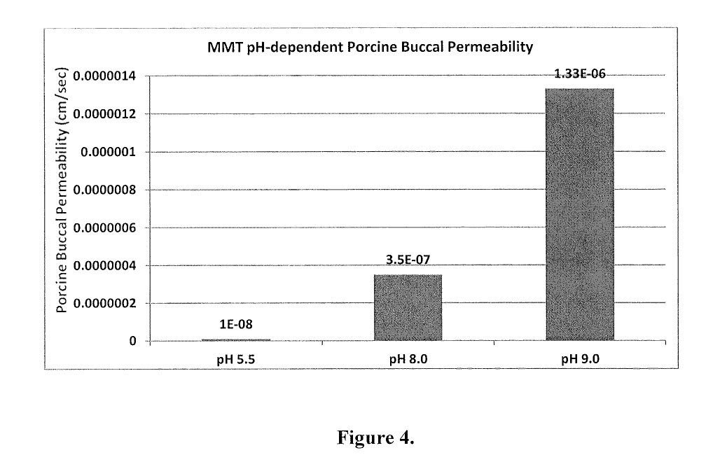

Similar results were found when tested for memantine permeability across ex vivo oral mucosa. It was found that an increase in pH from 5.5 to 9.0 resulted in an over 100-fold increase in permeability of memantine in porcine buccal mucosa (FIG. 4). Thus, compositions of the present invention may include an effective amount of an alkalinizing agent to increase the local pH in the microenvironment at the memantine absorption site, thereby increasing the rate of uptake of memantine. Furthermore, there is a narrow window where memantine is moderately unionized and soluble to achieve concentrations that would facilitate absorption through the oral mucosa (FIG. 1). Use of alkalinizing agents with buffering capacity would thus enable a stable pH range with known unionized to ionized memantine ratio and further increase the rate of memantine absorption.

Accordingly, the compositions of the present invention may include memantine with an alkalinizing agent, and specifically, with an alkalinizing agent with buffering capacity. In a specific embodiment, the compositions of the present invention may be in various dosage forms, such as, for example, a lozenge, a solution, an oral tablet, and an ODT.

In the compositions of the present invention, memantine can be used in the form of the free-base, or in the form of a pharmaceutically acceptable salt. Suitable salts of memantine include, but are not limited to, the acid addition salts disclosed herein. In a particular embodiment, the salt is memantine hydrochloride. All of these salts (or other similar salts) may be prepared by conventional means. All such salts are acceptable provided that they are non-toxic and do not substantially interfere with the desired pharmacological activity.

### Lozenge Formulations

Lozenges are solid pharmaceutical compositions that are intended to dissolve slowly in the mouth, for example over a period of 30-45 minutes. However, the dissolution rate of a particular lozenge can vary. For some individuals, dissolution may occur over a shorter or longer time period.

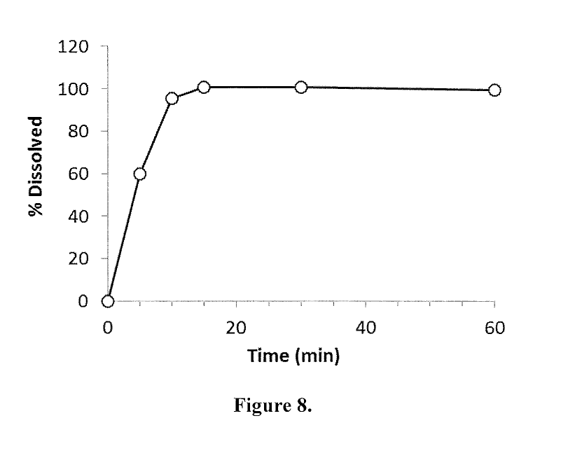

For example, the dissolution of the lozenges may be within a period of about 1, 2, 3, 4, 5, 6, 7, 8, 9, 10, 11, 12, 13, 14, 15, 20, 25, 30, 35, 40, 45, 50, 55, or 60 minutes. In a specific embodiment, the dissolution of the lozenge may occur within about 10 minutes. In a specific embodiment, the dissolution of the lozenge may occur within about 15 minutes. In another embodiment, the dissolution periods listed above may occur using a modified USP method (50 rpm paddle speed). For example, FIG. 8 shows a dissolution profile of a compressed lozenge that completely dissolves within about 15 minutes.

In another embodiment, the dissolution may occur when in the oral cavity of a subject. The dissolution rate of lozenges is distinguished from the dissolution rate or disintegration rate of fast melt or orally disintegrating tablets, which are generally intended to dissolve or disintegrate within a few minutes. Lozenges may contain one or more active ingredients usually in a flavored base which is usually sweetened. Lozenges can be prepared by molding or by compression. Molded lozenges may be referred to as candy lozenges or pastilles, while compressed lozenges may be referred to as troches.

In one embodiment of the present invention, the lozenge may comprise memantine. In some embodiments, the memantine is memantine hydrochloride (3,5-dimethyl-1-adamantanamine hydrochloride).

In a specific embodiment, the lozenge comprising memantine may be used as an antitussive to provide symptomatic treatment of cough, specifically as a lozenge. One advantage of a lozenge as the dosage form for the treatment of cough is that it allows the drug to be absorbed via the oral mucosa.

Buccal or sublingual absorption bypasses first pass metabolism, ensuring that absorption is the primary barrier limiting systemic availability of memantine. The buccal/sublingual cavity also presents a less complex matrix for transiently altering pH, when compared to the gastrointestinal tract. The gastrointestinal tract represents a much more complex matrix in which to increase pH since it exhibits many different inherent physiological mechanisms to quickly alter pH. However, in some embodiments, a substantial portion of the memantine in the lozenge can be ingested, whereby significant absorption of memantine occurs in the gastrointestinal tract.

Memantine, however, is a weak base with a dissociation constant (pKa) of ˜10. Because of its pKa, memantine is essentially ionized in the proximal GI tract, limiting its absorption to the distal small bowel and resulting in a delayed time to maximum plasma concentration (Tₘₐₓ) of ˜4-7 hrs. Similarly, the ionization of memantine also affects the absorption of memantine via the oral mucosa, again increasing the Tₘₐₓ. This long Tₘₐₓ limits the potential clinical utility of memantine in cough, as an antitussive should ideally have a rapid onset of action. Accordingly, the consumer compliant lozenges of the present invention provide a faster absorption of memantine and a decreased Tₘₐₓ for a more rapid and effective treatment of cough than is currently provided. Indeed, the memantine lozenges of the present invention may provide higher absorption rates (Kₐ) and higher AUC₀₋₁ and AUC₀₋₂, which may translate to shorter memantine Tₘₐₓ values (e.g., less than about 3 hours), which is optimal for antitussive therapy.

One embodiment of the present invention is a lozenge formulation that would co-deliver memantine and an alkalinizing agent. The inclusion of an alkalinizing agent to the lozenge allows for an increase in the pH, thus reducing the ionization of memantine in the oral cavity, and thereby improving passive absorption of memantine in the oral mucosa. Accordingly, one aspect of the invention relates to a lozenge comprising memantine and an alkalinizing agent.

An important factor in memantine absorption through the oral mucosa is its relative solubility to ionization state as a function of local pH. There is a narrow window where memantine is both moderately unionized and moderately soluble to achieve concentrations that would facilitate absorption through the oral mucosa (FIG. 1). Use of alkalinizing agents with buffering capacity would enable a predictable and stable pH range with known unionized to ionized memantine ratio.

Non-limiting examples of the alkalinizing agent include aluminum carbonate, aluminum hydroxide, ammonium carbonate, ammonium solution, calcium carbonate, calcium phosphate, diethanolamine, magnesium carbonate, magnesium hydroxide, magnesium oxide, magnesium trisilicate, monoethanolamine, potassium bicarbonate, potassium carbonate, potassium citrate, potassium hydroxide, sodium acetate, sodium bicarbonate, sodium carbonate, sodium citrate, sodium hydroxide, sodium phosphate dibasic, sodium phosphate monobasic, sodium phosphate tribasic, triethanolamine, tromethane, and combinations thereof. In some embodiments, the alkalinizing agent is magnesium oxide, potassium carbonate, sodium phosphate tribasic, sodium carbonate, sodium hydroxide and combinations thereof. In other embodiments, the alkalinizing agent is sodium carbonate and/or sodium hydroxide. In yet other embodiments, the alkalinizing agent is sodium hydroxide. In other embodiments, the alkalinizing agent is sodium carbonate.

In another specific embodiment, the alkalinizing agent may be a buffering agent, such as an alkaline buffering agent. Alkaline buffering agents are mixtures of weak bases and their conjugate acid(s), such as, for example, sodium carbonate/sodium bicarbonate, barbitone sodium/hydrochloric acid, trisaminomethane/hydrochloric acid, sodium tetraborate/hydrochloric acid, glycine/sodium hydroxide, sodium carbonate/sodium hydrogen carbonate, sodium tetraborate/sodium hydroxide, sodium bicarbonate/sodium hydroxide, sodium hydrogen orthophosphate/sodium hydroxide, and potassium chloride/sodium hydroxide. In other embodiments, the alkalinizing agent is one or more of aluminum carbonate, aluminum hydroxide, ammonium carbonate, ammonium solution, calcium carbonate, calcium phosphate, diethanolamine, magnesium carbonate, magnesium hydroxide, magnesium oxide, magnesium trisilicate, monoethanolamine, potassium bicarbonate, potassium carbonate, potassium citrate, potassium hydroxide, sodium acetate, sodium bicarbonate, sodium carbonate, sodium citrate, sodium hydroxide, sodium phosphate dibasic, sodium phosphate monobasic, sodium phosphate tribasic, triethanolamine, tromethane, and combinations thereof. In some embodiments, the alkalinizing agent is magnesium oxide, potassium carbonate, sodium phosphate tribasic, sodium carbonate, sodium hydroxide and combinations thereof. In other embodiments, the alkalinizing agent is sodium carbonate and/or sodium hydroxide. In another specific embodiment, the alkalinizing agent is sodium carbonate. In yet other embodiments, the alkalinizing agent is sodium hydroxide. As stated above, the inclusion of an alkalinizing agent such as an alkaline buffering agent to the lozenge allows for an increase in the pH, thus reducing the ionization of memantine in the oral cavity, and thereby improving passive absorption of memantine in the oral mucosa. In addition, buffering agents resist pH changes. Accordingly, adding an alkaline buffering agent may provide more control over the pH in the oral cavity, and thus provide more consistent absorption and consistent pharmacokinetics. In another specific embodiment, the alkalinizing agent is an alkaline buffering agent such as sodium carbonate and sodium bicarbonate.

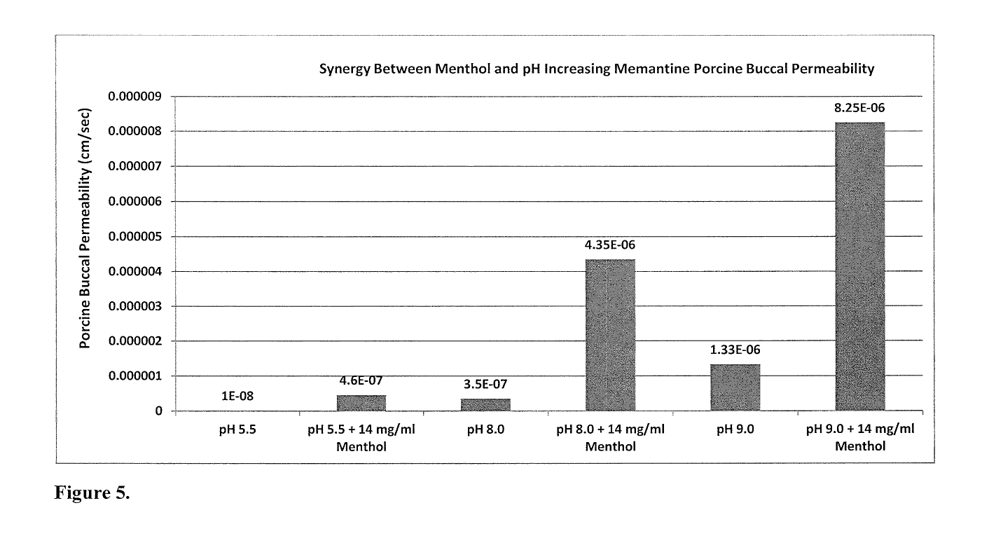

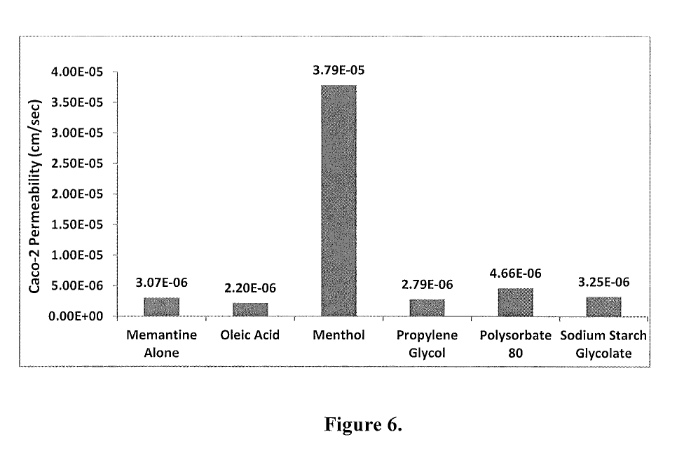

As disclosed herein, an increase in pH was found to increase the rate of passive permeability of memantine in porcine buccal mucosal tissue. Surprisingly and unexpectedly, it was found that the addition of pharmaceutically acceptable excipients, such as a permeation enhancer in combination with an alkalinizing agent or buffer resulted in a significant and synergistic increase in the rate of permeability of memantine (FIG. 5). FIG. 5 shows that an increase in pH from 8.0 to 9.0 resulted in about 3.8 fold increase in permeability. Surprisingly, addition of menthol (14 mg/mL) resulted in about a 6 fold increase at pH 8.0 and about a 12 fold increase at pH 9.0. Such an increased rate of permeability substantially increases the rate of absorption of memantine. Accordingly, in some embodiments, the lozenge further comprises, in addition to the memantine and the alkalinizing agent, one or more permeation enhancers. Various permeation enhancers have been proposed to increase the permeability of drugs through the oral mucosa, such as those disclosed in U.S. Pat. No. 7,682,628 for use with zolpidem compositions. However, the inventors have found that some permeation enhancers which are effective for zolpidem or other drugs are not effective for memantine, and thus appear to have drug-specific activity for enhancing drug permeation. See, for example FIG. 6, which shows that oleic acid, propylene glycol, polysorbate 80, and sodium starch glycolate are ineffective as permeation enhancers, while menthol unexpectedly provides an approximately 10-fold increase in the permeability of memantine.

In some embodiments, the one or more pharmaceutically acceptable excipients comprise one or more permeation enhancers. Non-limiting examples of permeation enhancers include menthol chitosan, resorcinol, surfactants, polyethylene glycol, bioacids (e.g., citric acid, lactic acid), liposomes, polysaccharides, peptide transport agents (e.g., as disclosed in U.S. Pat. No. 7,176,185), dimethylsulfoxide (“DMSO”), dimethyl formamide (“DMF”), N,N-dimethylacetamide (“DMA”), decylmethylsulfoxide (“CIOMSO”), polyethylene glycol monolaurate (“PEGMLIt), glycerol monolaurate, lecithin, 1-substituted azacycloheptan-2-ones (e.g., 1-n-dodecylcyclazacycloheptan-2-one, available as Azone®), lower alkanols (e.g., ethanol), SEPA®, cholic acid, taurocholic acid, bile salt type enhancers, and surfactants (e.g., Tergitol®, Nonoxynol-9®, TWEEN-80®).

In certain embodiments, the one or more permeation enhancers comprise menthol. The menthol may be any stereoisomer (e.g., 1R-, 2S-, 5R-menthol) or combination of stereoisomers. In another embodiment, menthol may be obtained naturally from diverse mint oils or prepared synthetically. Menthol may be levorotatory (l-Menthol), from natural or synthetic sources, or racemic (dl-Menthol) produced synthetically. It may occur as hexagonal crystals, needle like or in fused masses, or as a crystalline powder. The descriptions above for menthol are non-limiting and are exemplary and not intended as limitation on the scope of the invention.

In some embodiments, the lozenge further comprises one or more pharmaceutically acceptable excipients. Non-limiting examples of excipients include sweetening agents, colorants, flavorants, permeation enhancers, solvents, co-solvents, fillers, binders, disintegrants, super-disintegrants, lubricants, glidants, moisture scavengers, diluents, urinary acidification agents, coating agents, ion exchange resins, absorbents, direct compression excipients, opacifiers, polishing agents, suspending agents, anti-adherents, preservatives, clarifying agents, emulsifying agents, antifoaming agents, antioxidants, buffering agents, plasticizers, surfactants, tonicity agents and viscosity increasing agents. In certain embodiments, the lozenge further comprises one or more pharmaceutically acceptable excipients independently selected from sweetening agents, colorants, flavorants, permeation enhancers, solvents, co-solvents, fillers, binders, disintegrants, super-disintegrants, lubricants, glidants, moisture scavengers, diluents, coating agents and ion exchange resins.

In some embodiments, the one or more pharmaceutically acceptable excipients comprise one or more sweetening agents. Non-limiting examples of sweetening agents include sugar, monosaccharides, oligosaccharides, aldose, ketose, dextrose, maltose, lactose, glucose, fructose, sucrose, mannitol, xylitol, sorbitol (e.g., D-sorbitol, L-sorbitol), isomalt, erythritol, pentitol, hexitol, malitol, acesulfame potassium, talin, glycyrrhizin, sucralose, aspartame, saccharin, sodium saccharin, maltodextrin, neohesperidin dihydrochalcone, monoammonium glycyrrhizinate, sodium cyclamate, and combinations thereof. In certain embodiments, the one or more sweetening agents are independently selected from sucralose, isomalt and acesulfame potassium. In other embodiments, the one or more sweetening agents comprise isomalt and acesulfame potassium.

In some embodiments, the one or more pharmaceutically acceptable excipients comprise one or more colorants. Non-limiting examples of colorants include FD&C Blue 1, FD&C Blue 2, FD&C Green 3, FD&C Red 3, FD&C Red 40, FD&C Yellow 5, FD&C Yellow 6, Orange B and Citrus Red 2. In certain embodiments, the one or more colorants comprise FD&C Blue 2 and FD&C Red 40.

In some embodiments, the one or more pharmaceutically acceptable excipients comprise one or more flavorants. Non-limiting examples of flavorants include natural, artificial and synthetic flavor oils, oleoresins, aldehydes, esters, honey, artificial honey flavor, citric acid, malic acid, vanilla, vanillin, cocoa, chocolate, menthol, fruit essences and extracts derived from plants, animals, leaves, flowers, fruits and combinations thereof. Examples of flavor oils include, without limitation, anise oil, cinnamon oil, peppermint oil, spearmint oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil and grape oil. Examples of fruit essences include, without limitation, apple, pear, peach, berry, wildberry, date, blueberry, kiwi, strawberry, raspberry, cherry, black cherry, plum, pineapple and apricot essences. Examples of aldehydes include, without limitation, acetaldehyde (apple); benzaldehyde (cherry, almond); cinnamic aldehyde (cinnamon); citral, i.e., α-citral (lemon, lime); neral, i.e., β-citral (lemon, lime); decanal (orange, lemon); ethyl vanillin (vanilla, cream); heliotropine, i.e., piperonal (vanilla, cream); vanillin (vanilla, cream); α-amyl cinnamaldehyde (spicy fruity flavors); butyraldehyde (butter, cheese); valeraldehyde (butter, cheese); citronellal (modifies, many types); decanal (citrus fruits); aldehyde C-8 (citrus fruits); aldehyde C-9 (citrus fruits); aldehyde C-12 (citrus fruits); 2-ethyl butyraldehyde (berry fruits); hexenal, i.e., trans-2 (berry fruits); tolyl aldehyde (cherry, black cherry, almond); veratraldehyde (vanilla); 2,6-dimethyl-5-heptenal, i.e., melonal (melon); 2-6-dimethyloctanal (green fruit); and 2-dodecenal (citrus, mandarin). In certain embodiments, the one or more flavorants are independently selected from menthol, honey lemon flavor, cherry flavor and black cherry flavor. In other embodiments, the one or more flavorants comprise menthol and black cherry flavor. In further embodiments, the black cherry flavor is selected from FALU906 or FALT098, or a combination thereof.

In some embodiments, the one or more pharmaceutically acceptable excipients comprise one or more binders. Non-limiting examples of binders include starch, gelatin, sugars (e.g., sucrose, glucose, dextrose, molasses, lactose), natural and synthetic gums (e.g., acacia, sodium alginate, extract of Irish moss, panwar gum, ghatti gum, mucilage of isapol husks, carbomethoxycellulose, methylcellulose, polyvinylpyrrolidone, Veegum, larch arabogalactan), polyethylene glycol, ethylcellulose, waxes, water, alcohol and polymers (e.g., hydroxypropyl cellulose, povidone, methylcellulose, hydroxypropyl methylcellulose, carboxyalkyl celluloses, polyethylene oxides, polysaccharides, acacia, alginic acid, agar, calcium carrageenan, sodium carboxymethyl cellulose, microcrystalline cellulose, dextrin, ethylcellulose, gelatin, liquid glucose, guar gum, hydroxypropyl methylcellulose, methylcellulose, pectin, PEG, povidone, pregelatinized starch). In certain embodiments, the one or more binders are independently selected from polyethylene glycol and povidone. In other embodiments, the one or more binders comprise povidone.

In some embodiments, the one or more pharmaceutically acceptable excipients comprise one or more disintegrants. Non-limiting examples of disintegrants include dibasic calcium phosphate, dibasic calcium phosphate dihydrate, tribasic calcium phosphate, alginic acid, hydroxypropyl cellulose, carboxymethyl cellulose calcium, carboxymethyl cellulose sodium, cross-linked carboxymethyl cellulose sodium, swellable ion exchange resins, alginates, formaldehyde-casein, cellulose, croscarmellose sodium, crosspovidone (e.g., cross-linked polyvinyl pyrrolidone), microcrystalline cellulose, sodium carboxymethyl starch, sodium starch glycolate and starches (e.g., corn starch, rice starch). In certain embodiments, the one or more disintegrants comprise microcrystalline cellulose. In another embodiment, the one or more disintegrants may be Pearlitol® Flash. Pearlitol® Flash comprises co-processed mannitol and starch and thus may be a disintegrant and/or a binder.

In some embodiments, the one or more pharmaceutically acceptable excipients comprise one or more lubricants. Non-limiting examples of lubricants include calcium stearate, magnesium stearate, sodium stearyl fumarate, stearic acid, zinc stearate, talc, waxes, Sterotex® Stearowet®, and mixtures thereof. In certain embodiments, the one or more lubricants comprise sodium stearyl fumarate. In other embodiments, the one or more lubricants comprise magnesium stearate.

In some embodiments, the one or more pharmaceutically acceptable excipients comprise one or more diluents. Non-limiting examples of diluents include deionized water, mannitol, sucrose, anhydrous dibasic calcium phosphate, anhydrous dibasic calcium phosphate dihydrate, tribasic calcium phosphate, cellulose, lactose, magnesium carbonate and microcrystalline cellulose. In certain embodiments, the one or more diluents comprise deionized water.

In some embodiments, the one or more pharmaceutically acceptable excipients comprise one or more glidants. Non-limiting examples of glidants include colloidal silicon dioxide and talc.

In some embodiments, the one or more pharmaceutically acceptable excipients comprise one or more buffering agents. The one or more buffering agents can be used to effect pH change in the microenvironment of the absorption site in order to increase the concentration of non-ionized memantine. For example, basic buffering agents such as alkali carbonates can be used to rapidly elevate the pH of a microenvironment. It is also possible to use a binary or ternary buffer system to maintain the pH above 8.5. In some embodiments, the lozenge comprises a buffer system similar to or the same as that disclosed in U.S. Pat. No. 7,658,945, which produces and maintains a final pH above about 8.5. In some embodiments, the lozenge comprises a buffer system which produces a final pH above about 9.0. In another embodiment, the lozenge comprises a buffer system which produces a final pH above about 9.5. In another embodiment, the lozenge comprises a buffer system which produces a final pH above about 10.0. In another embodiment, the lozenge comprises a buffer system which produces a final pH above about 10.5. In another embodiment, the lozenge comprises a buffer system which produces a final pH above about 11.0.

In some embodiments, the one or more pharmaceutically acceptable excipients comprise one or more moisture scavengers. Non-limiting examples of moisture scavengers include calcium silicate, sodium aluminosilicate, sodium metabisulfite and magnesium aluminometasilicate (such as Neusilin®, and specifically, Neusilin® US2). In certain embodiments, the one or more moisture scavengers comprise magnesium aluminometasilicate. In another specific embodiment, the one or more moisture scavenger may comprise sodium metabisulfite.

In some embodiments, the one or more pharmaceutically acceptable excipients comprise one or more fillers. Non-limiting examples of fillers include lactose (e.g. spray-dried lactose, α-lactose, β-lactose, Tabletose®, various grades of Pharmatose®, Microtose® or Fast-Flo®), microcrystalline cellulose (various grades of Avicel®, Ceolus®, Elcema®, Vivacel®, Ming Tai® or Solka-Floc®), hydroxypropylcellulose, L-hydroxypropylcellulose (low substituted), low molecular weight hydroxypropyl methylcellulose (HPMC) (e.g. Methocel E, F and K from Dow Chemical, Metolose SH from Shin-Etsu, Ltd), hydroxyethyl cellulose, sodium carboxymethyl cellulose, carboxymethylhydroxyethyl cellulose and other cellulose derivatives, glucose, fructose, sucrose, agarose, mannose, dextrose, galactose, mannitol, sorbitol, xylitol, dextrins, maltodextrins, starches and modified starches (e.g., potato starch, maize starch, rice starch), co-processed mannitol and starch such as Pearlitol® Flash, calcium phosphate (e.g. basic calcium phosphate, calcium hydrogen phosphate, dicalcium phosphate hydrate), calcium sulfate, calcium carbonate, sodium alginate, collagen, silicon dioxide, titanium dioxide, talc, alumina, starch, kaolin, polacrilin potassium. The one or more fillers may be water insoluble, water soluble or a combination of water insoluble and water soluble fillers. Examples of water insoluble fillers include, without limitation, silicon dioxide, titanium dioxide, talc, alumina, starch, kaolin, polacrilin potassium, powdered cellulose, microcrystalline cellulose, and combinations comprising one or more of the foregoing fillers. Examples of water soluble fillers include, without limitation, sugars (e.g., lactose, glucose, fructose, sucrose, mannose, dextrose and galactose) and sugar alcohols (e.g., mannitol, sorbitol, xylitol). In certain embodiments, the one or more fillers are a combination of water insoluble and water soluble fillers. In other embodiments, the one or more fillers are independently selected from microcrystalline cellulose and sorbitol. In some embodiments, the one or more fillers comprise microcrystalline cellulose. In yet other embodiments, the one or more fillers comprise sorbitol.

In some embodiments, the one or more pharmaceutically acceptable excipients comprise one or more coating agents. The one or more coating agents can help mask the taste of the other components, protect components from atmospheric degradation, improve appearance, retard disintegration, control release of the active ingredient and/or physically separate components (e.g., memantine and alkalinizing agent) to reduce physical or chemical degradation of one or more components (e.g., memantine). For example, the one or more coating agents may protect the memantine from the alkalinizing agent but still permit rapid though slightly delayed released compared to lozenges lacking a coating agent. In certain embodiments, the one or more coating agents encapsulate the memantine, the alkalinizing agent, or both the memantine and the alkalinizing agent. Non-limiting examples of coating agents include silicone elastomers, wax, fatty acids, polymethacrylate copolymers, polyacrylates, shellac, methyl cellulose, hydroxypropyl cellulose, hydroxypropyl methyl cellulose, ethyl cellulose, hydroxyethyl cellulose, hydroxypropyl cellulose, carboxymethyl cellulose, cellulose acetate phthalate, cellulose acetate butyrate, amylose, dextran, casein, pullulan, gelatin, pectin, agar, carrageenan, xanthan gum, tragacanth, guar gum, acacia gum, arabic gum, polyethylene glycol, polyethylene oxide, polyvinyl pyrrolidone (PVP), polyvinyl alcohol, cyclodextrin, carboxyvinyl polymers, sodium alginate, polyacrylic acid, methylmethacrylate, acrylic ester copolymers (e.g., Eudragit NE30D) and amine-functional acrylates (e.g., Eudragit E100, EPO). In certain embodiments, the one or more coating agents comprise a water soluble polymer, a combination of two or more water soluble polymers or a combination of a water soluble polymer and a water insoluble or poorly soluble polymer. In further embodiments, the one or more coating agents are selected from ethyl cellulose and hydroxypropyl cellulose. In other embodiments, the one or more coating agents comprise ethyl cellulose. In yet other embodiments, the one or more coating agents comprise hydroxypropyl cellulose.

In some embodiments, the one or more pharmaceutically acceptable excipients comprise one or more plasticizers. Non-limiting examples of plasticizers include polyethylene glycol, propylene glycol, glycerin, glycerol, monoacetin, diacetin, triacetin, dimethyl phthalate, diethyl phthalate, dibutyl phthalate, dibutyl sebacate, triethyl titrate, tributyl citrate, triethyl citrate, triethyl acetyl citrate, castor oil, acetylated monoglycerides, sorbitol or combinations thereof. In certain embodiments, the one or more plasticizers are selected from polyethylene glycol and propylene glycol. In other embodiments, the one or more plasticizers comprise polyethylene glycol. In yet other embodiments, the one or more plasticizers comprise propylene glycol.

In some embodiments, the one or more pharmaceutically acceptable excipients comprise one or more surfactants. Non-limiting examples of surfactants include sodium docusate, polyoxyethylene ether, poloxamer, polysorbates (Tween), polyoxyethylene stearates, sodium lauryl sulfate and sorbitan esters. In certain embodiments, the one or more surfactants are included in the coating. In other embodiments, the one or more surfactants are used as compressibility augmenting agents.

In some embodiments, the one or more pharmaceutically acceptable excipients comprise one or more ion exchange resins. Non-limiting examples of ion exchange resins include “Dowex” resins and others made by Dow Chemical; “Amberlyte”, “Amberlyst” and other resins made by Rohm and Haas; “Indion” resins made by Ion Exchange, Ltd. (India), “Diaion” resins by Mitsubishi; BioRex Type AG and other resins by BioRad; “Sephadex” and “Sepharose” made by Amersham; resins by Lewatit, sold by Fluka; “Toyopearl” resins by Toyo Soda; “IONAC” and “Whatman” resins, sold by VWR; and “BakerBond” resins sold by J T Baker. In certain embodiments, the one or more ion exchange resins comprise sulfonated polymers, such as polystyrene cross-linked with divinylbenzene. In other embodiments, the one or more ion exchange resins are selected from Amberlite IRP-69, Indion 224, Indion 244 and Indion 254. In further embodiments, the one or more ion exchange resins are complexed with the memantine. The one or more ion exchange resins may protect the memantine from the alkalinizing agent but still permit rapid though slightly delayed release compared to non-complexed memantine.

In some embodiments, the one or more pharmaceutically acceptable excipients are selected to limit or avoid the formation of memantine adducts. Adducts, also called addition compounds, result from the direct combination of two or more different compounds. For example, memantine adduct formation may occur with formulations containing lactose or other reducing sugars. Such adduct formation detracts from the efficacy of the product and increases the risks of other side effects (e.g., lactose-memantine adduct has an antibiotic activity).

In some embodiments, the lozenge further comprises one or more additional pharmaceutically active agents, such as antitussives other than memantine, expectorants, decongestants, nasal decongestants, antihistamines, antipyretics, analgesics, opioids and mucolytics.

In some embodiments, the one or more additional active agents comprise one or more antitussives. Non-limiting examples of antitussives include guaifenesin, dextromethorphan, dextromethorphan hydrobromide, codeine, codeine phosphate, codeine sulfate, hydrocodone, morphine, morphine sulfate, hydromorphone hydrochloride, levorphanol tartrate, fentanyl, fentanyl citrate, oxycodone hydrochloride, oxymorphone hydrochloride, methadone hydrochloride, apomorphine hydrochloride, beechwood creosote, benzonatate, camphor ethanedisulfonate, diphenhydramine, diphenhydramine hydrochloride, chlophendianol hydrochloride, carbetapentane citrate, caramiphen edisylate, noscapine, noscapine hydrochloride and menthol. In certain embodiments, the one or more antitussives comprise guaifenesin.

In some embodiments, the one or more additional active agents comprise one or more decongestants. Non-limiting examples of decongestants include phenylephrine, ephedrine, ephedrine sulfate, ephedrine hydrochloride, pseudoephedrine hydrochloride, phenylephrine hydrochloride, epinephrine bitartrate, hydroxyamphetamine hydrobromide, propylhexedrine, phenylpropanolamine hydrochloride, mephentermine sulfate, methoxamine hydrochloride, naphazoline hydrochloride, oxymetalozine hydrochloride, tetrahydrozoline hydrochloride and xylometazoline hydrochloride. In certain embodiments, the one or more decongestants comprise phenylephrine.

In some embodiments, the one or more additional active agents comprise one or more opioids. Non-limiting examples of opioids include codeine, morphine, hydromorphone, hydrocodone, oxymorphone, levorphanol, fentanyl, propoxyphene, diphenoxylate, meperidine, methadone, oxycodone, butorphanol and morphine.

In some embodiments, the one or more additional active agents comprise one or more expectorants. Non-limiting examples of expectorants include ammonium chloride, ammonium carbonate, acetylcysteine, antimony potassium tartrate, glycerin, potassium iodide, sodium citrate, terpin hydrate and tolu balsam.

In some embodiments, the one or more additional active agents comprise one or more mucolytics. Non-limiting examples of mucolytics include acetylcysteine, ambroxol, bromhexine, carbocisteine, domiodol, dornase alfa, eprazinone, erdosteine, letosteine, mesna, neltenexine, sobrerol, stepronin and tiopronin.

In some embodiments, the one or more additional active agents are selected from guaifenesin and phenylephrine. In certain embodiments, the one or more additional active agents comprise guaifenesin. In other embodiments, the one or more additional active agents comprise phenylephrine. In yet other embodiments, the lozenge is substantially free of active ingredients other than memantine and guaifenesin and/or phenylephrine.

In some embodiments, the lozenge is substantially free of pharmaceutically active agents other than memantine.

In some embodiments, the lozenge is about 0.1 g to about 2 g in weight. In one embodiment, the lozenge is about 0.2 g to about 1.0 g in weight. In another embodiment, the lozenge is about 0.25 g in weight. In some embodiments, the lozenge is about 0.5 g to about 5 g in weight. In one embodiment, the lozenge is about 0.5 g to about 4.5 g in weight. In another embodiment the lozenge is about 1.5 g to about 4.5 g in weight. In another embodiment, the lozenge is about 2 g to about 4 g in weight. In another embodiment, the lozenge is about 2.5 g to about 3.5 g in weight. In certain embodiments, the lozenge weighs about 0.5 g. In other embodiments, the lozenge weighs about 1 g. In other embodiments, the lozenge weighs about 1.5 g. In other embodiments, the lozenge weighs about 2 g. In other embodiments, the lozenge weighs about 2.5 g. In other embodiments, the lozenge weighs about 3 g. In other embodiments, the lozenge weighs about 3.5 g. In other embodiments, the lozenge weighs about 3.5 g. In other embodiments, the lozenge weighs about 4 g. In other embodiments, the lozenge weighs about 4.5 g. In yet other embodiments, the lozenge weighs about 4.75 g. In further embodiments, the lozenge weighs about 5 g.

In some embodiments, the lozenge has a pH of about 7.5 or higher. In some embodiments, the lozenge has a pH of about 8.0, or higher. In some embodiments, the lozenge has a pH of about 8.5, or higher. In some embodiments, the lozenge has a pH of about 9, or higher. In certain embodiments, the lozenge has a pH of about 7.5 to about 11. In particular embodiments, the lozenge has a pH of about 8 to about 11. In certain embodiments, the lozenge has a pH of about 8.5 to about 11. In particular embodiments, the lozenge has a pH of about 9 to about 11. In particular embodiments, the lozenge has a pH of about 9 to about 11. In other embodiments, the lozenge has a pH of about 9 to about 10. In further embodiments, the lozenge has a pH of about 10 to about 11. In other embodiments, the lozenge has a pH of about 7.5. In other embodiments, the lozenge has a pH of about 8. In other embodiments, the lozenge has a pH of about 8.5. In other embodiments, the lozenge has a pH of about 9. In yet other embodiments, the lozenge has a pH of about 10. In additional embodiments, the lozenge has a pH of about 11.

In some embodiments, the lozenge has a moisture content of about 6.0% w/w or lower. In other embodiments, the lozenge has a moisture content of about 0.5 to about 5.5% w/w. In other embodiments, the lozenge has a moisture content of about 1.0% to about 5.0% w/w. In other embodiments, the lozenge has a moisture content of about 1.5% to about 4.5% w/w. In other embodiments, the lozenge has a moisture content of about 2.0% to about 4.0% w/w. In other embodiments, the lozenge has a moisture content of about 2.5% to about 3.5% w/w. In a particular embodiment, the moisture content is about 2.4% to about 4.4% w/w. In yet other embodiments, the lozenge has a moisture content of about 0.5, 1.0, 1.5, 2.0, 2.5, 3.0, 3.5, 4.0, 4.5, 5.5 or 6.0% w/w.

In some embodiments, the lozenge is formulated to mask the taste of memantine or memantine hydrochloride.

Rapid cough relief can be provided by both increasing the rate of absorption of memantine (e.g., by enhancing AUC₀₋₁, AUC₀₋₂, and Kₐ) and enhacing local demulcent effects (i.e., an agent that forms a soothing film over a mucous membrane) compared to conventional memantine compositions. One aspect of the present invention is to provide a memantine lozenge wherein the Tₘₐₓ for memantine after administration of the lozenge of the present invention is less than 8 hours, less than 7 hours, less than 6 hours, less than 5 hours, less than 4 hours, less than 3 hours, less than 2 hours, less than 1 hour, less than 45 minutes, less than 30 minutes, or less than 15 minutes, inclusive of all ranges therebetween. In some other embodiments, the Tₘₐₓ of memantine is about 15 min, about 30 min, about 45 min, about 1 hour, about 2 hours, about 3 hours, about 4 hours, about 5 hours, about 6 hours, about 7 hours, or about 8 hours, inclusive of all ranges therebetween.

In particular embodiments, the Tₘₐₓ of memantine after administration of the lozenge of the present invention ranges from about 15 minutes to about 2 hours, about 15 minutes to about 1 hour, about 30 minutes to about 2 hours, about 45 minutes to about 2 hours, about 1 hour to about 2 hours, about 1 hour to about 2.5 hours, about 1 hour to about 3 hours, about 1 hour to about 4 hours, or about 1 hour to about 6 hours, etc. In some other embodiments, the Tₘₐₓ of memantine after administration of the lozenge of the present invention ranges from about 2 hours to 2.5 hours, about 2 hours to about 3 hours, about 2 hours to about 3.5 hours, about 2 hours to about 4 hours, about 2 hours to about 4.5 hours, about 2 hours to about 5 hours, about 2 hours to about 5.5 hours, about 2 hours to about 6 hours. In other embodiments, Tₘₐₓ of memantine after administration of the lozenge of the present invention ranges from about 2.5 hours to about 3 hours, about 2.5 hours to about 3.5 hours, about 2.5 hours to about 4 hours, about 2.5 hours to about 4.5 hours, about 2.5 hours to about 5 hours, about 2.5 hours to about 5.5 hours, about 2.5 hours to about 6 hours. In one embodiment of the invention, the Tₘₐₓ of memantine after administration of the lozenge of the present invention ranges from about 3 hours to about 3.5 hours, about 3 hours to about 4 hours, about 3 hours to about 4.5 hours, about 3 hours to about 5 hours, about 3 hours to about 5.5 hours, or about 3 hours to about 6 hours. In another embodiment of the invention, the Tₘₐₓ of memantine after administration of the lozenge of the present invention ranges from about 3.5 hours to about 4 hours, 3.5 hours to about 4.5 hours, about 3.5 hours to about 5 hours, about 3.5 hours to about 5.5 hours, or about 3.5 hours to about 6 hours. In some embodiments of the invention, the Tₘₐₓ of memantine after administration of the compositions of the present invention ranges from about 4 hours to about 4.5 hours, about 4 hours to about 5 hours, about 4 hours to about 5.5 hours, or about 4 hours to about 6 hours.

In a particular embodiment of the invention, the Tₘₐₓ of memantine ranges between about 15 minutes to 30 minutes, about 15 minutes to about 45 minutes, about 15 minutes to about 1 hour, about 15 minutes to about 1.5 hours, about 15 minutes to about 2 hours or about 15 minutes to about 2.5 hours.

In another embodiment, the Tₘₐₓ of memantine ranges between about 2 minutes to about 3 hours, about 5 minutes to about 2 hours, about 10 minutes to about 1.5 hours, about 10 minutes to about 1 hour, and about 10 minutes to about 45 minutes. In another embodiment, the Tₘₐₓ of memantine ranges from about 30 minutes to about 6 hours, about 1 hour to about 6 hours, about 1.5 hours to about 6 hours, about 2 hours to about 5.5 hours, about 2.5 hours to about 5 hours, and about 2.5 hours to about 4 hours.

In another embodiment, the Tₘₐₓ of memantine ranges from about 30 minutes to about 6 hours, about 1 hour to about 6 hours, about 1.5 hours to about 6 hours, about 2 hours to about 5.5 hours, about 2.5 hours to about 5 hours, and about 2.5 hours to about 4 hours

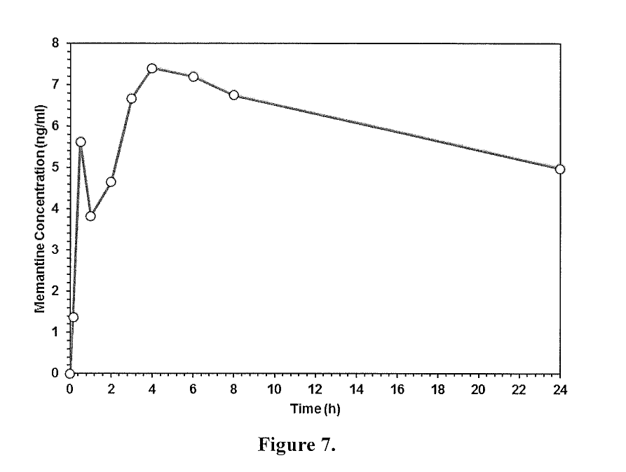

In another embodiment, the PK time/plasma concentration curve may have two or more “peaks.” For example, FIG. 7 (SUBJECT 2005) shows two peaks (Peak₁ and Peak₂) with a T₁ at about 15 min (C₁ at about 5.6 ng/mL) and a T₂ at about 4 hrs (C₂ at about 7.6 ng/mL). As stated above, the time/plasma concentration curve may have two or more peaks, wherein “Peak₁” is the first “peak” in time in the time/plasma concentration curve that provides a sharp or gradual increase in the concentration (y axis) over time (x axis), followed by a sharp or gradual decrease in the concentration over time in the time/plasma concentration curve; “Peak₂” is the second “peak” in time in the time/plasma concentration curve that provides a sharp or gradual increase in the concentration (y axis) over time (x axis), followed by a sharp or gradual decrease in the concentration over time in the time/plasma concentration curve. Accordingly, Peak₁ in the time/plasma concentration curve has a drug concentration of “C₁”, which refers to the maximum drug concentration within Peak₁; and a “T₁”, which refers to the time with the maximum concentration within Peak₁, i.e. the time of “C₁”. Accordingly, in a time/plasma concentration curve with at least two peaks, “C₂” in the time/plasma concentration curve refers to the maximum drug concentration within Peak₂; and “T₂” refers to the time with the maximum concentration within Peak₂, i.e. the time of “C₂”.

Accordingly, in one embodiment of the present invention, after a single buccal or sublingual administration to a patient, the lozenges of the present invention may provide a memantine T₁ ranging from about 2 minutes to about 3 hours, about 5 minutes to about 2 hours, about 10 minutes to about 1.5 hours, about 10 minutes to about 1 hour, and about 10 minutes to about 45 minutes.

In another embodiment of the present invention, after a single buccal or sublingual administration to a patient, the lozenges of the present invention may provide a memantine T₂ ranging from about 30 minutes to about 6 hours, about 1 hour to about 6 hours, about 1.5 hours to about 6 hours, about 2 hours to about 5.5 hours, about 2.5 hours to about 5 hours, and about 2.5 hours to about 4 hours.

In another embodiment, after a single buccal or sublingual administration to a patient, the lozenges of the present invention may provide a memantine T₁ ranging from about 2 minutes to about 3 hours and a memantine T₂ ranging from about 30 minutes to about 6 hours. In another embodiment, after a single buccal or sublingual administration to a patient, the lozenges of the present invention may provide a memantine T₁ ranging from about 5 minutes to about 2 hours and a memantine T₂ ranging from about 1 hour to about 6 hours. In another embodiment, after a single buccal or sublingual administration to a patient, the lozenges of the present invention may provide a memantine T₁ ranging from about 10 minutes to about 1.5 hours and a memantine T₂ ranging from about 1.5 hours to about 6 hours. In another embodiment, after a single buccal or sublingual administration to a patient, the lozenges of the present invention may provide a memantine T₁ ranging from about 10 minutes to about 1.5 hours and a memantine T₂ ranging from about 2 hours to about 5.5 hours. In another embodiment, after a single buccal or sublingual administration to a patient, the lozenges of the present invention may provide a memantine T₁ ranging from about 10 minutes to about 45 minutes and a memantine T₂ ranging from about 2.5 hours to about 4 hours T₁ ranging from about 10 minutes to about 1.5 hours and a memantine T₂ ranging from about 2 hours to about 5.5 hours.

In some embodiments of the invention, the elimination half-life (t_{1/2}) of the memantine in the present lozenge is less than about 80 hours, less than about 70 hours, less than about 65 hours, less than about 60 hours, less than about 55 hours, less than about 50 hours, less than about 45 hours, less than about 40 hours, less than about 35 hours, less than about 30 hours, less than about 25 hours, less than about 24 hours, less than about 22 hours, less than about 20 hours, less than about 18 hours, less than about 16 hours, or less than about 12 hours, inclusive of all ranges and subranges therebetween.

In some embodiments, the total clearance of the memantine in the present lozenge ranges from about 100 mL/min to about 250 mL/min. In some embodiments of the invention, total clearance of the memantine in present lozenge is more than about 180 mL/min, more than about 185 mL/min, more than about 190 mL/min, more than about 195 mL/min, or more than about 200 mL/min.

In another embodiment, after administration the lozenge of the present invention provides an AUC_{∞} for memantine of about 120 to about 18,000 ng-hr/mL, for example about 120, about 150, about 200, about 250, about 300, about 350, about 400, about 450, about 500, about 550, about 600, about 650, about 700, about 750, about 800, about 850, about 900, about 950, about 1000, about 1100, about 1200, about 1300, about 1400, about 1500, about 1600, about 1700, about 1800, about 1900, about 2000, about 2200, about 2400, about 2600, about 2800, about 3000, about 3200, about 3400, about 3600, about 3800, about 4000, about 4200, about 4400, about 4600, about 4800, about 5000, about 5200, about 5400, about 5600, about 5800, about 6000, about 6200, about 6400, about 6600, about 6800, about 7000, about 7200, about 7400, about 7600, about 7800, about 8000, about 8200, about 8400, about 8600, about 8800, about 9000, about 9200, about 9400, about 9600, about 9800, about 10,000, about 10,500, about 11,000, about 11,500, about 12,000, about 12,500, about 13,000, about 13,500, about 14,000, about 14,500, about 15,000, about 15,500, about 16,000, about 16,500, about 17,000, about 17,500, or about 18,000 ng-hr/mL, inclusive of all ranges and subranges therebetween.

In another embodiment, after administration the lozenge of the present invention provides an AUC₀₋₁ (AUC in the first hour after administration) for memantine of about 1 to about 15 ng-hr/mL, for example about 1, about 2, about 3, about 4, about 5, about 6, about 7, about 8, about 9, about 10, about 11, about 12, about 13, about 14, or about 15 ng-hr/mL, inclusive of all ranges and subranges therebetween.

In another embodiment, after administration the lozenge of the present invention provides an AUC₀₋₂ (AUC in the first two hours after administration) for memantine of about 5 to about 30 ng-hr/mL, for example about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, or about 30 ng-hr/mL, inclusive of all ranges and subranges therebetween.

In various embodiments, after administration of the memantine-containing lozenge of the present invention, the Cₘₐₓ of memantine ranges (after single administration) from about 5 ng/mL to about 50 ng/mL, for example about 5 ng/mL, about 6 ng/mL, about 7 ng/mL, about 8 ng/mL, about 9 ng/mL, about 10 ng/mL, about 11 ng/mL, about 12 ng/mL, about 13 ng/mL, about 14 ng/mL, about 15 ng/mL, about 16 ng/mL, about 17 ng/mL, about 18 ng/mL, about 19 ng/mL, about 20 ng/mL, about 21 ng/mL, about 22 ng/mL, about 23 ng/mL, about 24 ng/mL, about 25 ng/mL, about 26 ng/mL, about 27 ng/mL, about 28 ng/mL, about 29 ng/mL, about 30 ng/mL, about 31 ng/mL, about 32 ng/mL, about 33 ng/mL, about 34 ng/mL, about 35 ng/mL, about 36 ng/mL, about 37 ng/mL, about 38 ng/mL, about 39 ng/mL, about 40 ng/mL, about 41 ng/m, about 42 ng/mL, about 43 ng/mL, about 44 ng/mL, about 45 ng/mL, about 46 ng/mL, about 47 ng/mL, about 48 ng/mL, about 49 ng/mL, or about 50 ng/mL, inclusive of all ranges therebetween.

The present inventors have found that in the lozenges of the present invention, both Cₘₐₓ and AUC are dose proportional. Thus, in some embodiments, for memantine lozenges of the present invention, the dose normalized oral or buccal Cₘₐₓ (normalized to a 1 mg dose) ranges from about 1 ng/mL to about 2 ng/mL, for example about 1, about 1.1, about 1.2, about 1.3, about 1.4, about 1.5, about 1.6, about 1.7, about 1.8, about 1.9, or about 2 ng/mL (per 1 mg dose), inclusive of all ranges and subranges therebetween.

In another embodiment, after administration the lozenge of the present invention provides a Ka (absorption rate constant) of about 0.1 h⁻¹ to about 10 h⁻¹. In another embodiment, the Ka may be about 0.3 to about 7.0, about 0.4 to about 6.9, about 1.5 to about 2.0, about 6.5 to about 7.0, about 0.2, about 0.3, about 0.4, about 0.5, about 1.0, about 1.5, about 2.0. about 2.5, about 3.0, about 3.5, about 4.0, about 4.5, about 5.0, about 5.5, about 6.0, about 6.5, and about 7.0 h⁻¹.

In another embodiment, after administration the lozenge of the present invention provides an absorption half-life (T_{1/2-absorption}) of about 0.04 hr to about 2.8 hr. In another embodiment, T_{1/2-absorption} may be about 0.1 hrs, 0.2 hrs, 0.3 hrs, 0.4 hrs, 0.5 hrs, 0.6 hrs, 0.7 hrs, 0.8 hrs, 0.9 hrs, 1.0 hrs, 1.1 hrs, 1.2 hrs, 1.3 hrs, 1.4 hrs, 1.5 hrs, 1.6 hrs, 1.7 hrs, 1.8 hrs, 1.9 hrs, 2.0 hrs, 2.1 hrs, 2.2 hrs, 2.3 hrs, 2.4 hrs, 2.5 hrs, 2.6 hrs, 2.7 hrs and 2.8 hrs.

### Candy Lozenges

In some embodiments, the lozenge is a candy lozenge. In a specific embodiment, the candy lozenge may comprise memantine. In some embodiments, the memantine is memantine hydrochloride (3,5-dimethyl-1-adamantanamine hydrochloride).

In a specific embodiment, the candy lozenge comprising memantine may be used as an antitussive to provide symptomatic treatment of cough, specifically as a lozenge. As described above, the inclusion of an alkalinizing agent to the lozenge allows for an increase in the pH of the oral cavity, thus reducing the ionization of memantine in the oral cavity, thereby improving passive absorption of memantine in the oral mucosa. Accordingly, one aspect of the invention relates to a candy lozenge comprising: memantine; and an alkalinizing agent.

The combination of memantine and an alkalinizing agent, however, may not be compatible, resulting in degradation of the memantine or precipitation of the memantine from solution during the preparation of the lozenge. Indeed, the mixture of the memantine and an alkalinizing agent during preparation of the lozenge may result in about 40% to about 60% of the memantine drug being degraded. The rate of degradation of memantine during preparation of the lozenge may increase with an in increase in the pH due to the addition of the alkalinizing agent. Further, the reaction between memantine and the alkalinizing agent may be increased by the moisture content of the candy lozenge composition, or individual components of the candy lozenge composition. Accordingly, some embodiments provide a candy lozenge composition with a low moisture content so that combinations of the memantine and alkalinizing agent do not result in degradation of the memantine under preparation or storage conditions. In particular embodiments, the composition has a moisture content of less than about 5%, less than about 4%, less than about 3%, less than about 2%, or less than about 1%, inclusive of all ranges therebetween.

In one embodiment of the present invention, a substantial amount of memantine and a substantial amount of the alkalinizing agent may be physically or chemically separated in the lozenge. The physical separation may include for example, the alkalinizing agent and the memantine being in different compartments of the lozenge. In another embodiment, the physical separation may include the alkalinizing agent and the memantine being in separate layers of the lozenge. In another embodiment, the physical separation may include minimal contact of the alkalinizing agent and the memantine wherein the alkalinizing agent and the memantine are in different compartments of the lozenge. In one embodiment, the minimal physical contact of the alkalinizing agent and the memantine may occur where the alkalinizing agent compartment and the memantine compartment meet in the lozenge. For example, the lozenge may include a bilayer, wherein the memantine compartment is on one side of the lozenge and the alkalinizing agent is on another side of the lozenge. Accordingly, the surface area of where the memantine compartment and the alkalinizing agent compartment come into contact is minimized.

The rate of browning (caused by decomposition of components of the candy lozenge during processing) may increase with an increase in pH due to the addition of the alkalinizing agent. The browning may lead to a bitter burnt taste. Accordingly, some embodiments provide a candy lozenge without the bitter burnt taste that still allows for the increase of memantine buccal/oral mucosal absorption by reducing its ionization.

In one embodiment of the present invention, the memantine and alkalinizing agent are not in contact with each other in the lozenge, thereby reducing the degradation of memantine. In another embodiment, substantially all of the memantine and alkalinizing agent are in different layers of the candy lozenge. In some embodiments, the candy lozenge comprises two or more layers, wherein all or substantially all of the memantine is in a first layer, and all or substantially all of the alkalinizing agent is in a second layer. In a specific embodiment, the first layer is an inner layer comprising substantially all of the memantine, and the second layer is an outer layer, disposed over the inner layer comprising substantially all of the alkalinizing agent. In other embodiments, the first layer is an outer layer comprising substantially all of the memantine, and the second layer is an inner layer comprising substantially all of the alkalinizing agent. In certain embodiments, the candy lozenge is a bilayer, wherein all or substantially all of the memantine is in a first layer, and all or substantially all of the alkalinizing agent is in a second layer. In yet other embodiments, one or more of the memantine and alkalinizing agent are each apportioned into multiple different layers. For example, the total memantine dose can be divided into two or more different layers and the total amount of alkalinizing agent can be divided into two or more different layers; the total memantine dose can be entirely contained in one layer, while the alkalinizing agent is divided into two or more different layers; or the total memantine dose is divided into two or more different layers and the alkalinizing agent is entirely contained in one layer. In some embodiments the memantine and alkalinizing agent-containing layers are disposed directly on each other (e.g., and alkalinizing agent-containing layer is disposed directly onto a memantine-containing layer), while in other embodiments one or more other layers are disposed between the memantine-containing layers and alkalinizing agent-containing layers. The first and second layers can be arranged in any manner, for example in a core/shell arrangement (e.g., where the first layer is the core and the second layer is a shell surrounding the core, or vice versa), or the first and second layers can be arranged as a bilayer in which one side of the lozenge comprises the first layer, and the opposing side of the lozenge comprises the second layer.

In still other embodiments, the memantine and alkalinizing agent are not dispersed in separate layers, but rather in separate phases within the lozenge. For example, one or more memantine-containing phases can be distributed within a lozenge matrix, wherein the alkalinizing agent is dispersed within the matrix. Alternatively, one or more alkalinizing agent phases can be distributed within a lozenge matrix, wherein the memantine is dispersed within the matrix.

The addition of an alkalinizing agent to a candy lozenge may have other undesirable effects. For example, the addition of an alkalinizing agent during the preparation of a candy lozenge may cause a “browning” reaction, which may lead to a slightly bitter “burnt” taste of the lozenge that could adversely affect patient compliance. Specifically, in the preparation of a candy lozenge, the addition of an alkalinizing agent may react with a reducing sugar as a Maillard reaction. Further, various studies have demonstrated an increase in reaction rate with a rise in pH. The relationship between the reaction rate and pH would therefore render those foods/candies of high alkalinity more susceptible to this reaction. Accordingly, the addition of an alkalinizing agent or an attempt to increase the pH of a candy lozenge comprising memantine presents numerous challenges.

In one embodiment, a non-reducing carbohydrate, sugar or sugar-substitute may be added as the sweetener to the lozenge. In one specific embodiment, isomalt, a non-reducing sugar substitute may be added as one or more of the sweeteners to the candy lozenge. Indeed, isomalt is a sugar-free lozenge base, tends to be less reactive with excipients, demonstrates a good stability profile and has a high glass transition temperature which will allow the formula to be heated to sufficient temperatures for mixing lozenge ingredients. Unexpectedly, however, the production of isomalt also ensures numerous impurities in any lot of isomalt. Although manufacturers may attempt to limit the reducing sugar content in their batches, it is very difficult to completely eliminate them.

In another embodiment, other non-reducing sugars or sugar substitutes may be included in the candy lozenge. In one embodiment, sorbitol may be included in the candy lozenge as a sugar substitute. Sorbitol, however, as with other non-reducing sugars and sugar substitutes, is not conducive for a hard candy lozenge environment that would be desirable for an oral dosage form for treating cough. For example, sorbitol lozenges are not as hard as the isomalt based candy lozenges and they take much longer to cure (˜24 hours). This longer curing time makes this process much less scalable when larger batches need to be made at faster speeds. In other words, sorbitol lozenges are too soft for the desired dosage from and take too long to manufacture. Accordingly, pharmaceutical dosage forms, such as lozenges, wherein the pH needs to be high, create numerous issues in their development. Novel lozenge dosage forms and/or methods of making these lozenge dosage forms, specifically, where the pH of the lozenges needs to be high, are thus desired.

In one embodiment of the present invention, a substantial amount of the carbohydrate, sugar or sugar substitute may be included in the compartment of the lozenge that comprises a substantial amount of memantine. In other words, a substantial amount of the carbohydrate, sugar or sugar substitute may be physically and/or chemically separated from the alkalinizing agent. In another embodiment, the lozenge may include a bilayer, wherein a substantial amount of the carbohydrate, sugar or sugar substitute is included in the memantine layer on one side of the lozenge and the alkalinizing agent layer is on another side of the lozenge.

In another embodiment, substantially all of the carbohydrate, sugar or sugar substitute and alkalinizing agent are in different layers of the candy lozenge. In some embodiments, the candy lozenge comprises two or more layers, wherein all or substantially all of the carbohydrate, sugar or sugar substitute is in a first layer, and all or substantially all of the alkalinizing agent is in a second layer. In a specific embodiment, the first layer is an inner layer comprising substantially all of the carbohydrate, sugar or sugar substitute, and the second layer is an outer layer, disposed over the inner layer comprising substantially all of the alkalinizing agent. In other embodiments, the first layer is an outer layer comprising substantially all of the carbohydrate, sugar or sugar substitute, and the second layer is an inner layer comprising substantially all of the alkalinizing agent. In a specific embodiment, the layer comprising substantially all of the carbohydrate, sugar or sugar substitute may also comprise memantine. In yet other embodiments, one or more of the memantine, the carbohydrate, sugar or sugar substitute and alkalinizing agent are each apportioned into multiple different layers. In still other embodiments, the carbohydrate, sugar or sugar substitute and alkalinizing agent are not dispersed in separate layers, but rather in separate compartments or phases within the lozenge. For example, one or more carbohydrate, sugar or sugar substitute containing phases can be distributed within a lozenge matrix, wherein the alkalinizing agent is dispersed within the matrix. Alternatively, one or more alkalinizing agent phases can be distributed within a lozenge matrix, wherein the carbohydrate, sugar or sugar substitute is dispersed within the matrix. In another specific embodiment, memantine may also be included in separate compartments from the carbohydrate, sugar or sugar substitute and alkalinizing agent.

Patient compliance, however, often requires that the carbohydrate, sugar or sugar substitute, i.e., the sweetening agents or flavorant that provides for a pleasant candy like taste in the lozenge, be homogeneously spread throughout the lozenge. Indeed, a candy lozenge wherein the carbohydrate, sugar or sugar substitute are substantially removed from one compartment of the dosage form may cause an unpleasant and undesirable bitter taste. Accordingly, in one embodiment of the present invention, a browning reaction inhibitor may be added to the candy lozenges of the present invention. In a specific embodiment, browning reaction inhibitor may be reducing agents, chelating agents, citric acid, phosphoric acid, cyclodextrins, aromatic enzyme inhibitors, chitosan, peptides, carbohydrate derivatives, proteolytic enzymes, and agents capable of inhibiting either chemical degradation of memantine or browning of the memantine and alkalinizing agent mixture. In a specific embodiment, the browning reaction inhibitor may be a sulfite, ascorbic acid, glutathione and/or cysteine. In another specific embodiment, the browning reaction inhibitor may be sodium metabisulfite (SMBS).

The candy lozenges of the present invention may also include compounds that complex with or substantially reduce the ionized memantine. The addition of, for example an ionic exchange resin, may substantially reduce the ionized memantine. In a specific embodiment, the candy lozenges of the present invention may comprise one or more ion exchange resins. In some embodiments, the ion exchange resin may be a cation exchange resin. In some embodiments, a substantial amount of the ion exchange resin may be included in the compartment or layer of the lozenge that comprises a substantial amount of memantine. In some embodiments, the first layer further comprises one or more ion exchange resins.

In some embodiments, the memantine is present in the candy lozenge at about 1 mg to about 20 mg, for example about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, inclusive of all ranges and subranges therebetween. In another embodiment, the memantine is present in the candy lozenge at about 3 mg to about 18 mg. In another embodiment, the memantine is present in the candy lozenge at about 5 mg to about 16 mg. In another embodiment, the memantine is present in the candy lozenge at about 7 mg to about 16 mg. In another embodiment, the memantine is present in the candy lozenge at about 9 mg to about 14 mg. In another embodiment, the memantine is present in the candy lozenge at about 6 mg to about 9 mg. In another embodiment, the memantine is present in the candy lozenge at about 5 mg to about 7 mg. In another embodiment, the memantine is present in about 0.1 to 6 percent by weight of the first layer. In another embodiment, the memantine is present in about 0.1 to 6 percent by weight of the candy lozenge. In certain embodiments, the amount of the memantine in the first layer is about 6 mg to about 8 mg, about 7 mg to about 9 mg, about 7 mg to about 8 mg, about 7 mg, or about 8 mg. In particular embodiments, the amount of the memantine is about 7 mg to about 8 mg. In further embodiments, the amount of the memantine is about 7.5 mg. In further embodiments, the amount of the memantine is about 6.0 mg.

In another embodiment, the memantine is present at about 0.07 to 4 percent by weight of the first layer of the candy lozenge. In another embodiment, the amount of memantine is about 0.1 to 3 percent by weight of the first layer of the candy lozenge. In another embodiment, the amount of memantine is about 0.1 to 1.5 percent by weight of the first layer of the candy lozenge. In certain embodiments, the amount of the memantine is about 0.1 to 0.3 percent, about 0.2 to 0.4 percent, about 0.2 to 0.3 percent, about 0.2 percent, or about 0.3 percent by weight of the first layer of the candy lozenge. In other embodiments, the amount of the memantine is about 0.2 to 0.3 percent by weight of the first layer of the candy lozenge. In various embodiments, the amount of memantine is about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0 percent, about 3.1 percent, about 3.2 percent, about 3.3 percent, about 3.4 percent, about 3.5 percent, about 3.6 percent, about 3.6 percent, about 3.7 percent, about 3.8 percent, about 3.9 percent, or about 4.0 percent of the first layer of the candy lozenge, inclusive of all values and ranges therebetween.

In another embodiment, the amount of memantine is about 0.07 to 0.4 percent by weight of the first layer. In certain embodiments, the amount of the memantine is about 0.1 to 0.3 percent, about 0.2 to 0.4 percent, about 0.2 to 0.3 percent, about 0.2 percent, or about 0.3 percent by weight of the first layer. In other embodiments, the amount of the memantine is about 0.2 to 0.3 percent by weight of the first layer. In yet other embodiments, the amount of the memantine in the first layer is about 2.4 percent by weight of the first layer.

In another embodiment, the memantine is present at about 0.07 to 4 percent by weight of the candy lozenge. In another embodiment, the amount of memantine is about 0.1 to 3 percent by weight of the candy lozenge. In another embodiment, the amount of memantine is about 1 to 2 percent by weight of the candy lozenge. In another embodiment, the amount of memantine is about 0.1 to 1.5 percent by weight of the candy lozenge. In certain embodiments, the amount of the memantine is about 0.1 to 0.3 percent, about 0.2 to 0.4 percent, about 0.2 to 0.3 percent, about 0.2 percent, or about 0.3 percent by weight of the candy lozenge. In other embodiments, the amount of the memantine is about 0.2 to 0.3 percent by weight of the candy lozenge. In various embodiments, the amount of memantine is about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0 percent, about 3.1 percent, about 3.2 percent, about 3.3 percent, about 3.4 percent, about 3.5 percent, about 3.6 percent, about 3.6 percent, about 3.7 percent, about 3.8 percent, about 3.9 percent, about 4.0 percent of the candy lozenge, inclusive of all values and ranges therebetween.

In some embodiments, the alkalinizing agent is present in the candy lozenge at about 1 mg to about 40 mg. In another embodiment, the alkalinizing agent is present in the candy lozenge at about 5 mg to about 35 mg. In another embodiment, the alkalinizing agent is present in the candy lozenge at about 10 mg to about 30 mg. In another embodiment, the alkalinizing agent is present in the candy lozenge at about 15 mg to about 25 mg. In another embodiment, the alkalinizing agent is present in the candy lozenge at about 4 mg to about 9 mg. In another embodiment, the alkalinizing agent is present in the candy lozenge at about 5 mg to about 8 mg. In another embodiment, the alkalinizing agent is present in the candy lozenge at about 6 mg to about 7 mg. In another embodiment, the alkalinizing agent is present in the second layer.

In some embodiments, two or more alkalinizing agents are present in the candy lozenge at about 1 mg to about 40 mg. In another embodiment, the two or more alkalinizing agents are present in the candy lozenge at about 5 mg to about 35 mg. In another embodiment, the two or more alkalinizing agents are present in the candy lozenge at about 10 mg to about 30 mg. In another embodiment, the two or more alkalinizing agents are present in the candy lozenge at about 15 mg to about 25 mg. In another embodiment, the two or more alkalinizing agents are present in the candy lozenge at about 5 mg to about 15 mg. In another embodiment, the two or more alkalinizing agents are present in the candy lozenge at about 20 mg to about 40 mg.

In certain embodiments, the lozenge has a first layer comprising memantine and a second layer comprising the alkalinizing agent. The amount of the alkalinizing agent in the second layer is about 5 to 7 mg, 6 to 8 mg, 6 to 7 mg, 6 mg or 7 mg. In particular embodiments, the amount of the alkalinizing agent is about 6 to 7 mg. In further embodiments, the amount of the alkalinizing agent is about 6.7 mg. In certain embodiments, the amount of the alkalinizing agent is about 0.1 to 0.4 percent, about 0.1 to 0.3 percent, about 0.2 to 0.4 percent, about 0.2 to 0.3 percent, about 0.2 percent, or about 0.3 percent by weight of the second layer. In other embodiments, the amount of the alkalinizing agent is about 0.2 to 0.3 percent by weight of the second layer. In yet other embodiments, the amount of the memantine in the first layer is about 2.4 percent by weight of the second layer.

In another embodiment of the present invention, the alkalinizing agent included in the candy lozenge may be aluminum carbonate, aluminum hydroxide, ammonium carbonate, ammonium solution, calcium carbonate, calcium phosphate, diethanolamine, magnesium carbonate, magnesium hydroxide, magnesium oxide, magnesium trisilicate, monoethanolamine, potassium bicarbonate, potassium carbonate, potassium citrate, potassium hydroxide, sodium acetate, sodium bicarbonate, sodium carbonate, sodium citrate, sodium hydroxide, sodium phosphate dibasic, sodium phosphate monobasic, sodium phosphate tribasic, triethanolamine, tromethane, and combinations thereof. In some embodiments, the alkalinizing agent is magnesium oxide, potassium carbonate, sodium phosphate tribasic, sodium carbonate, sodium hydroxide and combinations thereof. In other embodiments, the alkalinizing agent is sodium carbonate and/or sodium hydroxide. In yet other embodiments, the alkalinizing agent is sodium hydroxide. In another specific embodiment, the alkalinizing agent is sodium carbonate.

In another specific embodiment, the alkalinizing agent may be a buffering agent, such as an alkaline buffering agent. Alkaline buffering agents are mixtures of weak bases and their conjugate acid(s), such as, for example, sodium carbonate/sodium bicarbonate, barbitone sodium/hydrochloric acid, trisaminomethane/hydrochloric acid, sodium tetraborate/hydrochloric acid, glycine/sodium hydroxide, sodium carbonate/sodium hydrogen carbonate, sodium tetraborate/sodium hydroxide, sodium bicarbonate/sodium hydroxide, sodium hydrogen orthophosohate/sodium hydroxide, and potassium chloride/sodium hydroxide. As stated above, the inclusion of an alkalinizing agent such as an alkaline buffering agent to the lozenge allows for an increase in the pH, thus reducing the ionization of memantine in the oral cavity, and thereby improving passive absorption of memantine in the oral mucosa. In addition, buffering agents resist pH changes. Accordingly, adding an alkaline buffering agent may provide more control over the pH in the oral cavity, and thus provide more consistent absorption and consistent pharmacokinetics. In another specific embodiment, the alkalinizing agent is an alkaline buffering agent such as sodium carbonate and sodium bicarbonate.

In another embodiment, the alkalinizing agent present in the second layer is sodium hydroxide. In a specific embodiment, sodium hydroxide is present in the amount of about 4 mg to about 8 mg. In another embodiment, sodium hydroxide is present in the amount of about 5 mg to about 7 mg. In another embodiment, sodium hydroxide is present in the amount of about 6 mg to about 8 mg. In another embodiment, sodium hydroxide is present in the amount of about 6 mg to about 8 mg.

In another embodiment, the alkalinizing agent present in the second layer is sodium carbonate. In a specific embodiment, sodium carbonate is present in the amount of about 1 mg to about 35 mg. In another embodiment, sodium carbonate is present in the amount of about 3 mg to about 25 mg. In another embodiment, sodium carbonate is present in the amount of about 5 mg to about 15 mg. In another embodiment, sodium carbonate is present in the amount of about 7.5 mg to about 12 mg.

In some embodiments, the candy lozenge may comprise one or more pharmaceutically acceptable excipients independently selected from sweetening agents, such as a carbohydrate, sugar or sugar substitute, reducing agents, colorants, flavorants, permeation enhancers, solvents, co-solvents and diluents. In other embodiments, the one or more pharmaceutically acceptable excipients may comprise menthol. In yet other embodiments, the one or more pharmaceutically acceptable excipients may comprise the carbohydrate, sugar or sugar substitute isomalt. In certain embodiments, the one or more pharmaceutically acceptable excipients are isomalt and menthol.

In further embodiments, candy lozenges of the present invention may include the pharmaceutically acceptable excipient isomalt, and a browning reaction inhibitor. In another embodiment, candy lozenges of the present invention may include the pharmaceutically acceptable excipient isomalt, and the browning reaction inhibitor, SMBS.

In another embodiment of the present invention, the candy lozenge may comprise compartments or layers with various ingredients, including one or more pharmaceutically acceptable excipients, memantine, one or more browning reaction inhibitors and one or more alkalinizing agents. In one embodiment, the candy lozenge may compartments or layers wherein the various ingredients, including one or more pharmaceutically acceptable excipients, memantine, one or more browning reaction inhibitors and one or more alkalinizing agents, vary.

In some embodiments, the candy lozenge comprises: a first layer further comprising memantine, isomalt, menthol, acesulfame potassium, black cherry flavor and mineral oil; and a second layer further comprising an alkalinizing agent, isomalt and sodium metabisulfite (SMBS).

In some embodiments, the candy lozenge further comprises one or more coating agents. In particular embodiments, the first layer, the second layer, or both the first layer and the second layer further comprise one or more coating agents. In certain embodiments, the one or more coating agents encapsulate the memantine. In other embodiments, the one or more coating agents encapsulate the alkalinizing agent. In yet other embodiments, the one or more coating agents encapsulate both the memantine and the alkalinizing agent.

### Compressed Lozenges

In another embodiment, the lozenge may be in a form wherein a substantial amount of memantine and a substantial amount of the alkalinizing agent are chemically separated in the lozenge. In a specific embodiment, the lozenge may be manufactured and prepared wherein memantine and the alkalinizing agent are not in a liquid state and thus, do not chemically react, or are substantially inhibited from chemically reacting. In one embodiment, the memantine and the alkalinizing agent in the lozenges of the present invention may be in separate granules. In another embodiment, the granules may be coated. In some embodiments, the lozenge is a compressed lozenge. In a specific embodiment, the compressed lozenge may comprise memantine. In some embodiments, the memantine is memantine hydrochloride (3,5-dimethyl-1-adamantanamine hydrochloride). In another specific embodiment, the compressed lozenge comprises memantine and an alkalinizing agent. In some embodiments, the memantine exists as memantine free base after having been converted from memantine HCl during the granulation solution preparation. A compressed lozenge, for example, allows for the lozenge to be manufactured in a semi-solid or solid state and thus limits the interaction between memantine and the alkalinizing agent. Accordingly, a compressed lozenge may thus still allow for the memantine and the alkalinizing agent to be physically separated.

In another specific embodiment, the compressed lozenge allows for the ingredients to be in a solid state throughout the manufacturing process, thereby reducing the degradation of memantine due to the addition of an alkalinizing agent. In another specific embodiment, the memantine and alkalinizing agent are not in contact with each other in the compressed lozenge. In another specific embodiment, substantially all of the memantine and alkalinizing agent are physically separated in different components or compartments of the compressed lozenge. In another embodiment, substantially all of the memantine and alkalinizing agent are in different granules that are blended and then compressed into a lozenge. In another embodiment, substantially all of the memantine and alkalinizing agent are in pre-mixed in a granulation solution or suspension and then granulated with dry powder ingredients of the compressed lozenge. In another embodiment, substantially all of the memantine and alkalinizing agent are in different layers of the compressed lozenge. In some embodiments, the lozenge further comprises, in addition to the memantine and the alkalinizing agent, one or more pharmaceutically acceptable excipients independently selected from sweetening agents, colorants, flavorants, permeation enhancers, solvents, co-solvents, fillers, binders, disintegrants, lubricants, glidants and moisture scavengers. In certain embodiments, the one or more pharmaceutically acceptable excipients are independently selected from isomalt, acesulfame potassium, povidone, microcrystalline cellulose, magnesium aluminometasilicate, polyethylene glycol 8000 and sodium stearyl fumarate. In other embodiments, the one or more pharmaceutically acceptable excipients comprise isomalt. In yet other embodiments, the one or more pharmaceutically acceptable excipients comprise isomalt and acesulfame potassium.

In some embodiments, the compressed lozenge is about 0.1 g to about 2 g in weight. In some embodiments, the compressed lozenge is about 0.1 g to about 0.5 g in weight. In one embodiment, the compressed lozenge is about 0.2 g to about 1.0 g in weight. In another embodiment, the compressed lozenge is about 0.1 g in weight. In another embodiment, the compressed lozenge is about 0.15 g in weight. In another embodiment, the compressed lozenge is about 0.2 g in weight. In another embodiment, the compressed lozenge is about 0.25 g in weight. In another embodiment, the compressed lozenge is about 0.3 g in weight. In another embodiment, the compressed lozenge is about 0.35 g in weight. In another embodiment, the compressed lozenge is about 0.4 g in weight. In another embodiment, the compressed lozenge is about 0.45 g in weight. In another embodiment, the compressed lozenge is about 0.5 g in weight.

In some embodiments, the compressed lozenge is about 0.5 g to about 5 g in weight. In one embodiment, the compressed lozenge is about 0.5 g to about 4.5 g in weight. In another embodiment the compressed lozenge is about 1.5 g to about 4.5 g in weight. In another embodiment, the compressed lozenge is about 2 g to about 4 g in weight. In another embodiment, the compressed lozenge is about 2.5 g to about 3.5 g in weight. In certain embodiments, the compressed lozenge weighs about 0.5 g. In other embodiments, the compressed lozenge weighs about 1 g. In other embodiments, the compressed lozenge weighs about 1.5 g. In other embodiments, the compressed lozenge weighs about 2 g. In other embodiments, the compressed lozenge weighs about 2.5 g. In other embodiments, the compressed lozenge weighs about 3 g. In other embodiments, the compressed lozenge weighs about 3.5 g. In other embodiments, the compressed lozenge weighs about 3.5 g. In other embodiments, the compressed lozenge weighs about 4 g. In other embodiments, the compressed lozenge weighs about 4.5 g. In yet other embodiments, the compressed lozenge weighs about 4.75 g. In further embodiments, the compressed lozenge weighs about 5 g. In some embodiments, the memantine is present in the compressed lozenge at about 1 mg to about 20 mg, for example about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg, inclusive of all ranges and subranges therebetween. In another embodiment, the memantine is present in the compressed lozenge at about 3 mg to about 18 mg. In another embodiment, the memantine is present in the compressed lozenge at about 5 mg to about 16 mg. In another embodiment, the memantine is present in the compressed lozenge at about 7 mg to about 16 mg. In another embodiment, the memantine is present in the compressed lozenge at about 9 mg to about 14 mg. In another embodiment, the memantine is present in the compressed lozenge at about 6 mg to about 9 mg. In certain embodiments, the amount of the memantine is about 6 mg to about 8 mg, about 7 mg to about 9 mg, about 7 mg to about 8 mg, about 7 mg, or about 8 mg. In particular embodiments, the amount of memantine is about 7 mg to about 8 mg. In another embodiment, the amount of memantine is about 5 mg to about 7 mg. In further embodiments, the amount of the memantine is about 7.5 mg. In another embodiment, the amount of the memantine is about 6.0 mg.

In another embodiment, the memantine is present at about 0.01 to about 20 percent by weight of the lozenge, including about 0.1%, about 0.2%, about 0.3%, about 0.4%, about 0.5%, about 0.6%, about 0.7%, about 0.8%, about 0.9%, about 1%, about 1.2%, about 4%, about 1.6%, about 1.8%, about 2.0%, about 2.2%, about 2.4%, about 2.6%, about 2.8%, about 3.0%, about 3.2%, about 3.4%, about 3.6%, about 3.8%, about 4.0%, about 4.2%, about 4.4%, about 4.6%, about 4.8%, about 5.0%, about 5.2%, about 5.4%, about 5.6%, about 5.8%, about 6.0%, about 6.2%, about 6.4%, about 6.6%, about 6.8%, about 7.0%, about 7.2%, about 7.4%, about 7.6%, about 7.8%, about 8.0%, about 8.2%, about 8.4%, about 8.6%, about 8.8%, about 9.0%, about 9.2%, about 9.4%, about 9.6%, about 9.8%, about 10.0%, about 10.2%, about 10.4%, about 10.6%, about 10.8%, about 11.0%, about 11.2%, about 11.4%, about 11.6%, about 11.8%, about 12.0%, about 12.2%, about 12.4%, about 12.6%, about 12.8%, about 13.0%, about 13.2%, about 13.4%, about 13.6%, about 13.8%, about 14.0%, about 14.2%, about 14.4%, about 14.8%, about 15.0%, about 15.2%, about 15.4%, about 15.6%, about 15.8%, about 16.0%, about 16.2%, about 16.4%, about 16.6%, about 16.8%, about 17.0%, about 17.2%, about 17.4%, about 17.6%, about 17.8%, about 18.0%, about 18.2%, about 18.4%, about 18.6%, about 18.8%, about 19.0%, about 192%, about 19.4%, about 19.6%, about 19.8%, or about 20.0%, inclusive of all ranges therebetween.

In another embodiment, the memantine is present at about 0.01 to about 10 percent by weight of the lozenge. In another embodiment, the memantine is present at about 1.0 to 10 percent by weight of the lozenge. In another embodiment, the memantine is present at about 2.0 to about 8.0 percent by weight of the lozenge. In another embodiment, the memantine is present at about 5.0 to about 8.0 percent by weight of the lozenge. In another embodiment, the memantine is present at about 6.0 to about 8.0 percent by weight of the lozenge. In another embodiment, the amount of memantine is about 0.1 to about 3.0 percent by weight of the lozenge. In another embodiment, the amount of memantine is about 0.1 to about 1.5 percent by weight of the lozenge. In another embodiment, the amount of memantine is about 1 to about 2 percent by weight of the lozenge. In certain embodiments, the amount of the memantine is about 0.1 to about 0.3 percent, about 0.2 to about 0.4 percent, about 0.2 to about 0.3 percent, about 0.2 percent, or about 0.3 percent by weight of the lozenge. In various embodiments, the amount of memantine is about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0 percent, about 3.1 percent, about 3.2 percent, about 3.3 percent, about 3.4 percent, about 3.5 percent, about 3.6 percent, about 3.6 percent, about 3.7 percent, about 3.8 percent, about 3.9 percent, about 4.0 percent, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, or about 10.0, by percent weight of the lozenge.

In some embodiments, the alkalinizing agent is present in the compressed lozenge at about 1 mg to about 40 mg. In another embodiment, the alkalinizing agent is present in the lozenge at about 5 mg to about 35 mg. In another embodiment, the alkalinizing agent is present in the lozenge at about 10 mg to about 30 mg. In another embodiment, the alkalinizing agent is present in the lozenge at about 15 mg to about 25 mg. In another embodiment, the alkalinizing agent is present in the lozenge at about 4 mg to about 9 mg. In another embodiment, the alkalinizing agent is present in the lozenge at about 5 mg to about 8 mg. In another embodiment, the alkalinizing agent is present in the lozenge at about 6 mg to about 7 mg.

In some embodiments, two or more alkalinizing agents are present in the lozenge at a total weight of about 1 mg to about 40 mg. In another embodiment, the two or more alkalinizing agents are present in the lozenge at a total weight of about 5 mg to about 35 mg. In another embodiment, the two or more alkalinizing agents are present in the lozenge at a total weight of about 10 mg to about 30 mg. In another embodiment, the two or more alkalinizing agents are present in the lozenge at a total weight of about 15 mg to about 25 mg. In another embodiment, the two or more alkalinizing agents are present in the lozenge at a total weight of about 5 mg to about 15 mg. In another embodiment, the two or more alkalinizing agents are present in the lozenge at a total weight of about 20 mg to about 40 mg.

In certain embodiments, the amount of the alkalinizing agent in the lozenge is about 5 to about 50 mg, about 5 mg to about 40 mg, about 7 mg to 7 about 35 mg, about 7 mg to about 13 mg, about 9 mg to about 11 mg, about 20 mg to about 40 mg, about 25 mg to about 35 mg, and about 29 mg to about 31 mg. In further embodiments, the amount of the alkalinizing agent is about 10 mg. In further embodiments, the amount of the alkalinizing agent is about 20 mg. In further embodiments, the amount of the alkalinizing agent is about 30 mg. In further embodiments, the amount of the alkalinizing agent is about 40 mg. In certain embodiments, the amount of the alkalinizing agent is about 0.1 to 20.0%, about 1.0% to about 20%, about 2% to about 18.0%, about 2.0 to about 6.0%, about 3.0 to about 5.0%, about 8.0 to about 16.0%, about 10.0 to about 14.0%, about 10.0 to about 20.0%, about 12.0 to about 18.0%, about 15.0% to about 17.0%, about 1.0%, about 2.0%, about 3.0%, about 4.0%, about 5.0%, about 6.0%, about 7.0%, about 8.0%, about 9.0%, about 10.0%, about 11.0%, about 12.0%, about 13.0%, about 14.0%, about 15.0 percent, about 16.0%, about 17.0%, about 18.0%, about 19.0%, and about 20.0%, by weight of the lozenge. In certain embodiments, the percent weights in the lozenges provided above is from two or more alkalinizing agents.

In another embodiment of the present invention, the alkalinizing agent included in the compressed lozenge may be aluminum carbonate, aluminum hydroxide, ammonium carbonate, ammonium solution, calcium carbonate, calcium phosphate, diethanolamine, magnesium carbonate, magnesium hydroxide, magnesium oxide, magnesium trisilicate, monoethanolamine, potassium bicarbonate, potassium carbonate, potassium citrate, potassium hydroxide, sodium acetate, sodium bicarbonate, sodium carbonate, sodium citrate, sodium hydroxide, sodium phosphate dibasic, sodium phosphate monobasic, sodium phosphate tribasic, triethanolamine, tromethane, and combinations thereof. In some embodiments, the alkalinizing agent is magnesium oxide, potassium carbonate, sodium phosphate tribasic, sodium carbonate, sodium hydroxide and combinations thereof. In other embodiments, the alkalinizing agent is sodium carbonate and/or sodium hydroxide. In another specific embodiment, the alkalinizing agent is sodium carbonate. In yet other embodiments, the alkalinizing agent is sodium hydroxide.

In another specific embodiment, the alkalinizing agent may also be a buffering agent. As stated above, the inclusion of an alkalinizing agent to the lozenge allows for an increase in the pH, thus reducing the ionization of memantine in the oral cavity, and thereby improving passive absorption of memantine in the oral mucosa. Accordingly, adding an alkalinizing agent may provide more control over the pH in the oral cavity, and thus provide more consistent absorption and consistent pharmacokinetics. In another specific embodiment, the alkalinizing agent may be a buffering agent, such as an alkaline buffering agent. Alkaline buffering agents are mixtures of weak bases and their conjugate acid(s), such as, for example, sodium carbonate/sodium bicarbonate, barbitone sodium/hydrochloric acid, trisaminomethane/hydrochloric acid, sodium tetraborate/hydrochloric acid, glycine/sodium hydroxide, sodium carbonate/sodium hydrogen carbonate, sodium tetraborate/sodium hydroxide, sodium bicarbonate/sodium hydroxide, sodium hydrogen orthophosohate/sodium hydroxide, and potassium chloride/sodium hydroxide. In another specific embodiment, the alkalinizing agent is sodium carbonate and sodium bicarbonate.

In a specific embodiment, sodium hydroxide is present in the amount of about 4 mg to about 8 mg. In another embodiment, sodium hydroxide is present in the amount of about 5 mg to about 7 mg. In another embodiment, sodium hydroxide is present in the amount of about 6 mg to about 8 mg. In another embodiment, sodium hydroxide is present in the amount of about 6 mg to about 8 mg.

In another embodiment, the alkalinizing agent present in the lozenge is sodium carbonate. In a specific embodiment, sodium carbonate is present in the amount of about 1 mg to about 35 mg. In another embodiment, sodium carbonate is present in the amount of about 3 mg to about 25 mg. In another embodiment, sodium carbonate is present in the amount of about 5 mg to about 15 mg. In another embodiment, sodium carbonate is present in the amount of about 7.5 mg to about 12 mg. In another embodiment, sodium carbonate is present in the amount of about 3 mg. In another embodiment, sodium carbonate is present in the amount of about 9 mg.

In another embodiment, the alkalinizing agent present in the lozenge is sodium bicarbonate. In a specific embodiment, sodium bicarbonate is present in the amount of about 1 mg to about 35 mg. In another embodiment, sodium carbonate is present in the amount of about 3 mg to about 25 mg. In another embodiment, sodium carbonate is present in the amount of about 5 mg to about 15 mg. In another embodiment, sodium carbonate is present in the amount of about 7 mg. In another embodiment, sodium carbonate is present in the amount of about 21 mg. In another embodiment, sodium carbonate is present in an amount of about 1 mg to about 10 mg. In another embodiment, sodium bicarbonate is present in the amount of about 5 mg to about 25 mg. In another specific embodiment, the alkalinizing agent is sodium carbonate and sodium bicarbonate, wherein sodium carbonate is present in the amount of about 1 mg to about 10 mg and sodium bicarbonate is present in the amount of about 5 mg to about 25 mg. In another specific embodiment, the alkalinizing agent is sodium carbonate and sodium bicarbonate, wherein sodium carbonate is present in the amount of about 9 mg and sodium bicarbonate is present in the amount of about 21 mg. In another specific embodiment, the alkalinizing agent is sodium carbonate and sodium bicarbonate, wherein sodium carbonate is present in the amount of about 3 mg and sodium bicarbonate is present in the amount of about 7 mg.

In another specific embodiment, the alkalinizing agent is sodium carbonate and sodium bicarbonate, wherein sodium carbonate is present in the amount of about 1 mg to about 35 mg and sodium bicarbonate is present in the amount of about 1 mg to about 35 mg. In another specific embodiment, the alkalinizing agent is sodium carbonate and sodium bicarbonate, wherein sodium carbonate is present in the amount of about 3 mg to about 25 mg and sodium bicarbonate is present in the amount of about 3 mg to about 25 mg. In another specific embodiment, the alkalinizing agent is sodium carbonate and sodium bicarbonate, wherein sodium carbonate is present in the amount of about 5 mg to about 15 mg and sodium bicarbonate is present in the amount of about 5 mg to about 15 mg. In another specific embodiment, the alkalinizing agent is sodium carbonate and sodium bicarbonate, wherein sodium carbonate is present in the amount of about 3 mg and sodium bicarbonate is present in the amount of about 7 mg. In another specific embodiment, the alkalinizing agent is sodium carbonate and sodium bicarbonate, wherein sodium carbonate is present in the amount of about 9 mg and sodium bicarbonate is present in the amount of about 21 mg.

In some embodiments, the compressed lozenge further comprises one or more coating agents. In certain embodiments, the one or more coating agents encapsulate the memantine. In other embodiments, the one or more coating agents encapsulate the alkalinizing agent. In yet other embodiments, the one or more coating agents encapsulate both the memantine and the alkalinizing agent.

In some embodiments, the compressed lozenge further comprises one or more ion exchange resins. In certain embodiments the one or more ion exchange resins are complexed with the memantine.

In some embodiments, the compressed lozenge comprises two or more layers, wherein all or substantially all of the memantine is in a first layer, and all or substantially all of the alkalinizing agent is in a second layer. In certain embodiments, the compressed lozenge is bilayer, wherein all or substantially all of the memantine is in a first layer, and all or substantially all of the alkalinizing agent is in a second layer. In other embodiments, the first layer, the second layer, or both the first layer and the second layer further comprise one or more coating agents. In yet other embodiments, the first layer comprises one or more ion exchange resins.

In another embodiment, the lozenges may be manufactured to mask the taste or adverse organoleptic properties of memantine or specific pharmaceutically acceptable excipients described above. In a specific embodiment, specific ingredients may be mixed with a masking ingredient before the addition of other ingredients. In a specific embodiment, the memantine may be masked with Pearlitol Flash. For example, memantine and Pearlitol Flash may be premixed before the addition of other pharmaceutically acceptable excipients, thus entrapping the memantine within the Pearlitol Flash pores, thereby reducing undesirable organoleptic properties such as poor taste and mouthfeel from the drug in the oral cavity. See Example 8. In a specific embodiment, the lozenges may be manufactured to minimize the contact between the alkalinizing agent and memantine when in the oral cavity. Ina specific embodiment, the alkalinizing agent may be sodium carbonate and/or sodium bicarbonate. In another embodiment, sodium carbonate and/or sodium bicarbonate may be premixed with a pharmaceutically acceptable excipient, such as Neuselin US2, thereby absorbing the carbonates to the porous surface. See Example 9. In another embodiment, memantine may be premixed with a pharmaceutically acceptable excipient, such as Neuselin US2. See Example 10. This allows for absorption of the memantine into the pores of Neuselin, and thereby minimizes direct contact with alkalinizing agents such as sodium carbonate and/or sodium bicarbonate. In another embodiment, memantine may be premixed with a pharmaceutically acceptable excipient for improving uniform dispersion of the drug in the lozenge. For example, memantine can be mixed with Neuselin or Pearlitol Flash thus absorbing the drug onto the pharmaceutically acceptable excipient. See Examples 8-10.

In another embodiment, the manufacturing process may include adding a sodium carbonate solution to a premixture substrate. Upon evaporation of the water, the sodium carbonate is uniformly distributed within the substrate resulting in a diluted sodium carbonate matrix and promoting improved organoleptic properties for the lozenges. See Examples 11-14. In another embodiment, both the sodium carbonate and sodium bicarbonate is in a solution and added to a premixture substrate. See Examples 15-16.

In another embodiment, the manufacturing may process may include a sodium carbonate/sodium bicarbonate/binder solution. This solution may be adsorbed onto the surface of a substrate such as a Pearlitol Flash and Avicel substrate. Upon evaporation of the water, the sodium carbonate/sodium bicarbonate is uniformly distributed and strongly bonded to the substrate resulting in stronger granules which may also provide a diluted sodium carbonate/sodium bicarbonate within the MMT lozenges matrix and improved organoleptic properties for the lozenges. In another embodiment, the lozenges may be prepared by wet granulation. In a specific embodiment, the lozenges may comprise two different granules, thereby minimizing the direct contact of individual particles of memantine and sodium carbonate/sodium bicarbonate, thereby resulting in improvement of the organoleptic of the lozenge when in the mouth.

In some embodiments, the compressed lozenge exhibits a disintegration time of about 30 seconds to about 5 minutes. In another embodiment, the compressed lozenge exhibits a disintegration time of about 1 minute to about 5 minutes. In another embodiment, the compressed lozenge exhibits a disintegration time of about 1 minute to about 4 minutes. In another embodiment, the compressed lozenge exhibits a disintegration time of about 1.5 minutes to about 4.5 minutes. In another embodiment, the compressed lozenge exhibits a disintegration time of about 2 minute to about 3 minutes.

In some embodiments, the dissolution of the compressed lozenges may be within a period of about 1, 2, 3, 4, 5, 6, 7, 8, 9, 10, 11, 12, 13, 14, 15, 20, 25, 30, 35, 40, 45, 50, 55, or 60 minutes. In a specific embodiment, the dissolution of the compressed lozenge may occur within about 10 minutes. In a specific embodiment, the dissolution of the compressed lozenge may occur within about 15 minutes. In another embodiment, the dissolution periods listed above may occur using a modified USP method (50 rpm paddle speed).

In some embodiments, the compressed lozenge exhibits a dissolution time (time to 100% memantine release, using modified USP Method, with 50 rpm paddle speed) of about 5 minutes to about 15 minutes (FIG. 8). In another embodiment, the compressed lozenge exhibits a dissolution time of about 5 minute to about 10 minutes.

In another embodiment, the dissolution may occur when in the oral cavity of a subject.

### Methods of Preparing Lozenges

Another aspect of the invention relates to a method of making a lozenge comprising combining memantine with an alkalinizing agent.

In some embodiments, the method further comprises combining the memantine and the alkalinizing agent with one or more pharmaceutically acceptable excipients. In certain embodiments, the one or more pharmaceutically acceptable excipients are selected from selected from fillers, binders, diluents, sweetening agents, disintegrants, moisture scavengers, colorants, flavorants, permeation enhancers, solvents and co-solvents.

In some embodiments, the alkalinizing agent is sodium hydroxide or sodium carbonate. In further embodiments, the alkalinizing agent is sodium hydroxide. In some embodiments, the alkalinizing agent is sodium carbonate. In some embodiments, the alkalinizing agent is sodium bicarbonate. In some embodiments, the alkalinizing agent is sodium carbonate and sodium bicarbonate.

In some embodiments, the method further comprises coating the memantine with a coating agent. In further embodiments, the method further comprises further comprises coating the alkalinizing agent with a coating agent. In other embodiments, the method further comprises coating the memantine and the alkalinizing agent with a coating agent.

In some embodiments, the method further comprises complexing the memantine with one or more ion exchange resins.

In some embodiments, the method further comprises packing the lozenge in such a manner so as to protect the lozenge from damage, moisture and/or oxidation. In certain embodiments, the method further comprises packing the lozenge in a blister pack or bottle. In certain embodiments, the method further comprises packing the lozenge in a high density polyethylene (HDPE) bottle and, optionally, with a desiccant.

In some embodiments, the method further comprises pre-mixing memantine and the alkalinizing agents in a granulating solution followed by high shear and/or fluid bed granulation of granulating solution with other dry powder excipients.

### Solution Formulation

In some embodiments, the memantine formulation may be in the dosage form of a solution. In a specific embodiment, the memantine concentration in the solution may be about 5 mg/mL to about 20 mg/mL. In another embodiment, the memantine concentration in the solution may be about 8 mg/mL to about 16 mg/mL. In another embodiment, the memantine concentration in the solution may be about 10 mg/mL to about 14 mg/mL. In another embodiment, the memantine concentration in the solution may be about 12 mg/mL.

In another embodiment, the solution may include one or more alkalinizing agents or buffering agents. In a specific embodiment the alkalinizing agent may be sodium bicarbonate and sodium carbonate. In a specific embodiment, the sodium bicarbonate and sodium carbonate ratio may be about 15:1 to about 1:15. In another specific embodiment, the sodium bicarbonate and sodium carbonate ratio may be about 10:1 to about 8:1. In another specific embodiment, the sodium bicarbonate and sodium carbonate ratio may be about 9:1. In another embodiment of the present invention, the one or more alkalinizing agents or buffering agents may be provided in a concentration of about 0.01M to about 0.5M. In another embodiment of the present invention, the one or more alkalinizing agents or buffering agents may be provided in a concentration of about 0.05M to about 0.2M. In another embodiment of the present invention, the one or more alkalinizing agents or buffering agents may be provided in a concentration of about 0.1 M. In another specific embodiment, the sodium bicarbonate and sodium carbonate ratio may be about 9:1 at a concentration of about 0.1M. See, e.g., Example 22.

### Molding Methods

In some embodiments, the lozenge is a candy lozenge. In particular embodiments, the method further comprises combining the memantine and the alkalinizing agent with one or more sweetening agents. In certain embodiments, the method further comprises heating the memantine, the alkalinizing agent and the one or more sweetening agents at a sufficiently high temperature for a sufficient amount of time to evaporate substantially all moisture. In particular embodiments, the resulting lozenge has a moisture content of about 0.5% w/w to 10%. In other embodiments, the lozenge has a moisture content of about 0.5 to 6.0% w/w. In other embodiments, the lozenge has a moisture content of about 0.5 to 4.0% w/w. In other embodiments, the lozenge has a moisture content of about 0.5 to 3.0% w/w. In other embodiments, the lozenge has a moisture content of about 0.5 to 2.0% w/w. In yet other embodiments, the lozenge has a moisture content of about 0.5, about 1.0, about 1.5%, about 2.0%, about 2.5%, about 3.0%, about 3.5%, about 4.0%, about 4.5%, about 5.0% w/w, about 5.5% w/w, about 6% w/w, about 6.5% w/w, about 7.0% w/w, about 7.5% w/w, about 8.0% w/w, about 8.5% w/w, about 9.0% w/w, about 9.5% w/w, or about 10.0% w/w, inclusive of all values and ranges therebetween.

In some embodiments, the lozenge is a candy lozenge comprising two or more layers, wherein all or substantially all of the memantine is in a first layer, and all or substantially all of the alkalinizing agent is in a second layer. In certain embodiments, the candy lozenge is bilayer, wherein all or substantially all of the memantine is in a first layer, and all or substantially all of the alkalinizing agent is in a second layer.

In some embodiments, the method further comprises preparing the first layer by combining the memantine with one or more pharmaceutically acceptable excipients independently selected from diluents, sweetening agents and colorants. In certain embodiments, the one or more pharmaceutically acceptable excipients comprise one or more sweetening agents and the method further comprises heating the combination of memantine and one or more pharmaceutically acceptable excipients to a sufficiently high temperature so as to allow the one or more sweetening agents to dissolve. In further embodiments, the method further comprises heating the combination to a temperature of about 165° C., and cooling the combination to a temperature of about 130 to 140° C.

In additional embodiments, the one or more sweetening agents comprise isomalt and acesulfame potassium.

In some embodiments, the method further comprises, after the cooling step, adding to the combination one or more pharmaceutically acceptable excipients independently selected from flavorants, permeation enhancers, solvents and co-solvents. In certain embodiments, the one or more pharmaceutically acceptable excipients comprise menthol.

In some embodiments, the method further comprises the steps of preparing the second layer, which steps comprise: combining one or more sweetening agents with water; and heating the combination of water and one or more sweetening agents to a sufficiently high temperature so as to allow the one or more sweetening agents to dissolve.

In some embodiments, the method further comprises: heating the combination to a temperature of about 165° C.; and cooling the combination to a temperature of about 130 to 140° C. In certain embodiments, the one or more sweetening agents comprise isomalt.

In some embodiments, the method further comprises, after the cooling step, adding the alkalinizing agent and one or more reducing agents to the combination. In certain embodiments, the one or more reducing agents comprise sodium metabisulfite (SMBS).

In some embodiments, the method further comprises forming the first layer and the second layer with a candy depositor. The candy depositor may be a single depositor or a double depositor. In certain embodiments, the candy depositor is a single depositor. In other embodiments, the candy depositor is a double depositor. In other embodiments, the method comprises forming the first and second layers with equivalents of candy depositors suitable for high-speed, high-volume manufacturing of multilayer candy lozenges.

In some embodiments, the method further comprises combining the first layer and the second layer to form a bilayer candy lozenge.

In some embodiments, the method further comprises coating the memantine with a coating agent. In further embodiments, the method further comprises further comprises coating the alkalinizing agent with a coating agent. In other embodiments, the method further comprises coating the memantine and the alkalinizing agent with a coating agent.

In some embodiments, the method further comprises complexing the memantine with one or more ion exchange resins.

### Compression Methods

In some embodiments, the lozenge is a compressed lozenge. In particular embodiments, the method further comprises, before combining the memantine with the alkalinizing agent: granulating the memantine to form granulated memantine; and granulating the alkalinizing agent to form granulated alkalinizing agent.

In some embodiments, the method further comprises milling the memantine. In some embodiments, the method further comprises milling the memantine before granulating or dry blending the memantine. In a specific embodiment, the mill may be a Comill conical mill that is fitted with 18R screen and round impeller. In some embodiments, the lozenges may be directly compressed.

In some embodiments, the method further comprises coating the granulated memantine with a coating agent. In further embodiments, the method further comprises further comprises coating the granulated alkalinizing agent with a coating agent. In other embodiments, the method further comprises coating the granulated memantine and the granulated alkalinizing agent with a coating agent.

In some embodiments, the method further comprises complexing the granulated memantine with one or more ion exchange resins.

In some embodiments, the method further comprises coating the granulated memantine with a coating agent. In further embodiments, the method comprises coating the granulated alkalinizing agent with a coating agent. In other embodiments, the method comprises coating the granulated memantine and the granulated alkalinizing agent with a coating agent.

In some embodiments, the granulating of the memantine involves wet granulation. In further embodiments, the granulating of the memantine involves wet granulation and wet milling. In other embodiments, the granulating of the memantine comprises: wet granulating the memantine to form wet granulation; wet milling the wet granulation to form wet milled granulation; drying the wet milled granulation to form dried granulation; dry milling the dried granulation to form dry milled granulation; and blending the dry milled granulation.

In some embodiments, the granulating of the alkalinizing agent involves wet granulation. In further embodiments, the granulating of the alkalinizing agent involves wet granulation and wet milling. In other embodiments, the granulating of the alkalinizing agent comprises: wet granulating the alkalinizing agent to form wet granulation; wet milling the wet granulation to form wet milled granulation; drying the wet milled granulation to form dried granulation; dry milling the dried granulation to form dry milled granulation; and blending the dry milled granulation.

In some embodiments, the wet granulating step comprises wet granulating the memantine or the alkalinizing agent with one or more pharmaceutically acceptable excipients independently selected from sweetening agents, colorants, fillers and binders. In other embodiments, the wet granulating step comprises wet granulating the memantine or the alkalinizing agent with isomalt, microcrystalline cellulose and povidone. In some embodiments, the method further comprises blending the granulated memantine and the granulated alkalinizing agent with one or more pharmaceutically acceptable excipients. In certain embodiments, the one or more pharmaceutically acceptable excipients are independently selected from sweetening agents, colorants, flavorants, permeation enhancers, solvents, co-solvents, fillers, binders, disintegrants, lubricants, glidants and moisture scavengers. In further embodiments, the one or more pharmaceutically acceptable excipients comprise isomalt, acesulfame potassium, menthol, magnesium aluminometasilicate, magnesium stearate, polyethylene glycol 8000 and sodium stearyl fumarate. In still other embodiments, the one or more pharmaceutically acceptable excipients comprise isomalt, acesulfame potassium, menthol, magnesium aluminometasilicate, polyethylene glycol 8000 and magnesium stearate. In a specific embodiment, the one or more pharmaceutically acceptable excipients are independently selected from the group consisting of a binder, a sugar or sugar substitutes, a filler, a disintegrant, a lubricant, a moisture scavenger and combinations thereof.

In another specific embodiment, the one or more pharmaceutically acceptable excipients are independently selected from the group consisting of a microcrystalline cellulose, magnesium stearate, starch, mannitol, sucralose, magnesium aluminometasilicate and combinations thereof.

In some embodiments, the method further comprises compressing the memantine, the alkalinizing agent and the one or more pharmaceutically acceptable excipients to form a compressed lozenge. In certain embodiments, the memantine, the alkalinizing agent and the one or more pharmaceutically acceptable excipients are compressed in a tablet die.

In some embodiments, the method further comprises sampling the granulated memantine for potency before the compressing step.

In some embodiments, the lozenge is a compressed lozenge comprising two or more layers, wherein all or substantially all of the memantine is in a first layer, and all or substantially all of the alkalinizing agent is in a second layer. In certain embodiments, the compressed lozenge is bilayer, wherein all or substantially all of the memantine is in a first layer, and all or substantially all of the alkalinizing agent is in a second layer.

### Methods of Treating Cough

Another aspect of the invention relates to a method of treating cough, comprising administering to a patient in need thereof a lozenge selected from any of the lozenges, including specific embodiments and combinations of embodiments, described herein.

For any of the methods described herein, the cough may be acute, subacute or chronic. In some embodiments, the cough is acute. In other embodiments, the cough is subacute. In yet other embodiments, the cough is chronic.

In some embodiments, the patient is human. In certain embodiments, the patient is a pediatric patient of about 18 years of age or younger. In additional embodiments, the patient is a pediatric patient of about 2 to 18 years of age, inclusive of all ranges and subranges therebetween. In particular embodiments, the patient is a pediatric patient of about 6 to 18 years of age. In other embodiments, the patient is a pediatric patient of about 6 to 12 years of age. In yet other embodiments, the patient is a pediatric patient of about 2 to 5 years of age. In further embodiments, the patient is a geriatric patient of about 65 years of age or older.

For any of the methods described herein, the lozenge, compound or pharmaceutical composition may be administered one, two, three, four or five or more times a day. In some embodiments, it is administered one to four times a day. In other embodiments, it is administered one to two times a day. In yet other embodiments, it is administered once a day. In further embodiments, it is administered two times a day. In yet another embodiments, it is administered three times a day.

Suitable doses of the lozenge, compound or pharmaceutical composition described herein may depend in part on the characteristics of the patient (e.g., age, weight, gender) and the type or severity of the cough being treated. In some embodiments, the patient is a human over about 12 years of age and the lozenge, compound or pharmaceutical composition is administered in about one dose at least once a day, at least twice a day, once a day, or twice a day. In other embodiments, the patient is a human from about 6 to about 12 years of age, and the lozenge, compound or pharmaceutical composition is administered in about ½ dose (relative to patients over about 12 years of age) once a day or twice a day. In another embodiment, the patient is a human from about 2 to about 6 years of age, and the lozenge, compound or pharmaceutical composition is administered in an about ¼ dose (relative to patients over about 12 years of age) once a day or twice a day.

The specific embodiments of the invention may be directed to one, some or all of the above-indicated aspects, and the particular aspects of the invention may encompass one, some or all of the above- and below-indicated embodiments, as well as other embodiments. The following examples are illustrative of the present invention and are not intended to be limitations thereon.

### Example 1

### Pharmacokinetic Evaluation of Memantine After Buccal Administration in Male Beagle Dogs

The pharmacokinetics of memantine were evaluated after buccal administration in male beagle dogs. Memantine was formulated in water, 3.3 mg/mL sodium hydroxide in water, or 7.5 mg/mL sodium carbonate in water. All dogs received a 0.4 mg/kg dose of memantine. Plasma levels of memantine were determined by LC-MS/MS. Pharmacokinetic parameters were determined for the memantine plasma data.

For dosing, dogs were anesthetized with an IV injection of ketamine/diazepam, and maintained by isoflurane intubation during the buccal administration. The dosing solution was pipetted into a circular cylinder to concentrate the dosing solution on one area of the mucosa. At just prior to the 15 minute sample time point, the oral cavity was rinsed with 5 mL of water and dried with gauze. Immediately after the rinse, the 15 minute sample was collected.

Table 1 provides a summary of pharmacokinetic findings, comparing oral and buccal dosing routes for memantine compositions containing a urinary acidification agent (oral route) or a buffering agent (buccal route) to increase local pH. As shown in Table 1, urinary acidification increases the rate of elimination as shown by the reduced T_{1/2} values relative to controls, and buccal administration increases the rate of absorption (as shown by decreased Tₘₐₓ and increased Cₘₐₓ values), particularly when alkalinizing agents are used to increase the local pH of the buccal environment.

TABLE 1
Summary of PK Findings

| | | | | | | | |
|---|---|---|---|---|---|---|---|
| MMT Dose | 0.4 mg/kg | 0.4 mg/kg | 0.4 mg/kg | 0.4 mg/kg | 0.4 mg/kg | 0.4 mg/kg | 0.4 mg/kg |
| Route | Oral | Oral | Oral | Oral | Buccal | Buccal | Buccal |
| Objective | | ↓ Urine | ↓ Urine | ↓ Urine | | ↑ Buccal | ↑ Buccal |
| | | pH | pH | pH | | pH | pH |
| Concomitant | Control | 15 mg/kg | 25 mg/kg | 30 mg/kg | Control | 3.3 mg/ml | 7.5 mg/ml |
| agent | Ave | NH₄Cl² | NH₄Cl | NH₄Cl | | NaOH | Na₂CO₃ |
| Cₘₐₓ | 22 | 25 | 20 | 25 | 23 | 52 | 54 |
| (ng/mL) | | | | | | | |
| Cₘₐₓ/Dose | 5.2 | 6.8 | 5.2 | 5.4 | 6.7 | 12.9 | |
| Tₘₐₓ (h) | 1.7 | 2.0 | 1.5 | 1.1 | 0.75 | 0.25 | 0.45 |
| | | | | | | P < 0.05 | |
| | | | | | | vs. C | |
| AUC₀₋ₜ | 190 | 233 | 169 | 163 | 119 | 108 | 179 |
| (ng · h/mL) | | | | | | | |
| AUC_{0-∞} | 210 | 256 | 179 | 168 | 125 | 112 | 184 |
| (ng · h/mL) | | | | | | | |
| T_{1/2} (h) | 6.5 | 6.7 | 5.5 | 4.8 | 5.2 | 5.2 | 4.5 |
| | | | | P < 0.05 | | | |
| | | | | vs. C | | | |

### Example 2

### Pharmacokinetic Evaluation of Memantine after Buccal Administration in Male Beagle Dogs

The pharmacokinetics of memantine were evaluated after buccal administration in male beagle dogs using procedures similar to those used in Example 1, except that menthol or menthol and ammonium chloride where co-administered with the sodium hydroxide (Table 2). Table 2 provides a summary of pharmacokinetic findings, comparing memantine compositions containing an alkalinizing agent to increase local pH and a permeation enhancer (menthol), and optionally a urinary acidifying agent. As shown in Table 2, the combination of an alkalinizing agent and permeation enhancer, and substantially increases the Cₘₐₓ/Dose and substantially decreases Tₘₐₓ, and significantly reduces T_{1/2} compared to the control. Further addition of a urinary acidifying agent (e.g., NH₄Cl) further reduces T_{1/2}, indicating more rapid elimination of memantine.

TABLE 2
Summary of PK Findings

| | | | | |
|---|---|---|---|---|
| MMT Dose | 0.4 mg/kg | 0.4 mg/kg | 0.4 mg/kg | 0.4 mg/kg |
| Route | Oral | Oral | Oral | Oral |
| Objective | | ↑ Buccal pH | ↑ Buccal pH | ↑ Buccal pH |
| | | | ↓ Urine pH | ↓ Urine pH |
| Concomitant | Control | 3.3 mg/ml | 3.3 mg/ml | 3.3 mg/ml |
| agent | | sodium | sodium | sodium |
| | | hydroxide + | hydroxide + | hydroxide + |
| | | 5 mg/ml | 5 mg/ml | 5 mg/ml |
| | | menthol | menthol + | menthol + |
| | | | 30 mg/kg | 20 mg/kg |
| | | | NH₄Cl | NH₄Cl |
| Cₘₐₓ (ng/mL) | 34.5 | 77.0 | 43.0 | 126 |
| Cₘₐₓ/Dose | 7.91 | 18.16 | 9.18 | 26.03 |
| Tₘₐₓ (h) | 0.75 | 0.20 | 0.25 | 0.15 |
| | | P < 0.05 | P < 0.05 | P < 0.05 |
| | | vs. C | vs. C | vs. C |
| AUC₀₋ₜ | 120 | 113 | 103 | 134 |
| (ng · h/mL) | | | | |
| AUC_{0-∞} | 132 | 126 | 125 | 143 |
| (ng · h/mL) | | | | |
| T_{1/2} (h) | 7.18 | 5.05 | 4.33 | 4.60 |
| | | P < 0.05 | P < 0.05 | P < 0.05 |
| | | vs. C | vs. C | vs. C |

### Example 3

### Preparation of Candy Lozenges

Bilayer candy lozenges are prepared according to the constituents in Table 3 and Table 4.

TABLE 3
Constituents in First Layer of Bilayer Candy Lozenge

| Constituent | mg/lozenge | % w/w | g/batch |
|---|---|---|---|
| Galen IQ 990 (Isomalt) | 2425.2 | 78.07 | 1600.63 |
| Deionized Water | | 19.52 | 400.16 |
| Memantine HCl | 7.5 | 0.24 | 4.95 |
| Acesulfame Potassium | 12 | 0.39 | 7.92 |
| Red Dye | 20 | 0.64 | 13.2 |
| Blue Dye | 0.3 | 0.01 | 0.2 |
| Mineral Oil | 15 | 0.48 | 9.9 |
| Black Cherry FALT098 | 15 | 0.48 | 9.9 |
| Menthol Crystals | 5 | 0.16 | 3.3 |
| Total | 2500 | 100 | 2050.16 |

### Steps for Preparing First Layer

1. Combine deionized water, isomalt, red and blue dyes, acesulfame potassium and memantine.
2. Heat slowly to >90° C. to allow isomalt to fully dissolve.
3. Increase heat to 165° C.
4. Cool to 130-140° C. and maintain temperature.
5. Add mineral oil, menthol crystals, and black cherry flavor.
6. Form lozenges with depositor.

TABLE 4
Constituents in Second Layer of Bilayer Candy Lozenge

| Constituent | mg/lozenge | % w/w | g/batch |
|---|---|---|---|
| Galen IQ 990 | 1985.3 | 79.21 | 992.7 |
| Deionized Water | QS | 19.8 | 248.2 |
| Sodium Hydroxide, 10N | 6.7 | 0.67 | 8.4 |
| Sodium Metabisulfite | 8 | 0.32 | 4.0 |
| Total | 2000 | 100 | 1253.2 |

### Steps for Preparing Second Layer

1. Combine water and isomalt.
2. Heat slowly to >90° C. to allow isomalt to fully dissolve.
3. Increase heat to 165° C.
4. Cool to 130-140° C. and maintain temperature.
5. Add SMBS and sodium hydroxide.
6. Observe color changes.
7. Form lozenges with depositor.

### Example 4

### Preparation of Compressed Lozenges

Compressed lozenges are prepared according to the following process steps.

### Step 1: Preparation of Alkalinizing Agent Granulation

1. Dispensing. For each sublot, dispense the following materials: Avicel PH 101; Galen IQ 810; Plasdone K-29/32; FD&C Red 40 LDL; FD&C Blue 2 LDL, 10N NaOH and Deionized Water.
2. Wet Granulation. For each sub-lot, add Avicel PH 101 to the high shear granulator bowl. Add NaOH solution to the granulation, mixing for a total of 9 minutes. Stop the granulator, scrape down sides and bottom of the bowl. Pass Galen IQ 810 through a 20 mesh screen. Add a portion of the screened material to the poly bags containing FD&C Red 40 LDL and FD&C Blue 2 LDL and bag-blend. Add FD&C Red 40 LDL, FD&C Blue 2 LDL, Galen IQ 810, and Plasdone K-29/32 to the granulator bowl. Continue mixing for a total of 2 minutes. Stop the granulator, scrape down sides and bottom of the bowl. Discharge sublot alkalinizing agent granulation into a poly bag.
3. Sub-Batches. Repeat the steps for additional sublots. Discharge sublots into separate poly bags.
4. Wet Milling Drying Dry Milling & Blending. Pass all wet granulation sublots through a Comil conical mill. Dry the granulation in a convection tray drying oven set to 40°-50° C. until the moisture content is NMT 5.0% LOD. Discharge the dried granulation into a poly bag. Pass all dried granulation through the Comil conical mill. Collect the milled granulation in a poly bag. Collect all granulation waste. Add the milled granulation to the v-blender and blend for approximately 5 minutes. Reconcile weights of materials.
5. Powder Characterization. Measure the flow of the dried granulation with a Flodex apparatus, Test the bulk/tapped density of the dried granulation. Test the particle size distribution of the dried granulation.

### Step 2: Preparation of Memantine HCl Granulation

1. API Milling. Mill memantine HCl. Dispense memantine HCl and pass it through a Comil conical mill. Collect the milled memantine HCl in a poly bag. Collect all milling waste.
2. Dispensing. For each sublot, dispense the following components: Avicel PH 101, Galen IQ 810, memantine HCl (milled), Plasdone K-29/32, and deionized water. Dissolve Plasdone K-29/32 in deionized water.
3. Wet Granulation. For each sublot, pass Galen IQ 810 through a 20 mesh screen and add in high shear granulator bowl, followed by memantine HCl. Add a portion of Avicel to the memantine poly bag and bag blend to remove any remaining memantine HCL and add to the granulator bowl. Add Avicel. Premix the raw materials for 2 minutes. Add Plasdone Solution to the granulation and mix for a total of 11 minutes. Stop the granulator, and scrape down the sides and bottom of the bowl. Discharge granulation into a poly bag.
4. Wet Milling Drying Dry Milling & Blending. Pass wet granulation through the Comil conical mill. Dry the granulation in a convection try drying oven set to 40°-50° C. until the moisture content is NMT 5.0% LOD. Discharge the dried granulation into a poly bag. Pass all dried granulation through the Comil conical mill. Collect the milled granulation in a poly bag. Collect all granulation waste. Add the milled granulation to the v-blender and blend for approximately 5 minutes. Collect samples for testing. Collect the completed granulation in a poly bag. Reconcile weights.
5. Powder Characterization. Measure the flow of the dried granulation using the Flodex apparatus. Test the bulk/tapped density of the dried granulation. Test the particle size distribution of the dried granulation.

### Step 3: Preparation of Compressed Lozenges

The following process description applies to 3 mg, 6 mg, 9 mg and 12 mg lozenge batches. The strength is achieved by adjusting amount of MMT granulation that is added. The amount of added isomalt is adjusted so that the weight and quantities of all other ingredients remains the same across strengths.
1. Dispensing. Dispense the following components: Galen IQ 720; Black Chemy FALU906; Menthol 3433-002; Neusillin US2; Acesulfame K; Polyglykol 8000 PF; PRUV; alkalinizing agent Granulation; and Memantine HCl granulation.
2. Blending. Add Alkalinizing Agent Granulation, Memantine HCl Granulation to the v-blender. Pass Black Chemy FALU906, Menthol 3433-002, Neusilin US2, Acesulfame K, Polyglykol 8000 PF, and PRUV through a 20 mesh screen. Add them to the v-blender followed by Galen IQ 720. Blend for 10 minutes. Discharge blend into a poly bag. Reconcile weights.
3. Tableting. Charge the blend into the hopper. Adjust the die fill amount and compression parameters to yield a tablet with the target weight and hardness. Collect all finished tablets in a poly bag. Collect waste in a poly bag. Reconcile weights.
4. Tablet Characterization. Evaluate the weight, hardness, and thickness of 10 tablets. Evaluate the friability; measure the pH, measure the disintegration time.
5. Packaging. Package into 75 cc HDPE bottles containing 1 gram molecular sieve desiccant and capped with an induction sealed 38 mm CRC cap. Fill bottle with 24 tablets. Add 3 1 g molecular sieve desiccant in each bottle.

### Example 5

### Stability of Memantine in Alkaline Conditions

The stability of memantine is studied within a pH range in both solution and in a lozenge dosage form. It is determined that when memantine is included in a buffer solution at a pH 8.0 or higher, memantine degrades and/or precipitates out of solution. It is determined that in a solution with a pH of 8.0, only about 85.3 percent of memantine is recovered; in a solution with a pH of 9.0, only about 29.8 percent of memantine is recovered; and in a solution with a pH of 10.0, only about 0.7 percent of memantine is recovered. In a solution with a pH of 1.0, it is determined that 97.6 percent of memantine is recovered and thus memantine is determined to be stable. It is also determined that memantine is unstable in a lozenge dosage form when it is in contact with an alkalinizing agent. It is determined that when memantine and sodium hydroxide are added together in a lozenge dosage form, only about 40% to about 60% memantine is recovered, with the remaining memantine degrading and/or precipitating out during the lozenge preparation process. It is determined that when memantine and sodium carbonate are added together in a lozenge dosage form, only about 40% to about 60% memantine is recovered, with the remaining memantine degrading and/or precipitating out during the lozenge preparation process.

### Example 6

### Preparation of Compressed Lozenges

Compressed lozenges with alkalinizing agents sodium bicarbonate and sodium carbonate may also be prepared according to the following process steps:
1. Dispensing. The following ingredients are then dispensed in a pre-tared poly bag: memantine HCl (milled), Pearlitol Flash; flavor (such as black cherry), Menthol; Magnesium Alumino Metasilicate (Neusillin), Sucralose, Sodium Bicarbonate, and Sodium Carbonate Anhydrous, Avicel PH 101
2. Milling. Memantine HCl is then milled at 3000 rpm through a Comill conical mill that is fitted with 18R screen and round impeller.
3. Blending. The following ingredients are then de-lumped through a 20 mesh screen and added to a 4 quart v-blender in this order: Pearlitol Flash, followed by Memantine HCl (milled), flavorant, Menthol, Magnesium Alumino Metasilicate, Sucralose, Sodium Bicarbonate, and Sodium Carbonate Anhydrous. Avicel PH 101 is de-lumped and added to the v-blender last. These ingredients are then blended for 10 minutes. Magnesium Stearate is de-lumped through a 20 mesh screen, added to the v-blender as a final step, and mixed for an additional 1.5 minutes with the remaining ingredients of the blend.
4. Tableting. The blend is discharged in a poly bag and compressed using a tablet press that is fitted with 5/16″ flat face beveled edge concave tooling (5 stations) and gravity feeder.
5. Tablet characterization. The die fill and compression parameters are adjusted to yield a tablet target weight of 250 mg, hardness of 2-3 kp, disintegration time in water of NMT 5 minutes, and friability of NMT 1%.
6. Packaging. The tablets are then packaged in 30 cc round HDPE bottles or cold form (foil foil) blister strips. A 1 g molecular sieve desiccant in each bottle is inserted and the bottle is closed and induction sealed.

TABLE 5
Example of ingredients in a compressed 250 mg Lozenge with
varying ranges of sodium bicarbonate and sodium carbonate.

| | Lozenge with 3 mg | | Lozenge with 9 mg | |
|---|---|---|---|---|
| | of sodium carbonate, 7 | | of sodium carbonate, 21 | |
| | mg sodium bicarbonate | | mg sodium bicarbonate | |
| Constituent | mg/lozenge | % w/w | mg/lozenge | % w/w |
| Memantine HCl | 6.04 | 2.42 | 6.04 | 2.42 |
| Pearlitol Flash | 204.5 | 81.78 | 183.71 | 73.48 |
| Sodium bicarbonate | 7.0 | 2.8 | 21.0 | 8.4 |
| Sodium carbonate | 3.0 | 1.2 | 9.0 | 3.6 |
| anhydrous | | | | |
| Avicel PH 101 | 12.5 | 5.0 | 12.5 | 5.0 |
| Menthol 3433-002 | 6.25 | 2.5 | 6.25 | 2.5 |
| (20%) | | | | |
| Neuselin US2 | 5.0 | 2.0 | 5.0 | 2.0 |
| Sucralose | 1.25 | 0.5 | 2.0 | 0.8 |
| Magnesium Stearate | 4.5 | 1.8 | 4.5 | 1.8 |
| Total | 250.0 | 100 | 250.0 | 100 |

### Example 7

### Preparation of Compressed Lozenges

Compressed lozenges with alkalinizing agents sodium bicarbonate and sodium carbonate may also be prepared by high shear or fluid bed granulation according to the following process steps:
1. Weigh the required quantities of sodium carbonate, sodium bicarbonate, memantine, Avicel and screen them through 30 mesh hand screen separately.
2. Weigh the required quantity of Pearlitol Flash and divide in to 2 halves.
3. Screen the materials from step 2 through 20 mesh screen.
4. Premix all the materials from step 1 and approximately half of screened Pearlitol from in a Turbula mixer for 4 minutes.
5. Granulate the premix from step 4 with 15 mL of water in a suitable container.
6. Perform moisture analysis on the wet mass and dry in a tray dryer (50-60° C.) until the LOD is in between 3-4%.
7. Weigh the required quantities Neusilin US2, sucralose, and menthol and screen them through 20 mesh hand screen.
8. Blend the granules from step 6 with screened materials from step 7 and remaining half of Pearlitol from step 2 in a Turbula mixer for 8 minutes.
9. Weigh the required quantities of magnesium stearate and screen through 30 mesh hand screen
10. Add screened magnesium stearate from step 9 to blend from step 8 and mix 90 seconds
11. Compress the blend from step 10 using appropriate tools to target weight of 250 mg and target hardness of 2-5 kP.

### Example 8

### Direct Compression—Masking MMT with Pearlitol Flash

| Component | mg/tablet | % w/w | g/batch |
|---|---|---|---|
| Memantine HCl | 18.12 | 7.25 | 7.25 |
| Pearlitol Flash | 172.38 | 68.95 | 68.95 |
| Sodium bicarbonate | 21.00 | 8.4 | 8.4 |
| Sodium carbonate | 9.00 | 3.6 | 3.6 |
| anhydrous | | | |
| Menthol 3433-002 | 6.25 | 2.50 | 2.50 |
| Neusilin US2 | 5.00 | 2.00 | 2.00 |
| Sucralose | 1.25 | 0.50 | 0.50 |
| Avicel PH 101 | 12.50 | 5.00 | 5.00 |
| Magnesium stearate | 4.50 | 1.80 | 1.80 |
| Total | 250.00 | 100.00 | 100.00 |

### Manufacturing Procedure:

1. Weigh the required quantities of MMT and Pearlitol Flash
2. Premix MMT with approx. half of the quantity of Pearlitol Flash in a suitable poly bag and screen though 30 mesh
3. Mix the blend from step 2 in a suitable poly bag for 3-5 minutes
4. Weigh the required quantities of Menthol, Neusilin US2, Sucralose and Avicel PH 101 and premix in a separate polybag and screen through 20 mesh hand screen
5. Combine blends from step 3 and 4 in a poly bag and mix for mix for 3-5 minutes
6. Weigh the required quantities of sodium carbonate and bicarbonate and pass through 20 mesh hand screen
7. Mix the screened materials from step 6 with remaining quantity of Pearlitol Flash in a poly bag for 3-5 minutes
8. Add the materials from step 7 to bag from step 5 and mix for 3-5 minutes
9. Weigh the required quantities of Magnesium stearate and screen through 30 mesh hand screen
10. Add magnesium stearate from step 8 to bag from step 7 and blend for 1-2 minutes
11. Compress the blend from step 9 into 5-10 tablets on a carver press using appropriate tools to target weight of 250 mg.

### Example 9

### Direct Compression—Masking Carbonates with Neusilin US2

| Component | mg/tablet | % w/w | g/batch |
|---|---|---|---|
| Memantine HCl | 18.12 | 7.25 | 7.25 |
| Pearlitol Flash | 162.38 | 64.95 | 64.95 |
| Sodium bicarbonate | 21.00 | 8.40 | 8.40 |
| Sodium carbonate | 9.00 | 3.60 | 3.60 |
| anhydrous | | | |
| Menthol 3433-002 | 6.25 | 2.50 | 2.50 |
| Neusilin US2 | 15.00 | 6.00 | 6.00 |
| Sucralose | 1.25 | 0.50 | 0.50 |
| Avicel PH 101 | 12.50 | 5.00 | 5.00 |
| Magnesium stearate | 4.50 | 1.80 | 1.80 |
| Total | 250.00 | 100.00 | 100.00 |

### Manufacturing Procedure:

1. Weigh the required quantities of sodium carbonate, sodium bicarbonate and Neusilin US2
2. Premix all the material from step 1 in a suitable polybag and screen though 30 mesh hand screen
3. Mix the blend from step 2 in a suitable poly bag for 3-5 minutes
4. Weight the required quantities of Pearlitol Flash and MMT
5. Premix the MMT with half the quantity of Pearlitol Flash and screen though 30 mesh hand screen
6. Mix the blend from step 5 in a suitable polybag for 3-5 minutes
7. Weigh the required quantities of menthol, sucrose and Avicel PH 101 and screen them though 20 mesh hand screen
8. Add the blends from steps 6, 7 and 3 in this order to a separate poly bag, add the remaining Pearlitol Flash and mix for 3-5 minutes
9. Weigh the required quantity of magnesium stearate and screen through 30 mesh hand screen
10. Add magnesium stearate from step 9 to bag from step 8 and blend for 1-2 minutes
11. Compress the blend from step 10 into 5-10 tablets on a carver press using appropriate tools to target weight of 250 mg.

### Example 10

### Direct Compression—Masking MMT with Neusilin US2

| Component | mg/tablet | % w/w | g/batch |
|---|---|---|---|
| Memantine HCl | 18.12 | 7.25 | 7.25 |
| Pearlitol Flash | 162.38 | 64.95 | 64.95 |
| Sodium bicarbonate | 21.00 | 8.40 | 8.40 |
| Sodium carbonate | 9.00 | 3.60 | 3.60 |
| anhydrous | | | |
| Menthol 3433-002 | 6.25 | 2.50 | 2.50 |
| Neusilin US2 | 15.00* | 6.00* | 6.00 |
| Sucralose | 1.25 | 0.50 | 0.50 |
| Avicel PH 101 | 12.50 | 5.00 | 5.00 |
| Magnesium stearate | 4.50 | 1.80 | 1.80 |
| Total | 250.00 | 100.00 | 100.00 |

*Neusilin quantity tripled and compensated with Pearlitol Flash

### Manufacturing Procedure:

1. Weigh the required quantities of MMT and Neusilin US2
2. Premix all the materials from step 1 in a suitable polybag and screen though 30 mesh hand screen
3. Mix the blend from step 2 in a suitable poly bag for 3-5 minutes
4. Weight the required quantities of Pearlitol Flash and screen through 20 mesh
5. Divide the Pearlitol Flash into 2 equal quantities
6. Add half of the screened Pearlitol Flash to blend from step 3, mix 3-5 minutes
7. Weigh the required quantities of menthol, sucrose and Avicel PH 101 and screen them though 20 mesh hand screen
8. Weigh the required quantities of sodium carbonate and sodium bicarbonate, screen them through 20 mesh hand screen
9. Add remaining quantity of Pearlitol Flash to step 8 materials and mix in a poly bag for 3-5 minutes
10. Add blend from steps 7, 9 to step 6, mix for 3-5 minutes
11. Weigh the required quantity of magnesium stearate and screen through 30 mesh hand screen
12. Add screened magnesium stearate from step 8 to blend from step 7 and d mix 1-2 minutes
12. Compress the blend from step 10 into 5-10 tablets on a carver press using appropriate tools to target weight of 250 mg.

### Example 11

### Granulation—Sodium Carbonate Solution (No Binder)(SBC & MMT Substrate)

| Component | mg/tablet | % w/w | g/batch |
|---|---|---|---|
| Memantine HCl | 18.12 | 7.25 | 7.25 |
| Pearlitol Flash | 172.38 | 68.95 | 68.95 |
| Sodium bicarbonate | 21.00 | 8.4 | 8.4 |
| Sodium carbonate | 9.00 | 3.6 | 3.6 |
| anhydrous | | | |
| Menthol 3433-002 | 6.25 | 2.50 | 2.50 |
| Neusilin US2 | 5.00 | 2.00 | 2.00 |
| Sucralose | 1.25 | 0.50 | 0.50 |
| Avicel PH 101 | 12.50 | 5.00 | 5.00 |
| Magnesium stearate | 4.50 | 1.80 | 1.80 |
| Total | 250.00 | 100.00 | 100.00 |

### Manufacturing Procedure:

12. Weigh the required quantities of sodium carbonate and dissolve in few ml of water
13. Weigh the required quantities of Pearlitol Flash, Avicel PH 101, sodium bicarbonate and MMT and screen them through 20 mesh hand screen separately
14. Divide Pearlitol Flash and Avicel PH 101 from step 2 into 2 equal halves
15. Add sodium bicarbonate and MMT to half of Pearlitol and Avicel and granulate with solution from step 1 in a suitable container. If necessary add additional amount of water.
16. Perform moisture analysis on the wet mass and dry in a tray dryer until the LOD less than 2-3%
17. Weigh the required quantities Neusilin US2, sucralose, and menthol and screen them through 20 mesh hand screen
18. Blend the granules from step 5, screened materials from step 6 and remaining half materials from step 53 in a suitable poly bag for 3-5 minutes
19. Weigh the required quantities of magnesium stearate and screen through 30 mesh hand screen
20. Add screened magnesium stearate from step 8 to blend from step 7 and mix 1-2 minutes
21. Compress the blend from step 9 into 5-10 tablets on a carver press using appropriate tools to target weight of 250 mg.

### Example 12

### Granulation—Sodium Carbonate Solution w/Binder (Sodium Bicarbonate & MMT Substrate)

| Component | mg/tablet | % w/w | g/batch |
|---|---|---|---|
| Memantine HCl | 18.12 | 7.25 | 7.25 |
| Pearlitol Flash | 162.38 | 64.95 | 64.95 |
| Binder | 10.00 | 4.00 | 4.00 |
| Purified water | — | — | q.s |
| Sodium bicarbonate | 21.00 | 8.4 | 8.4 |
| Sodium carbonate | 9.00 | 3.6 | 3.6 |
| anhydrous | | | |
| Menthol 3433-002 | 6.25 | 2.50 | 2.50 |
| Neusilin US2 | 5.00 | 2.00 | 2.00 |
| Sucralose | 1.25 | 0.50 | 0.50 |
| Avicel PH 101 | 12.50 | 5.00 | 5.00 |
| Magnesium stearate | 4.50 | 1.80 | 1.80 |
| Total | 250.00 | 100.00 | 100.00 |

### Manufacturing Procedure:

1. Weigh the required quantities of sodium carbonate and dissolve in few ml of water. Weigh the required amount of binder, add to the sodium carbonate solution and dissolve
2. Weigh the required quantities of Pearlitol Flash and Avicel PH 101 and screen them through 20 mesh hand screen separately
3. Divide Pearlitol Flash and Avicel PH 101 from step 2 into 2 equal halves
4. Add sodium bicarbonate and MMT to half of Pearlitol and Avicel and granulate with solution from step 1 in a suitable container. If necessary add additional amount of water.
5. Perform moisture analysis on the wet mass and dry in a tray dryer until the LOD less than 2-3%
6. Weigh the required quantities Neusilin US2, sucralose, and menthol and screen them through 20 mesh hand screen
7. Blend the granules from step 7, screened materials from step 6 and remaining half materials from step 5 in a suitable poly bag for 3-5 minutes
8. Weigh the required quantities of magnesium stearate and screen through 30 mesh hand screen
9. Add screened magnesium stearate from step 10 to blend from step 9 and mix 1-2 minutes
10. Compress the blend from step 9 into 5-10 tablets on a carver press using appropriate tools to target weight of 250 mg.

### Example 13

### Granulation—Binder Only Solution (SC/SBC Extragran)(MMT Substrate)

| Component | mg/tablet | % w/w | g/batch |
|---|---|---|---|
| Memantine HCl | 18.12 | 7.25 | 7.25 |
| Pearlitol Flash | 162.38 | 64.95 | 64.95 |
| binder | 10.00 | 4.00 | 4.00 |
| Purified water | — | — | q.s |
| Sodium bicarbonate | 21.00 | 8.4 | 8.4 |
| Sodium carbonate | 9.00 | 3.6 | 3.6 |
| anhydrous | | | |
| Menthol 3433-002 | 6.25 | 2.50 | 2.50 |
| Neusilin US2 | 5.00 | 2.00 | 2.00 |
| Sucralose | 1.25 | 0.50 | 0.50 |
| Avicel PH 101 | 12.50 | 5.00 | 5.00 |
| Magnesium stearate | 4.50 | 1.80 | 1.80 |
| Total | 250.00 | 100.00 | 100.00 |

### Manufacturing Procedure:

1. Prepare the binder solution with purified water
2. Weigh the required quantities of MMT, Pearlitol Flash and Avicel PH 101 and screen them through 20 mesh hand screen separately
3. Divide the Pearlitol Flash into 2 equal halves
4. Granulate one half of the Pearlitol Flash, Avicel PH 101 and MMT in a suitable container with binder solution from step 1. Add purified water if needed.
5. Perform moisture analysis on the wet mass and dry in a tray dryer until the LOD less than 2-3%
6. Weigh the required quantities Neusilin US2, sucralose, menthol, sodium carbonate & sodium bicarbonate and screen them through 20 mesh hand screen.
7. Blend the granules from step 5 and screened materials from step 6 and remaining half of the Pearlitol Flash from step 3 in a suitable poly bag for 3-5 minutes.
8. Weigh the required quantities of magnesium stearate and screen through 30 mesh hand screen
13. Add screened magnesium stearate from step 8 to blend from step 7 and mix 1-2 minutes
14. Compress the blend from step 9 into 5-10 tablets on a carver press using appropriate tools to target weight of 250 mg.

### Example 14

### Granulation—Binder Only Solution—(SC/SBC Substrate)(MMT Extragranular)

| Component | mg/tablet | % w/w | g/batch |
|---|---|---|---|
| Memantine HCl | 18.12 | 7.25 | 7.25 |
| (Milled) | | | |
| Pearlitol Flash | 162.38 | 64.95 | 64.95 |
| Binder | 10.00 | 4.00 | 4.00 |
| Purified water | — | — | q.s |
| Sodium bicarbonate | 21.00 | 8.4 | 8.4 |
| Sodium carbonate | 9.00 | 3.6 | 3.6 |
| anhydrous | | | |
| Menthol 3433-002 | 6.25 | 2.50 | 2.50 |
| Neusilin US2 | 5.00 | 2.00 | 2.00 |
| Sucralose | 1.25 | 0.50 | 0.50 |
| Avicel PH 101 | 12.50 | 5.00 | 5.00 |
| Magnesium stearate | 4.50 | 1.80 | 1.80 |
| Total | 250.00 | 100.00 | 100.00 |

### Manufacturing Procedure:

1. Prepare the binder solution with purified water
2. Weigh the required quantities of sodium carbonate, sodium bicarbonate, Pearlitol Flash and Avicel PH 101 and screen them through 20 mesh hand screen separately
3. Divide the Pearlitol Flash into 2 equal halves
4. Granulate one half of the Pearlitol Flash, Avicel PH 101, sodium carbonate and sodium bicarbonate in a suitable container with binder solution from step 1. Add additional purified water if needed
5. Perform moisture analysis on the wet mass and dry in a tray dryer until the LOD less than 2-3%
6. Weigh the required quantities Neusilin US2 and MMT and screen together through 30 mesh hand screen.
7. Weigh the required quantities of sucralose, menthol and screen them through 20 mesh hand screen.
8. Blend the granules from step 5 and screened materials from steps 6, 7 and remaining half of the Pearlitol Flash from step 3 in a suitable poly bag for 3-5 minutes.
9. Weigh the required quantities of magnesium stearate and screen through 30 mesh hand screen
10. Add screened magnesium stearate from step 9 to blend from step 8 and mix 1-2 minutes
11. Compress the blend from step 9 into 5-10 tablets on a carver press using appropriate tools to target weight of 250 mg.

### Example 15

### Fluid Bed Granulation MMT w/28% SC/SBC Solution (No Binder)(72% SC/SBC Extragran)

| | | |
|---|---|---|
| Ingredient Name | Qty per Unit (mg) | Quantity kg |

Granulation

| | | |
|---|---|---|
| Memantine HCl | 12.08 | 0.456* |
| Sodium bicarbonate | 1.96 | 0.148* |
| Sodium carbonate | 0.84 | 0.064* |
| anhydrous | | |
| Pearlitol Flash | 119.05 | 2.482 |
| Water purified | Evaporated | 25.704* |
| | during process | |

Blending and Lubricating

| | | |
|---|---|---|
| Pearlitol Flash | 79.37 | 1.655 |
| Sodium bicarbonate | | 0.362 |
| Sodium carbonate | | 0.156 |
| anhydrous | | |
| Cellulose | 12.50 | 0.300 |
| microcrystalline | | |
| Menthol 3433-002 | 6.25 | 0.150 |
| Neusilin US2 | 5.00 | 0.120 |
| Sucralose | 1.25 | 0.030 |
| Magnesium stearate | 4.50 | 0.108 |
| Total | 250 mg | 6.000 |

*5% excess to account for loss during the process

### Manufacturing Process

1. Add water purified to a stainless steel container. Stir the purified water using an electric mixer to form a vortex.
2. Slowly add ˜28% sodium bicarbonate into the vortex and dissolve it completely. Measure the pH.
3. Slowly add ˜28% anhydrous sodium carbonate and dissolve it completely. Measure the pH.
4. Slowly add all MMT in to Step 3 under stirring. Continue the stirring until the drug gets completely dissolved and a clear solution is obtained.
5. Pre-heat the GPCG-5 using the following process parameters
   a. Inlet Air Temperature: 50 to 90° C.
   b. Air Volume: Range 100 to 500 m³/h
   c. Atomization Pressure: 0.5 to 4.0 bar
   d. Filter Shake Interval/Duration: 3 shakes/60 sec
6. Load the Pearlitol Flash into the GPCG-5 bowl.
7. Fluidize the blend in the GPCG-5 product bowl until the product temperature has reached 35° C. Adjust exhaust air flap to maintain fluidization.
8. Start spraying the granulation solution on to the fluidized bed at the settings described below.
   a. Inlet Air Temperature: 50-90° C.
   b. Exhaust Air Flap: 5-80%
   c. Spray Rate: 10-150 g/min
   d. Air Atomization Pressure: 0.5 to 4.0 bar
9. After the entire granulating solution has been sprayed, dry the granulation in the GPCG-5 product bowl at 70° C. inlet temperature until the LOD is LT 2-3%.
10. Discharge the dried granulation and pass it through 20 mesh screen. Record the net weight after screening.
11. Calculate adjusted quantities of extra-granular excipients corresponding to the weighed quantity of the screened granules.
12. Weigh the adjusted quantities from step 12
13. Pass the following through a 20 mesh hand screen
   a. Pearlitol Flash
   b. Sodium bicarbonate
   c. Sodium carbonate anhydrous
   d. Avicel PH 101
   e. Menthol 3433-002
   f. Neusilin us2
   g. Sucralose
14. Pass the magnesium stearate 5712 through a 30 mesh screen
15. Load the following materials into the 16 qt. V-blender in the following order and blend for 12 minutes.
   a. Approximately half of the screened granulation
   b. Screened excipients from step 13
   c. Remaining half of the screened granulation
16. Add the screened magnesium stearate into the 16 qt. V-blender and blend for 1.5 minutes
17. Discharge and compress to 250 mg tablet weight and 3-4 kp hardness

### Example 16

### Fluid Bed Granulation—SC/SBC Solution w/Binder (MMT Extragran)

| | | |
|---|---|---|
| Ingredient Name | Qty per Unit (mg) | Quantity (kg) |

Granulation

| | | |
|---|---|---|
| Pearlitol Flash | 80.20 | 1.925 |
| Cellulose | 20.05 | 0.481 |
| microcrystalline | | |

Binder solution

| | | |
|---|---|---|
| Sodium bicarbonate | 7.00 | 0.176 kg⁽²⁾ |
| Sodium carbonate | 3.00 | 0.076 kg⁽²⁾ |
| anhydrous | | |
| Binder | 18.80 | 0.474 kg⁽²⁾ |
| Water purified | 896.8 ⁽¹⁾ | 22.600 kg⁽²⁾ |
| Total | 129.05 | |

⁽¹⁾ Removed by evaporation during the drying process
⁽²⁾Prepared in 5% excess

### Manufacturing Process

1. Add water purified to a suitable tared stainless steel container. Stir the purified water using an electric mixer to form a vortex.
2. Slowly add sodium bicarbonate) into the vortex and dissolve it completely. Measure the pH
3. Slowly add anhydrous sodium carbonate and dissolve it completely. Measure the pH
4. Slowly add binder into vortex of step 3 solution and continue mixing for at least 30 minutes until the solution is clear with no lumps. Measure the pH of the solution.
5. Calibrate granulating solution delivery rate using the granulating solution prior to start of granulation. Record pump RPM setting to obtain the flow rate.
6. Pre-heat the GPCG-5 using the following process parameters
   a. Inlet Air Temperature: 50 to 90° C.
   b. Air Volume: Range 100 to 500 m³/h
   c. Atomization Pressure: 0.5 to 4.0 bar
   d. Filter Shake Interval/Duration: 3 shakes/60 sec
7. Load the Pearlitol Flash into the GPCG-5 bowl.
8. Fluidize the blend in the GPCG-5 product bowl until the product temperature has reached 35° C. Adjust exhaust air flap to maintain fluidization.
9. Start spraying the granulation solution on to the fluidized bed at the settings described below.
   a. Inlet Air Temperature: 50-90° C.
   b. Exhaust Air Flap: 5-80%
   c. Spray Rate: 10-150 g/min
   d. Air Atomization Pressure: 0.5 to 4.0 bar
10. After the entire granulating solution has been sprayed, dry the granulation in the GPCG-5 product bowl at 70° C. inlet temperature until the LOD is LT 2-3%.
11. Discharge the dried granulation and pass it through 20 mesh screen. Record the net weight after screening.
12. Weight a portion of the screened granules and use it to make the Memantine tablets as follows:

| Composition | mg/tablet | % w/w | g/batch |
|---|---|---|---|
| GPCG-5 Intragranular | 129.05 | 51.62 | 51.62 |
| portion from step 12 | | | |
| Memantine HCl | 12.08 | 4.83 | 4.83 |
| Pearlitol Flash | 91.87 | 36.75 | 36.75 |
| Menthol | 6.25 | 2.50 | 2.50 |
| Neusilin US2 | 5.00 | 2.00 | 2.00 |
| Sucralose | 1.25 | 0.50 | 0.50 |
| Magnesium Stearate | 4.50 | 1.80 | 1.80 |
| Total | 250.00 mg | 100.00 | 100.00 |

13. Weigh the required quantities of GPCG-5 intragranular material and other excipients separately to make a 100 g batch
14. Premix all the excipients except magnesium stearate and pass through 20 mesh hand screen
15. Screen the intragranular portion with 20 mesh hand screen
16. Blend the materials from step 14 and 15 for 8-10 minutes using Turbula mixer
17. Screen the magnesium stearate through 30 mesh hand screen
18. Add screened magnesium stearate to step 16 and blend for 1-2 minutes using Turbula mixer
19. Compress the blend from step 18 to target weight of 250 mg and hardness of 3-4 kp.

### Example 17

### Fluid Bed Granulation—SC/SBC/MMT Solution w/Binder

| | | |
|---|---|---|
| Ingredient Name | Qty per Unit (mg) | Quantity (kg) |

Granulation

| | | |
|---|---|---|
| Pearlitol Flash | 80.20 | 1.925 |
| Cellulose | 20.05 | 0.481 |
| microcrystalline | | |

Binder solution

| | | |
|---|---|---|
| Memantine HCl | 12.08 | 0.304 |
| Sodium bicarbonate | 7.00 | 0.176⁽²⁾ |
| Sodium carbonate | 3.00 | 0.076⁽²⁾ |
| anhydrous | | |
| Binder | 18.80 | 0.474⁽²⁾ |
| Water purified | 896.8 ⁽¹⁾ | 22.600⁽²⁾ |
| Total | 141.13 | |

⁽¹⁾ Removed by evaporation during the drying process
⁽²⁾Prepared in 5% excess

### Manufacturing Process

1. Weigh the require quantities of materials as shown in the tablet above
2. Add water purified to a suitable tared stainless steel container. Stir the purified water using an electric mixer to form a vortex.
3. Slowly add sodium bicarbonate into the vortex and dissolve it completely. Measure the pH
4. Slowly add anhydrous sodium carbonate and dissolve it completely. Measure the pH
5. Slowly add Memantine HCl and dissolve it completely. Measure the pH
6. Slowly add binder into vortex of step 4 solution and continue mixing for at least 30 minutes until the solution is clear with no lumps. Measure the pH of the solution.
7. Calibrate granulating solution delivery rate using the granulating solution prior to start of granulation. Record pump RPM setting to obtain the flow rate.
8. Pre-heat the GPCG-5 using the following process parameters
   a. Inlet Air Temperature: 50 to 90° C.
   b. Air Volume: Range 100 to 500 m³/h
   c. Atomization Pressure: 0.5 to 4.0 bar
   d. Filter Shake Interval/Duration: 3 shakes/60 sec
9. Load the Pearlitol Flash into the GPCG-5 bowl.
10. Fluidize the blend in the GPCG-5 product bowl until the product temperature has reached 35° C. Adjust exhaust air flap to maintain fluidization.
11. Start spraying the granulation solution on to the fluidized bed at the settings described below.
   a. Inlet Air Temperature: 50-90° C.
   b. Exhaust Air Flap: 5-80%
   c. Spray Rate: 10-150 g/min
   d. Air Atomization Pressure: 0.5 to 4.0 bar
12. After the entire granulating solution has been sprayed, dry the granulation in the GPCG-5 product bowl at 70° C. inlet temperature until the LOD is LT 2-3%.
13. Discharge the dried granulation and pass it through 20 mesh screen. Record the net weight after screening.
14. Weight a portion of the screened granules and use it to make the memantine tablets as follows:

| Composition | mg/tablet | % w/w | g/batch |
|---|---|---|---|
| GPCG-5 Intragranular | 141.13 | 56.45 | 56.45 |
| portion from step 12 | | | |
| Pearlitol Flash | 91.87 | 36.75 | 36.75 |
| Menthol | 6.25 | 2.50 | 2.50 |
| Neusilin US2 | 5.00 | 2.00 | 2.00 |
| Sucralose | 1.25 | 0.50 | 0.50 |
| Magnesium Stearate | 4.50 | 1.80 | 1.80 |
| Total | 250.00 mg | 100.00 | 100.00 |

15. Weigh the required quantities of GPCG-5 trial intragranular material and other excipients separately to make a 100 g batch as shown in table above
16. Premix all the excipients except magnesium stearate and pass through 20 mesh hand screen
17. Screen the intragranular portion with 20 mesh hand screen
18. Blend the materials from step 14 and 15 for 8-10 minutes using Turbula mixer
19. Screen the magnesium stearate through 30 mesh hand screen
20. Add screened magnesium stearate to step 16 and blend for 1-2 minutes using Turbula mixer
21. Compress the blend from step 18 to target weight of 250 mg and hardness of 3-4 kp.

### Example 18

### Wet Granulations—MMT Gran & SC/SBC

| Trade Name | mg | 12 mg % w/w | |
|---|---|---|---|
| Memantine HCl | 12.0 | 2.4 | Memantine |
| Binder | 19.0 | 3.8 | Granulation |
| MCC | 20.0 | 4.0 | |
| Pearlitol Flash | 80.0 | 16.0 | |
| Sodium Bicarbonate | 7.0 | 1.4 | |
| Sodium Carbonate | 3.0 | 0.6 | |
| Binder | 19.0 | 3.8 | Alkalization |
| MCC | 20.0 | 4.0 | Agents |
| Pearlitol Flash | 80.0 | 16.0 | Granulation |
| Pearlitol Flash | 175.5 | 35.1 | Extragranular |
| Flavor (cherry or honey lemon) | 7.5 | 1.5 | Ingredients |
| Menthol 3433-002 | 25.0 | 8.0 | for tablet blend |
| Eucalyptus oil | 9.0 | 0.9 | |
| Neusilin US2 | 10.0 | 2.0 | |
| Sucralose | 4.0 | 0.8 | |
| Magnesium Stearate | 9.0 | 1.8 | |
| Total | 500 | 100.00 | |

### Manufacturing Process

The manufacturing process of lozenges (compressed tablets) can be described by the following unit operations

1. Memantine Granulation

2. Alkalinization Agents Granulation

3. Tablet Blending and Compression

The first unit operation consists of granulating Memantine HCl, Pearlitol Flash and microcrystalline cellulose with aqueous binder solution in a high shear mixer or Fluid bed granulator. The granules are then dried in the fluid bed at 70° C. to an LOD NMT 2-3%. After drying, the granules are milled using a Comill conical mill.
The second unit of operation consists of granulating Pearlitol Flash and microcrystalline cellulose with solution of sodium bicarbonate, sodium carbonate and binder in a high shear mixer or Fluid bed granulator. The granules are then dried in the fluid bed at 70° C. to an LOD NMT 2-3%. After drying, the granules are milled using a Comill conical mill.
The third unit of operation consists of blending the Memantine, alkalinizing agent granules, de-lumped Pearlitol Flash, Flavor, Menthol, Eucalyptus oil, Magnesium Alumino Metasilicate (Neusillin), Sucralose, in 16 quart v-blender for 10 minutes. The resulting blend is compressed to 500 mg target weight, 5-10 kp hardness.

### Example 19

### Pharmacokinetic Effect of Compressed Lozenges with Sodium Carbonate and Sodium Bicarbonate

This example reviews the pharmacokinetic effect of three different lozenge formulations with doses of 6 mg memantine: 1) compressed lozenge with sodium carbonate and sodium bicarbonate with 3 mg of sodium carbonate, 7 mg sodium bicarbonate (see Table 5); 2) compressed lozenge with sodium carbonate and sodium bicarbonate with 9 mg of sodium carbonate, 21 mg sodium bicarbonate (see Table 5); and 3) Namenda®, an oral immediate release (IR) lozenge.

This study is a controlled, randomized, open-label, parallel group study. Eligible subjects (N=5-10) will be enrolled and randomized to study treatment on Study Day 1 (randomized 1:1:1 to receive one of three lozenges described above). Subjects will have a screening visit up to twenty one (21) days prior to enrollment to ensure suitability for study participation. During the screening, subjects will be evaluated by reviewing medical history, concomitant medications, physical examination (including inspection of oral cavity), vital signs (blood pressure, temperature and pulse only), height and weight, 12-lead electrocardiogram (ECG), standard laboratory assessments and urinalysis, and drug and alcohol screens. Subjects will arrive at the clinical research unit (CRU) in the fasted state at least 2 hours prior to dosing on Day 1, and will remain in the CRU under supervision for up to 12 hours. On Day 1, overnight fasted subjects will be randomized in a 1:1:1 manner to one of the three lozenge formulations, administered sublingually or buccally. Pharmacokinetic blood samples to assess memantine plasma concentrations will be collected at pre-determined time-points over 8-72 hours post-dose. Subjects will remain in the CRU until the 8 hour blood sample has been obtained (Day 1). A follow-up visit will be made on Day 2, 24±1 hr hours post dosing. Clinical exams and safety assessments (including inspection of oral cavity) will be conducted twice on Day 1 and at the return visit on Day 2 (or Day 3 for the 72 hr sample). A pharmacokinetic blood samples will be collected 24±1 hr hours post dosing on Day 2. Subjects will be discharged from the CRU after the 8 hour sample on Day 1 and from the study on Day 2.

Plasma samples will be assayed for memantine using validated liquid chromatography with tandem mass spectrometry (LC-MS/MS) validated methods. The plasma concentration-time data following administration of memantine will be analyzed. Actual sampling times will be used for all individual listings and plots of plasma concentration data. The blood samples are used to test the following Cmax, Tmax; and AUC pharmacokinetic parameters for each formulation. The results are indicated in Table 6 below. The results demonstrate that both compressed lozenges with sodium carbonate and sodium bicarbonate, when administered sublingually or buccally, provide a substantially shorter Tmax than Namenda® IR. The test also demonstrates that both compressed lozenges with sodium carbonate and sodium bicarbonate provide a higher AUC_{0-1 hr} and AUC_{0-2 hr} than Namenda® IR. This indicates that the compressed lozenges provide a higher rate of memantine absorption relative to Namenda® IR.

TABLE 6
Mean Pharmacokinetic Data of Compressed Lozenges with Sodium Carbonate and
Sodium Bicarbonate vs. Namenda ® IR (at comparable strengths)

| | Cₘₐₓ | Tₘₐₓ | AUC₀₋₁ | AUC₀₋₂ | Ka | Tᵢₙᵢₜᵢₐₗ | Cᵢₙᵢₜᵢₐₗ |
|---|---|---|---|---|---|---|---|
| | (ng/mL) | (hr) | (ng · hr/mL) | (ng · hr/mL) | (h⁻¹) | (min) | (ng/mL) |
| Lozenge with 9 mg of | 9.0 | 3.8 | 4.0 | 11 | 6.8 | 5 | 2.5 |
| sodium carbonate, 21 mg | | | | | | | |
| sodium bicarbonate/ | | | | | | | |
| Sublingual administration | | | | | | | |
| Lozenge with 9 mg of | 8.3 | 5.4 | 2.8 | 7.1 | 0.85 | 30 | 3.99 |
| sodium carbonate, 21 mg | | | | | | | |
| sodium bicarbonate/ | | | | | | | |
| Buccal administration | | | | | | | |
| Lozenge with 3 mg of | 10.5 | 4.8 | 1.8 | 6.5 | 0.43 | 10 | 1.64 |
| sodium carbonate, 7 mg | | | | | | | |
| sodium bicarbonate/ | | | | | | | |
| Sublingual administration | | | | | | | |
| Tablet, Namenda ® IR | 7.8 | 5.6 | 1.1 | 5.6 | 0.61 | 180 | 2.25 |

Tᵢₙᵢₜᵢₐₗ, first timepoint where all subjects within a treatment group demonstrated a quantifiable concentration;
Cᵢₙᵢₜᵢₐₗ, Initial concentration at that point.
The plasma levels of memantine are predicted using a single compartment first order input and output kinetic model of the data for a 6 mg memantine lozenge using the following equation:
Ci = (D × K_{A}/V)/(K_{A} − K_{E}) × {EXP (−K_{E} × t) − EXP (−K_{A} × t)} Wherein Ci is predicted memantine plasma concentration, D is dose, V is apparent Volume of distribution, t is time, K_{A} is absorption rate constant, and K_{E} is the elimination rate constant.

### Example 20

### Efficacy Investigation of Compressed Lozenge

This example provides an evaluation of the dose-dependent antitussive effects and safety/tolerability of a compressed lozenge formulation with 3 mg of sodium carbonate and 7 mg sodium bicarbonate (see Table 5). The dose-dependent study reviews doses of 6.0 mg memantine and 12.0 mg memantine in the formulation in comparison with a placebo with no memantine in subjects with cough due to upper respiratory tract infection.

### Methods

This study is a randomized, placebo controlled, double-blind, multicenter study in subjects with cough associated with upper respiratory tract infection. The study objectives are to determine the antitussive effect and dose response of 6 mg and 12 mg memantine dosage amounts in subjects with cough when compared to placebo and to demonstrate the safety and tolerability of 6 mg and 12 mg memantine dose lozenge in these subjects. Subjects (N=192) will have a screening visit up to two days prior to enrollment to ensure suitability for study participation. During the screening, subjects will be evaluated with standard clinical and laboratory testing, receive a chest X-ray, be asked to complete a Cough Severity Visual Analogue Scale and a Leicester Cough Questionnaire-acute (LCQ-acute) and estimate, on average and in recent memory, for how many days they tend to cough when afflicted by the common cold. On Day 1, subjects will be admitted to the Clinical Unit and randomized in a 1:1:1 manner for one of three treatment regimens for Day 2 as follows: 1) a compressed lozenge with 6 mg memantine; 2) a compressed lozenge with 12 mg memantine; or 3) a placebo lozenge with no memantine. Physical exams and safety assessments will be conducted. Cough recordings will be made using a cough monitor which records from a sensor that measures biological sounds. Immediately after initiating cough monitoring, dosing will be initiated in a double-blind manner according to the randomization assignment (compressed lozenge 6 mg, compressed lozenge 12 mg, or placebo). Blood samples will be collected 1 hour after the 6th study dose (third dose on study Day 2) for determination of memantine plasma concentrations. The subjects will be confined to the clinic for a 48-hour period (beginning on Day 1, when they are admitted to the Clinical Unit) during which automated cough counts and serial visual analogue scales will be collected. Vital signs and buccal inspections will be performed at screening, on both treatment days (Day 1 and Day 2) and at time of study discharge (Day 3).

The cough monitoring device will be used to determine the cough counts. Sound recording equipment will record digital audio files which will be transferred securely to the Central Laboratory, where they will be processed and analyzed to determine individual cough counts throughout the 48-hour recording period. In a series of individual coughs, each expiratory event associated with a characteristic explosive cough sound will be counted as one cough.

The efficacy analysis indicates that the change in hourly cough frequency in subjects is sufficiently reduced after 24 hrs for subjects taking the 6 mg memantine compressed lozenge over the placebo. The efficacy analysis also indicates that the hourly cough frequency in subjects is even more reduced after 24 hrs for subjects taking the 12 mg memantine compressed lozenge relative to the 6 mg memantine compressed lozenge. Regarding safety, there was no indication of the 12 mg memantine compressed lozenge or the 6 mg memantine compressed lozenge creating adverse side effects relative to the placebo.

### Example 21

### Efficacy Investigation of Compressed Lozenge in Chronic Cough

This example provides an evaluation of the dose-dependent antitussive effects and safety/tolerability of the 6.0 mg and 12.0 mg compressed lozenge formulation with 9 mg of sodium carbonate and 21 mg sodium bicarbonate (see Table 5) in subjects with chronic cough.

### Methods

This study is a randomized, placebo-controlled, double-blind, crossover study of compressed lozenges in subjects with chronic refractory cough. The study objectives were to determine the antitussive effect size and dose response of compressed lozenges in subjects with chronic cough and to demonstrate the safety and tolerability of compressed lozenges in subjects with chronic cough. Approximately seventy (70) subjects will be enrolled in this multi-center, randomized, crossover, double-blind, placebo-controlled study to complete at least 50 subjects. Subjects will be randomized to receive lozenges with 6 mg memantine, 12 mg memantine, or a matching placebo for 2 weeks (first treatment period), and after a 2 week washout (i.e., no administration of the drug), subjects will be crossed over to receive matching placebo or lozenges 6 mg or 12 mg for another 2 weeks (second treatment period). On the first 2 days of each treatment period, study medication will be administered once a day followed by 2 doses a day for the next 3 days, and then 3 doses per day until the last dosing day of the treatment period when 2 doses will be administered and a clinic visit will be completed. Automated cough counting, Visual Analogue Scale (VAS), Cough Severity Diary (CSD), and Leicester Cough Questionnaire (LCQ) will be performed at the beginning of the study and upon conclusion of the first treatment period, washout, and the second treatment period. Blood will be drawn for blood concentrations of study drug on the last day of each treatment period.

Diagnosis and main criteria for inclusion in this trial will include chronic refractory cough of >8 weeks duration where underlying etiology has been treated and yet cough persists, i.e. cough must not be the result of inadequate treatment of the underlying etiology. Underlying etiologies can include gastroesophogeal reflux (GERD), post nasal drip syndrome (PNDS), persistent post-infectious cough, asthma, nonasthmatic eosinophilic bronchitis, etc., diagnosed by clinical criteria. Subjects with idiopathic chronic cough are eligible for the study. Subjects must have a cough severity threshold (VAS) greater than 35 mm and a mean CSD frequency domain score greater than 3.0 during screening.

The cough monitoring device will be used to determine the cough counts. Sound recording equipment will record digital audio files which will be transferred securely to the Central Laboratory, where they will be processed and analyzed to determine individual cough counts throughout the 24-hour recording period. In a series of individual coughs, each expiratory event associated with a characteristic explosive cough sound will be counted as one cough.

The study indicates that cough frequency in subjects in various periods (e.g. awake, sleep, and total 24 hr periods) is significantly reduced after the treatment period for subjects taking the 6 mg memantine compressed lozenge relative to the placebo. The study indicates that cough frequency in subjects is reduced even further after the treatment period for subjects taking the 12 mg memantine compressed lozenge relative to the 6 mg compressed lozenge. Regarding safety, there was no indication of the 12 mg memantine compressed lozenge or the 6 mg memantine compressed lozenge creating adverse side effects relative to the placebo at any time point in the study.

### Example 22

### Manufacturing Process of Memantine HCl Solution Sodium Bicarbonate/Sodium Carbonate 9/1 Buffer Solution

A memantine solution may be prepared as described below. First, make a sodium bicarbonate/sodium carbonate (SB/SC) 9/1 buffer solution as follows:
1. Into a 1000 mL beaker, add 495.7 g of sterile water.
2. Add 3.78 g of sodium bicarbonate and 0.53 g sodium carbonate to the vortex. Mix until the solution becomes clear (approximately 5 minutes).
3. Solution may be set aside at room temperature until required for preparation of the memantine solution described below.
   Second, prepare a mixture of Memantine HCl (12 mg/mL) in 0.1 M sodium bicarbonate/carbonate buffer as follows:
1. Into a 400 mL beaker, add approximately 170-180 g of the SB/SC 9/1 buffer solution described above.
2. Bring the water to a vigorous vortex. Maintain the vortex for the duration of preparation.
3. Add 2.40 g of Memantine HCl to the vortex. Mix until the solution becomes clear (approximately 30-60 minutes).
4. QS the solution to 200 g net weight with SB/SC 9/1 buffer. Mix for an additional 5 minutes. Record the pH. The pH should range from about 8-10.
5. Transfer the remaining solution into aliquots necessary for execution of the clinical study and/or bulk container (60 cc glass container). Aliquot the memantine solution for a total 6 mg memantine pH (8-10) per dose. Label the aliquots and containers appropriately and store in a secure area at ambient room temperature ˜25° C.

### Example 23

### Pharmacokinetic Effect of Liquid Formulation of Memantine with Sodium Carbonate and Sodium Bicarbonate

This example reviews the pharmacokinetic effect of liquid formulation of 6 mg memantine with pH of 8.0-10.0 as provided in Example 22.

This study was a controlled, open-label study. Eligible subjects (N=5) were enrolled to study treatment on Study Day 1. Subjects had a screening visit up to twenty one (21) days prior to enrollment to ensure suitability for study participation. During the screening, subjects were evaluated by reviewing medical history, concomitant medications, physical examination (including inspection of oral cavity), vital signs (blood pressure, temperature and pulse only), height and weight, 12-lead electrocardiogram (ECG), standard laboratory assessments and urinalysis, and drug and alcohol screens. Subjects arrived at the clinical research unit (CRU) in the fasted state at least 2 hours prior to dosing on Day 1, and remained in the CRU under supervision for up to 12 hours. Pharmacokinetic blood samples to assess memantine plasma concentrations were collected at pre-determined time-points over 8-24 hours post-dose. Subjects remained in the CRU until the 8 hour blood sample has been obtained (Day 1). A follow-up visit was made on Day 2, 24±1 hr hours post dosing. Clinical exams and safety assessments (including inspection of oral cavity) were conducted twice on Day 1 and at the return visit on Day 2 (or Day 3 for the 72 hr sample). Pharmacokinetic blood samples were collected 24±1 hr hours post dosing on Day 2. Subjects were discharged from the CRU after the 8 hour sample on Day 1 and from the study on Day 2.

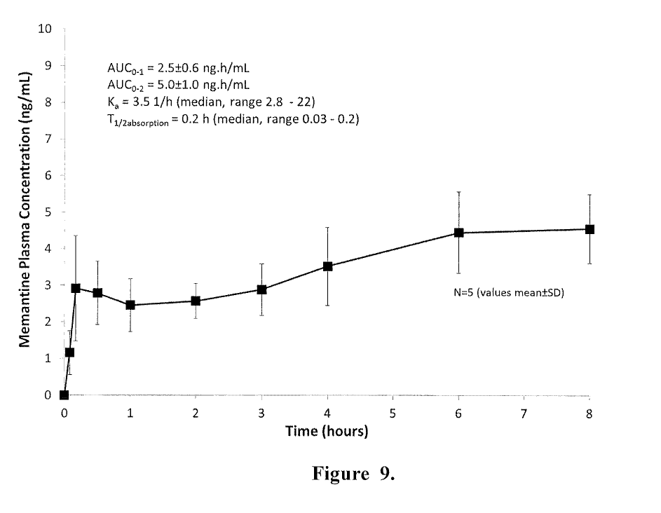

Plasma samples were assayed for memantine using validated liquid chromatography with tandem mass spectrometry (LC-MS/MS) validated methods. The plasma concentration-time data following administration of memantine were analyzed. Actual sampling times were used for all individual listings and plots of plasma concentration data. The blood samples are used to test the following Cmax, Tmax, and AUC₀₋₁, and AUC₀₋₂, and Kₐ pharmacokinetic parameters. The plasma concentration results are indicated in FIG. 9. The results demonstrate that oral solution of memantine with sodium carbonate and sodium bicarbonate, when administered sublingually, accelerates absorption of memantine and enhances early plasma exposures. The test also demonstrates that liquid formulation with sodium carbonate and sodium bicarbonate provides a marked increase in AUC_{0-1 hr} and AUC_{0-2 hr}, and absorptions rates (FIG. 9 inset text).

The plasma levels of memantine are predicted using a single compartment first order input and output kinetic model of the data for a 6 mg memantine solution using the following equation:

Ci=(D×K_{A}/V)/(K_{A}−K_{E})×{EXP(−K_{E}×t)−EXP(−K_{A}×t)}
Wherein Ci is predicted memantine plasma concentration, D is dose, V is apparent Volume of distribution, t is time, K_{A} is absorption rate constant, and K_{E} is the elimination rate constant.

We claim:

1. A compressed antitussive lozenge comprising:
memantine, or a pharmaceutically acceptable salt thereof; menthol; and an alkalinizing agent, wherein after a single buccal or sublingual administration, said compressed antitussive lozenge provides a memantine AUC_{0-1 hr} ranging from about 1.0 ng-hr/mL to about 10 ng-hr/mL.

2. The compressed antitussive lozenge of claim 1, wherein after a single buccal or sublingual administration to a patient, said compressed antitussive lozenge provides a memantine AUC_{0-2 hr} ranging from about 5.0 ng-hr/mL to about 15 ng-hr/mL.

3. The compressed antitussive lozenge of claim 1, wherein after a single buccal or sublingual administration to a patient, said compressed antitussive lozenge provides a memantine AUC_{0-3 hr} ranging from about 12.0 ng-hr/mL to about 20 ng-hr/mL.

4. The compressed antitussive lozenge of claim 1, wherein said alkalinizing agent is selected from one or more from the group consisting of aluminum carbonate, aluminum hydroxide, ammonium carbonate, ammonium solution, calcium carbonate, calcium phosphate, diethanolamine, magnesium carbonate, magnesium hydroxide, magnesium oxide, magnesium trisilicate, monoethanolamine, potassium bicarbonate, potassium carbonate, potassium citrate, potassium hydroxide, sodium acetate, sodium bicarbonate, sodium carbonate, sodium citrate, sodium hydroxide, sodium phosphate dibasic, sodium phosphate monobasic, sodium phosphate tribasic, triethanolamine, tromethane and buffering agents sodium carbonate/sodium bicarbonate, barbitone sodium/hydrochloric acid, trisaminomethane/hydrochloric acid, sodium tetraborate/hydrochloric acid, glycine/sodium hydroxide, sodium carbonate/sodium hydrogen carbonate, sodium tetraborate/sodium hydroxide, sodium bicarbonate/sodium hydroxide, sodium hydrogen orthophosphate/sodium hydroxide, and potassium chloride/sodium hydroxide.

5. The compressed antitussive lozenge of claim 4, wherein said alkalinizing agent is sodium carbonate and sodium bicarbonate.

6. The compressed antitussive lozenge of claim 5, wherein the total weight of the said compressed antitussive lozenge is about 0.1 g to about 0.5.

7. The compressed antitussive lozenge of claim 5, wherein the total weight of said sodium carbonate and sodium bicarbonate in said lozenge is about 1 mg to about 40 mg.

8. The compressed antitussive lozenge of claim 7, wherein said sodium carbonate is present in an amount of about 1 mg to about 12 mg and said sodium bicarbonate is present in an amount of about 5 mg to about 25 mg.

9. The compressed antitussive lozenge of claim 8, wherein said sodium carbonate is present in an amount of about 2 mg to about 4 mg and said sodium bicarbonate is present in an amount of about 5 mg to about 10 mg.

10. The compressed antitussive lozenge of claim 8, wherein said sodium carbonate is present in an amount of about 7 mg to about 11 mg and said sodium bicarbonate is present in an amount of about 18 mg to about 24 mg.

11. The compressed antitussive lozenge of claim 1, wherein the amount of memantine is about 1 mg to about 40 mg.

12. The compressed antitussive lozenge of claim 1, wherein the amount of memantine is about 6 mg to about 9 mg.

13. The compressed antitussive lozenge of claim 1, wherein after a single buccal or sublingual administration to a patient, said compressed antitussive lozenge provides a memantine Tₘₐₓ ranging from about 10 minutes to about 5.5 hours.

14. The compressed antitussive lozenge of claim 13, wherein after a single buccal or sublingual administration to a patient, said compressed antitussive lozenge provides a memantine Tₘₐₓ ranging from about 10 minutes to about 1.5 hours.

15. The compressed antitussive lozenge of claim 13, wherein after a single buccal or sublingual administration to a patient, said compressed antitussive lozenge provides a memantine Tₘₐₓ ranging from about 2 hours to about 5.5 hours.

16. The compressed antitussive lozenge of claim 1, wherein after a single buccal or sublingual administration to a patient, said compressed antitussive lozenge provides a memantine Cₘₐₓ ranging from about 1 ng/mL to about 2.5 ng/mL per mg dosed.

17. The compressed antitussive lozenge of claim 1, wherein after a single buccal or sublingual administration to a patient, said compressed antitussive lozenge provides a memantine AUC_{0-∞} ranging from about 300 ng-hr/mL to about 1,500 ng-hr/mL.

18. The compressed antitussive lozenge of claim 1, wherein after a single buccal or sublingual administration to a patient, said compressed antitussive lozenge provides a time/plasma concentration curve with two or more peaks.

19. The compressed antitussive lozenge of claim 18, wherein after a single buccal or sublingual administration to a patient, said lozenge provides a memantine T₁ ranging from about 10 minutes to about 1.5 hours.

20. The compressed antitussive lozenge of claim 18, wherein after a single buccal or sublingual administration to a patient, said compressed antitussive lozenge provides a memantine T₂ ranging from about 2 hours to about 5.5 hours.

21. The compressed antitussive lozenge of claim 18, wherein after a single buccal or sublingual administration to a patient, said compressed antitussive lozenge provides a memantine T₁ ranging from about 10 minutes to about 1.5 hours and a memantine T₂ ranging from about 2 hours to about 5.5 hours.

22. The compressed antitussive lozenge of claim 1, wherein said compressed antitussive lozenge dissolves within about 15 minutes.

23. The compressed antitussive lozenge of claim 1, wherein said compressed antitussive lozenge further comprises one or more excipients selected from the group consisting of a binder, a sugar or sugar substitutes, a filler, a disintegrant, a lubricant, a moisture scavenger and combinations thereof.

24. The compressed antitussive lozenge of claim 23, wherein said excipients comprise microcrystalline cellulose, magnesium stearate, starch, mannitol, sucralose, and magnesium aluminometasilicate.

25. The compressed antitussive lozenge of claim 1, further comprising one or more additional pharmaceutically active ingredients selected from the group consisting of antitussives other than memantine, expectorants, mucolytics, decongestants, nasal decongestants, first generation antihistamines, antihistamines, opioid analgesics, non-opiate analgesics, antipyretics, and combinations thereof.

26. The compressed antitussive lozenge of claim 25, wherein the one or more additional pharmaceutically active ingredients are selected from the group consisting of guaifenesin, ambroxol, a first generation antihistamine, and combinations thereof.

27. A method of treating cough, comprising administering the compressed antitussive lozenge of claim 1 to the oral cavity of a patient in need thereof.

28. The method of treating cough of claim 27, wherein the oral administration is buccal administration.

29. The method of treating cough of claim 27, wherein the oral administration is sublingual administration.

30. The method of treating cough of claim 27, wherein after a single buccal or sublingual administration to a patient, said compressed antitussive lozenge provides a memantine Tₘₐₓ ranging from about 10 minutes to about 5.5 hours.

31. The method of treating cough of claim 30, wherein after a single buccal or sublingual administration to a patient, said compressed antitussive lozenge provides a memantine Tₘₐₓ ranging from about 10 minutes to about 1.5 hours.

32. The method of treating cough of claim 30, wherein after a single buccal or sublingual administration to a patient, said compressed antitussive lozenge provides a memantine Tₘₐₓ ranging from about 2 hours to about 5.5 hours.

33. The method of treating cough of claim 27, wherein after a single buccal or sublingual administration to a patient, said compressed antitussive lozenge provides a memantine Cₘₐₓ ranging from about 1 ng/mL to about 2.5 ng/mL per mg dosed and a memantine AUC_{0-∞} ranging from about 300 ng-hr/mL to about 1,500 ng-hr/mL.

34. The method of treating cough of claim 27, wherein after a single buccal or sublingual administration to a patient, said compressed antitussive lozenge provides a time/plasma concentration curve with two or more peaks.

35. The method of treating cough of claim 34, wherein after a single buccal or sublingual administration to a patient, said compressed antitussive lozenge provides a memantine T₁ ranging from about 10 minutes to about 1.5 hours.

36. The method of treating cough of claim 35, wherein after a single buccal or sublingual administration to a patient, said compressed antitussive lozenge provides a memantine T₂ ranging from about 2 hours to about 5.5 hours.

37. The method of treating cough of claim 35, wherein after a single buccal or sublingual administration to a patient, said compressed antitussive lozenge provides a memantine T₁ ranging from about 10 minutes to about 1.5 hours and a memantine T₂ ranging from about 2 hours to about 5.5 hours.

38. The method of treating cough of claim 34, wherein after a single buccal or sublingual administration to a patient, said compressed antitussive lozenge provides a memantine AUC_{0-2 hr} ranging from about 5.0 ng-hr/mL to about 15 ng-hr/mL.

39. The method of treating cough of claim 27, wherein said compressed antitussive lozenge is administered once a day.

40. The method of treating cough of claim 27, wherein said compressed antitussive lozenge is administered at least twice a day.
